(19) United States
(12) Patent Application Publication
(10) Pub. No.: US 2023/0072164 A1
(43) Pub. Date: Mar. 9, 2023

(54) GRANULAR PHARMACEUTICAL PRODUCT FOR ORAL ADMINISTRATION FROM A PRE-FILLED STRAW AND METHOD OF MANUFACTURING SUCH PHARMACEUTICAL PRODUCT

(71) Applicant: ALTERNO LABS D.O.O., Crnuce, Ljubljana (SI)

(72) Inventors: Odon PLANINSEK, Ljubljana (SI); Tilen SIMSIC, Ljubljana (SI); Boris NOLIMAL, Ljubljana (SI)

(73) Assignee: ALTERNO LABS D.O.O., Crnuce, Ljubljana (SI)

(21) Appl. No.: 17/759,688

(22) PCT Filed: Jan. 28, 2021
(86) PCT No.: PCT/EP2021/051978

§ 371 Date: Jul. 28, 2022

(30) Foreign Application Priority Data
Jan. 29, 2020 (GB) 2001237.3

(51) Int. Cl. A61K 9/50; A61K 9/00; A61J 7/00; A23P 20/10; A23P 10/10

(52) U.S. Cl. CPC A61K 9/5073; A61K 9/0053; A61K 9/5015; A61K 9/5026; A61K 9/0095; A61J 7/0038; A23P 20/10; A23P 10/10

(57) ABSTRACT

The present invention relates to a pharmaceutical formulation suitable for use in a straw suitable for oral administration of said pharmaceutical formulation, wherein the pharmaceutical formulation is a granular solid comprising a core, an optional first coating layer, and a second coating layer. The present invention also relates to a straw suitable for oral administration of such a pharmaceutical formulation, the use of said pharmaceutical formulation in the treatment of a condition in a subject in need thereof, and a method for preparing said pharmaceutical composition.

### FIELD OF THE INVENTION

The present invention relates to a pharmaceutical formulation suitable for use in a straw suitable for oral administration of said pharmaceutical formulation, wherein the pharmaceutical formulation is a granular solid comprising a core, an optional first coating layer, and a second coating layer. The present invention also relates to a straw suitable for oral administration of such a pharmaceutical formulation, the use of said pharmaceutical formulation in the treatment of a condition in a subject in need thereof, and a method for preparing said pharmaceutical composition.

### BACKGROUND TO THE INVENTION

Pre-filled straws are described in the art. For example, in US 2003/0071136 A1 a straw is described with one valve closure impressed into the body of the straw. In

CA 2230851 a drink container is described with mouthpiece with inserted valve. Valves and/or filters are added to the straw, meaning that straws and valves and optionally filters are produced separately and the straw is assembled from separate parts in later. Such straw designs use one-way valves on either inlet or outlet, and use different types of closures, i.e. caps, grids and/or filters of different mesh sizes as closures of other opening. Generally, the straw is assembled from the straw body and the closure mechanism which is inserted into the straw—either valve or filter or other form of barrier. Pre-filled straws are also described in WO 2017/111704.

In the pharmaceutical industry, a high shear apparatus can be used for wet granulation, melt granulation or melt coating. During wet granulation, granules are formed either by layering or coalescence (see B. J. Ennis, Theory of Granulation: An Engineering Perspective, in Handbook of Pharmaceutical Granulation Theory, D. M. Parikh, Editor. 2005, Taylor & Francis Group, LLC: Boca Raton.)

Granulation is the process of building up an optimum-sized, nearly spherical product from fines, melts or slurries. Granulation is brought about when a bed of solid particles moves, with simultaneous intensive mixing, in the presence of a liquid phase. This motion provides particle collisions and individual particles coalesce and bind together. Further granule growth takes place by layering on to these nuclei (see Handbook of Powder Technology, Edited by A. D. Salman, M. J. Hounslow, J. P. K. Seville; Volume 11, Page 220 (2007)).

Layering is the formation of a fresh layer of powder around an existing granule, causing slow growth. Coalescence, on the other hand, involves the collision and sticking together of two granules, causing granules to grow rapidly. In a high-shear mixer, both coalescence and layering occur at the same time scale due to strong agitation by the impeler.

The present inventors have surprisingly discovered that when particles of an active pharmaceutical ingredient (API), food supplement or vitamin that are large enough to act as nuclei are mixed with smaller sugar or sugar alcohol particles in a high shear granulator, or a high shear mixer using water or aqueous binder solution as a granulation liquid, granulation is not observed. Rather, the large API, food supplement or vitamin particles are coated with the smaller sugar and/or sugar alcohol particles. The API, food supplement or vitamin particles may be granules, crystals or pellets, and may be coated with a polymer layer. Thus, a new method has been developed for wet coating of these granules, crystals or pellets using a high shear granulator. This method enabled the formation of particles which had surprisingly beneficial properties when used in a drinking straw. In particular, a lower vacuum pressure is required to ingest a formulation comprising these particles from a straw, and a smaller volume of liquid is required to pass through the straw to administer the formulation. The formulation is considered to be “in use in the straw” when a pressure gradient is applied along the length of the straw containing the formulation, e.g. by a person sucking on one end of the straw when the opposing end is inserted into a liquid.

The coating on particles obtained in high shear mixer can be detected as a rough surface with detectable small primary particles that are adhered onto large core particles in one or more layers using optical or electron microscopy.

### SUMMARY OF THE INVENTION

In one aspect, the present invention provides a pharmaceutical formulation suitable for use in a straw suitable for oral administration of said pharmaceutical formulation, wherein the pharmaceutical formulation is solid and comprises granules, and the granules comprise:
(a) a core comprising an active pharmaceutical ingredient (API), food supplement or vitamin;
(b) optionally, a first coating layer surrounding the core; and
(c) a second coating layer surrounding the first coating layer and/or the core, the second coating layer comprising particles of a sugar, a sugar alcohol, or any mixture thereof.

In a further aspect, the present invention provides a device suitable for oral administration of a pharmaceutical formulation, wherein the device contains the pharmaceutical formulation of the invention. Preferably, the device is a straw.

In another aspect, the present invention provides a pharmaceutical formulation according to the invention for use in the treatment of a condition in a subject in need thereof, wherein the pharmaceutical formulation comprises an API, and said treatment comprises oral administration of the pharmaceutical formulation using a straw as defined herein, wherein the straw contains the pharmaceutical formulation.

In another aspect, the present invention provides a method of treating a condition in a subject in need thereof, said method comprising oral administration of a pharmaceutical formulation according to the invention using a straw as defined herein, wherein the pharmaceutical formulation comprises an API, and the straw contains the pharmaceutical formulation.

In another aspect, the present invention provides the use of a pharmaceutical formulation according to the invention for the manufacture of a medicament for the treatment of a condition in a subject in need thereof, wherein the pharmaceutical formulation comprises an API, and said treatment comprises oral administration of the pharmaceutical formulation using a straw as defined herein, wherein the straw contains the pharmaceutical formulation.

In yet another aspect, the present invention provides a method of preparing a pharmaceutical composition of the invention, said method comprising:
(a) providing granules, crystals or pellets of an active pharmaceutical ingredient (API), food supplement or vitamin;
(b) optionally, applying a first coating layer to the said granules, crystals or pellets, optionally using fluid bed coating or high shear melt coating; and
(c) applying a second coating layer using a wet coating method in a high shear mixer, wherein the second coating layer comprises particles of a sugar, sugar alcohol, or any mixture thereof.

### BRIEF DESCRIPTION OF THE DRAWINGS

FIG. 1 shows an example of the straw body.

FIG. 2 shows examples of couplings between segments of a straw body.

FIG. 3 shows examples of cross slit valves that may be used.

FIG. 4 shows a perspective view of an example of an inlet valve.

FIG. 5 shows the inlet valve of FIG. 4. in cross-section.

FIG. 6 shows an example of an outlet valve in cross-section.

FIG. 7 shows injection moulding of a straw body.

FIG. 8 shows injection moulding of a valve.

FIG. 9 shows an experimental set-up for measuring pressure in a straw.

FIG. 10 shows the general behaviour of pressure change during an experiment in which water flows through a drinking straw containing a formulation according to the invention. p₁=change in absolute pressure just before the top valve; p₂=maximum change of absolute pressure during the experiment; p₃=change of absolute pressure when the straw is empty; V=flush volume.

FIG. 11 shows a scanning electron microscope (SEM) image of paracetamol crystals, SEM images of the surface of polymer-coated paracetamol crystals, and SEM images of the cross-section of polymer-coated paracetamol crystals, all at magnification ×150.

FIG. 12 shows SEM images of trehalose and erythritol wet-coated granules having a rough surface containing trehalose erythritol primary particles at magnification ×150.

FIG. 13 shows SEM images at magnification ×150 of granules of wet coated particles with observed rough surface containing erythritol primary particles, formed in a wet coating process wherein (a) a small amount of liquid binding solution comprising maltitol is employed, and (b) a larger amount of liquid bind solution comprising maltitol is employed.

FIG. 14 shows optical microscope images at magnification ×32 of (a) paracetamol crystals, (b) polymer-coated paracetamol crystals, and (c) paracetamol granules with a first polymer coating layer and a second layer comprising erythritol and maltitol.

FIG. 15 shows the rate of release of paracetamol from polymer-coated paracetamol crystals in water, with uncoated paracetamol crystals as a reference.

FIG. 16 shows the rate of release of paracetamol from polymer-coated paracetamol crystals in a hydrochloric acid solution at pH 1.2, with Calpol solution as a reference.

FIG. 17 shows the rate of release of paracetamol from polymer-coated paracetamol crystals in a phosphate buffer solution at pH 4.5, with Calpol solution as a reference.

FIG. 18 shows (a) a conventional granulation-coating sequence, (b) the innovative coating-granulation process scheme, using the example of an API-coated pellet as the core substance.

FIG. 19 shows optical microscope images of coated paracetamol crystals (left) and 20% sucrose granulates of coated paracetamol crystals (right).

FIG. 20 shows an experimental set-up for the water sorption test.

FIG. 21 shows an example of a granulate sample with ideal straw behaviour, where k₁ is the water absorption rate and k₂ is the particle dissolving rate during water sorption measurement.

FIG. 22 shows water sorption curves for two types of coated particles and the corresponding sucrose granulates containing 20% of the coated particles by weight: (a) Kollicoat® Protect-coated paracetamol crystals (20% paracetamol by weight) in a sucrose granulate; (b) non-granulated Kollicoat® Protect-coated paracetamol crystals; (c) Actimask-coated paracetamol crystals (20% paracetamol by weight) in a sucrose granulate; and (d) non-granulated Actimask-coated paracetamol crystals.

FIG. 23 shows water sorption curves for Tachipirina and Aspirin Direct, compared with a gelatin-coated paracetamol granulate (20% API by weight) of the present invention.

FIG. 24 shows water sorption curves for three different granulate samples with differing straw behaviour: (a) sucrose granulate with 20% content of gelatin-coated particles; (b) sucrose granulate with 40% ibuprofen content; and (c) sucrose granulate with 10% stearic acid content.

FIG. 25 shows pressure measurements p₁, p₂ and p₃ for individual samples when water is sucked through a straw containing different example formulations. Each formulation is tested at two different particle sizes, 250-500 μm and 1120-1400 μm.

FIG. 26 is a bar graph illustrating values of V and p1 for a variety of different example formulations having a particle size of 250-500 μm.

FIG. 27 is a bar graph illustrating values of V and p1 for a variety of different example formulations having a particle size of 1120-1400 μm.

FIG. 28 is a line graph showing the impact on maximal siping pressure for different particle size distributions caused by (a) volume of straw filling, (b) the addition of PVP K30 as a binder, (c) the addition of cefprozil monohydrate as API and (d) the addition of SDS as a surfactant.

FIG. 29 is a line graph showing the impact on flush volume for different particle size distributions caused by (a) volume of straw filling, (b) the addition of PVP K30 as a binder, (c) the addition of cefprozil monohydrate as API and (d) the addition of SDS as a surfactant.

### DETAILED DESCRIPTION OF THE INVENTION

### Definitions

As defined herein, the term “alkyl” refers to a linear or branched saturated monovalent hydrocarbon radical. Typically, the term “alkyl” refers to a linear or branched saturated monovalent hydrocarbon radical having from 1 to 20 carbon atoms, unless otherwise specified. Examples of alkyl groups include, but are not limited to, methyl, ethyl, n-propyl, iso-propyl, n-butyl, iso-butyl, tent-butyl, and the like.

As used herein, term “active pharmaceutical ingredient” refers to any agent other than a foodstuff or vitamin which promotes a structural and/or functional change in and/or on bodies to which it has been administered.

As defined herein, the term “acyl” refers to a —COR radical, wherein R is alkyl, haloalkyl, cycloalkyl, cycloalkylalkyl, aryl, aralkyl, heteroaryl, heteroaralkyl, heterocyclyl, or heterocyclylalkyl, each as defined herein, or poly(ethylene glycol), and wherein R is optionally further substituted with one, two, three, four or more substituents independently selected from alkyl, alkoxy, halo, haloalkoxy, —OH, —NH₂, alkylamino, —COOH, or alkoxycarbonyl.

As defined herein, the term “alkoxy” refers to an —OR radical where R is alkyl as defined above, e.g., methoxy, ethoxy, n-propoxy, iso-propoxy, n-butyl, iso-butyl, tent-butyl and the like.

As defined herein, the term “alkoxycarbonyl” or “ester” refers to a —C(O)OR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, or poly(ethylene glycol), and wherein R is optionally further substituted with one, two, three, four or more substituents independently selected from alkyl, alkoxy, halo, haloalkoxy, —OH, —NH₂, alkylamino, —COOH, or alkoxycarbonyl.

As defined herein, the term “alkylamino” refers to an —NHR radical where R is alkyl as defined above, e.g. methylamino, ethylamino, n-propylamino, iso-propylamino, and the like.

As defined herein, the term “aryl” refers to a monovalent monocyclic or bicyclic aromatic hydrocarbon radical of 6 to 10 ring atoms, e.g. phenyl or naphthyl, and the like.

As defined herein, the term “aralkyl” refers to an -(alkylene)-R radical where R is aryl as defined above.

As defined herein, the term “crystal” refers to a slaid material whose constituents (such as atoms, molecules or ions) are arranged in a highly ordered microscopic structure, forming a lattice that extends in all directions. Typically, macroscopic crystals have geometric shapes that consist of flat faces with specific, characteristic orientations.

As defined herein, the term “cycloalkyl” refers to a cyclic saturated monovalent hydrocarbon radical of three to ten carbon atoms wherein one or two carbon atoms may be replaced by an oxo group, e.g. cyclopropyl, cyclobutyl, cyclopentyl, or cyclohexyl, and the like.

As defined herein, the term “cycloalkylalkyl” refers to an -(alkylene)-R radical where R is cycloalkyl as defined above, e.g. cyclopropylmethyl, cyclobutylmethyl, cyclopentylethyl, or cyclohexylmethyl, and the like.

As defined herein, the term “diameter” of a particle refers to the longest linear distance from one side of the particle to the opposite side of the particle, passing through the centre point of the particle. In a method of preparing formulations of the present invention, particles are separated on the basis of their size using a sieving method: those particles with a diameter greater than or smaller than particular critical mesh sizes are excluded from the final formulation.

As defined herein, the term “d₁₀” refers to the mass median diameter of a sample of particles, i.e. the diameter at which 10% of the sample mass is comprised of particles having a diameter less than this value. Typically, d₁₀ values are measured by laser diffraction.

As defined herein, the term “d₅₀” refers to the mass median diameter of a sample of particles, i.e. the diameter at which 50% of the sample mass is comprised of particles having a diameter less than this value. Typically, d₅₀ values are measured by laser diffraction.

As defined herein, the term “d₉₀” refers to the mass median diameter of a sample of particles, i.e. the diameter at which 90% of the sample mass is comprised of particles having a diameter less than this value. Typically, d₉₀ values are measured by laser diffraction.

As defined herein, the term “granule” refers to an aggregate mass comprising smaller particles, which is held together as a discrete structure but in which the original particles may still be separably identified (e.g. by light or electron microscopy). The terms “granule” and “granulate” may be used interchangeably throughout this specification.

As defined herein, the term “halo” refers to fluoro, chloro, bromo, or iodo, preferably fluoro or chloro.

As defined herein, the term “haloalkyl” refers to an alkyl radical as defined above, which is substituted with one or more halogen atoms, preferably one to five halogen atoms, preferably fluorine or chlorine, including those substituted with different halogens, e.g. —CH2Cl, —CF₃, —CHF₂, —CH₂CF₃, —CF₂CF₃, —CF(CH₃)₂, and the like.

As defined herein, the term “haloalkoxy” refers to an —OR radical where R is haloalkyl as defined above, e.g. —OCF₃, —OCHF₂, and the like.

As defined herein, the term “heteroaryl” refers to a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

As defined herein, the term “heteroaralkyl” refers to an -(alkylene)-R radical where R is heteroaryl as defined above.

As defined herein, the term “heterocycyl” refers to a saturated or unsaturated monovalent monocyclic group of 4 to 8 ring atoms in which one or two ring atoms are heteroatoms selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds, provided that the ring is not aromatic.

As defined herein, the term “heterocycloalkyl” refers to an -(alkylene)-R radical where R is heterocyclyl ring as defined above, e.g. tetraydrofuranylmethyl, piperazinylmethyl, morpholinylethyl, and the like.

As defined herein, the term “oral cavity” refers to the cavity of the mouth, and includes the inner upper and lower lips, all parts of the inner cheek, the sublingual area under the tongue, the tongue itself, as well as the upper and lower gums and the hard and soft palate.

As defined herein, the term “pellet” refers to a compact multiparticulate solid mass formed by the agglomeration of finer solid particles (e.g. a powder). This process is referred to as pelletization.

As defined herein, the term “straw” refers to a pipe that enables a user to conveniently consume a liquid. Such straws may be used for oral administration of liquid soluble or liquid insoluble ingredients, preferably granules, which are pre-filled within the straw. To administer the ingredient which is pre-filled in the straw, one end of the pipe is positioned in the liquid and the other end of the pipe is inserted into the oral cavity of the user, and suction is applied to the pipe by the user. Sucked liquid travels through the straw towards the oral cavity of the user, flushing the ingredient through the straw and thus delivering the ingredient to the user via oral administration. Preferably, the ingredient that is pre-filled in the straw is an active pharmaceutical agent (API), food supplement or vitamin.

As defined herein, the term “therapeutically effective amount” refers to an amount of the API which is sufficient to reduce or ameliorate the severity, duration, progression, or onset of a disorder being treated, prevent the advancement of a disorder being treated, cause the regression of, prevent the recurrence, development, onset or progression of a symptom associated with a disorder being treated, or enhance or improve the prophylactic or therapeutic effect(s) of another therapy. The precise amount of API administered to a patient will depend on the type and severity of the disease or condition and on the characteristics of the patient, such as general health, age, sex, body weight and tolerance to drugs. It will also depend on the degree, severity and type of the disorder being treated. The skilled artisan will be able to determine appropriate dosages depending on these and other factors.

As defined herein, the terms “treat”, “treatment” and “treating” refer to the reduction or amelioration of the progression, severity and/or duration of a disorder being treated, or the amelioration of one or more symptoms (preferably, one or more discernible symptoms) of a disorder being treated resulting from the administration of a formulation according to the invention to a patient.

For the avoidance of doubt, all alternative and preferred features relating to the formulation per se apply equally to the use of said formulation in the treatment of a human patient.

### Formulations of the Present Invention

In a first aspect, the present invention is concerned with a pharmaceutical formulation suitable for use in a straw suitable for oral administration of said pharmaceutical formulation, wherein the pharmaceutical formulation is solid and comprises granules, and the granules comprise:
(a) a core comprising an active pharmaceutical ingredient (API), food supplement or vitamin;
(b) optionally, a first coating layer surrounding the core; and
(c) a second coating layer surrounding the first coating layer and/or the core, the second coating layer comprising particles of a sugar, a sugar alcohol, or any mixture thereof.

When the first coating layer is present, the second coating layer surrounds the first coating layer. Thus, the second coating layer surrounds both the first coating layer and the core. When the first coating layer is absent, the second coating layer surrounds the core. Preferably, the first coating layer is present. Alternatively, though, the first coating layer may be absent.

The pharmaceutical formulation according to the present invention is a solid and comprises granules. Thus, the pharmaceutical formulation is not a liquid or gaseous formulation. Granules are as defined herein. Typically, the pharmaceutical formulation is in a form that can be conveyed by a fluid passing through a straw. Preferably, the pharmaceutical formulation comprises granular particles which are spherical, or substantially spherical, in shape. Alternatively, the pharmaceutical formulation consists of, or consists essentially of, granular particles which are spherical, or substantially spherical, in shape.

Typically, the pharmaceutical formulation has a distinct particle size distribution such that at least 80% of the particles in the pharmaceutical formulation by number have a diameter of greater than 200 μm, preferably greater than 250 μm, more preferably greater than 300 μm, and most preferably greater than 400 μm. Preferably, at least 90% of the particles in the pharmaceutical formulation by number have a diameter of greater than 200 μm, preferably greater than 250 μm, more preferably greater than 300 μm, and most preferably greater than 400 μm. More preferably, at least 95% of the particles in the pharmaceutical formulation by number have a diameter of greater than 200 μm, preferably greater than 250 μm, more preferably greater than 300 μm, and most preferably greater than 400 μm.

Further, typically, at least 80% of the particles in the pharmaceutical formulation by number have a diameter of less than 1400 μm, preferably less than 1200 μm, and most preferably less than 1100 μm. Preferably, at least 90% of the particles in the pharmaceutical formulation by number have a diameter of less than 1400 μm, preferably less than 1200 μm, and most preferably less than 1100 μm. More preferably, at least 95% of the particles in the pharmaceutical formulation by number have a diameter of less than 1400 μm, preferably less than 1200 μm, and most preferably less than 1100 μm.

Typically, at least 80% of the particles in the pharmaceutical formulation by number have a diameter of from 200 μm to 1400 μm, preferably from 250 μm to 1400 μm, more preferably from 300 μm to 1200 μm, and most preferably from 400 μm to 1100 μm. Preferably, at least 90% of the particles in the pharmaceutical formulation by number have a diameter of from 200 μm to 1400 μm, preferably from 250 μm to 1400 μm, preferably from 300 μm to 1200 μm, and most preferably from 400 μm to 1100 μm. More preferably, at least 95% of the particles in the pharmaceutical formulation by number have a diameter of from 200 μm to 1400 μm, preferably from 250 μm to 1400 μm, preferably from 300 μm to 1200 um, and most preferably from 400 μm to 1100 μm.

Alternatively, the pharmaceutical formulation may have a distinct particle size distribution such that at least 80% of the particles in the pharmaceutical formulation by volume have a diameter of greater than 200 μm, preferably greater than 250 μm, more preferably greater than 300 μm, and most preferably greater than 400 μm. Preferably, at least 90% of the particles in the pharmaceutical formulation by volume have a diameter of greater than 200 μm, preferably greater than 250 μm, more preferably greater than 300 μm, and most preferably greater than 400 μm. More preferably, at least 95% of the particles in the pharmaceutical formulation by volume have a diameter of greater than 200 μm, preferably greater than 250 μm, more preferably greater than 300 μm, and most preferably greater than 400 μm.

Further, at least 80% of the particles in the pharmaceutical formulation by volume may have a diameter of less than 1400 μm, preferably less than 1200 μm, and most preferably less than 1100 μm. Preferably, at least 90% of the particles in the pharmaceutical formulation by volume have a diameter of less than 1400 μm, preferably less than 1200 μm, and most preferably less than 1100 μm. More preferably, at least 95% of the particles in the pharmaceutical formulation by volume have a diameter of less than 1400 μm, preferably less than 1200 μm, and most preferably less than 1100 μm.

Further, at least 80% of the particles in the pharmaceutical formulation by volume may have a diameter of from 200 μm to 1400 μm, preferably from 250 μm to 1400 μm, more preferably from 300 μm to 1200 μm, and most preferably from 400 μm to 1100 μm. Preferably, at least 90% of the particles in the pharmaceutical formulation by volume have a diameter of from 200 μm to 1400 μm, preferably from 250 μm to 1400 μm, preferably from 300 μm to 1200 μm, and most preferably from 400 μm to 1100 μm. More preferably, at least 95% of the particles in the pharmaceutical formulation by volume have a diameter of from 200 μm to 1400 μm, preferably from 250 μm to 1400 μm, more preferably from 300 μm to 1200 μm, and most preferably from 400 μm to 1100 μm

Alternatively, the pharmaceutical formulation may have a distinct particle size distribution such that at least 80% of the particles in the pharmaceutical formulation by mass have a diameter of greater than 200 μm, preferably greater than 250 μm, more preferably greater than 300 μm, and most preferably greater than 400 μm. Preferably, at least 90% of the particles in the pharmaceutical formulation by mass have a diameter of greater than 200 um, preferably greater than 250 μm, more preferably greater than 300 μm, and most preferably greater than 400 μm. More preferably, at least 95% of the particles in the pharmaceutical formulation by mass have a diameter of greater than 200 μm, preferably greater than 250 μm, more preferably greater than 300 μm, and most preferably greater than

Further, at least 80% of the particles in the pharmaceutical formulation by mass may have a diameter of less than 1400 μm, preferably less than 1200 μm, and most preferably less than 1100 μm. Preferably, at least 90% of the particles in the pharmaceutical formulation by mass have a diameter of less than 1400 μm, preferably less than 1200 μm, and most preferably less than 1100 μm. More preferably, at least 95% of the particles in the pharmaceutical formulation by mass have a diameter of less than 1400 μm, preferably less than 1200 μm, and most preferably less than 1100 μm.

Further, at least 80% of the particles in the pharmaceutical formulation by mass may have a diameter of from 200 μm to 1400 μm, preferably from 250 μm to 1400 μm, more preferably from 300 μm to 1200 μm, and most preferably from 400 μm to 1100 μm. Preferably, at least 90% of the particles in the pharmaceutical formulation by mass have a diameter of from 200 μm to 1400 μm, preferably from 250 μm to 1400 μm, more preferably from 300 μm to 1200 μm, and most preferably from 400 μm to 1100 μm. More preferably, at least 95% of the particles in the pharmaceutical formulation by mass have a diameter of from 200 μm to 1400 μm, preferably from 250 μm to 1400 μm, more preferably from 300 μm to 1200 μm, and most preferably from 400 μm to 1100 μm.

Typically, the d₁₀ value of the particles in the pharmaceutical formulation when measured by laser diffraction is from 200 μm to 500 μm, preferably from 250 μm to 400 μm, and most preferably about 300 μm. Typically, the d₉₀ value of the particles in the pharmaceutical formulation when measured by laser diffraction is from 500 μm to 1000 μm, preferably from 600 μm to 800 μm. Typically, the d90 value of the particles in the pharmaceutical formulation when measured by laser diffraction is from 1000 μm to 1400 μm, preferably from 1100 μm to 1300 μm, and most preferably about 1200 μm. The skilled person, having regard for the desired administration volume for a given application, will be able to select a suitable particle size distribution by simply preparing formulations having a range of different particle size distributions and testing the resultant formulations to measure the volume of liquid required to completely administer the formulation to the subject in need thereof.

A pharmaceutical formulation according to the invention comprises an API, food supplement or vitamin. Thus, typically, a pharmaceutical formulation according to the invention comprises an API. Alternatively, a pharmaceutical formulation according to the invention comprises a food supplement. Alternatively, a pharmaceutical formulation according to the invention comprises a vitamin. Preferably, a pharmaceutical formulation according to the invention comprises an API.

The API, food supplement or vitamin should be physiologically acceptable and compatible with the formulation. Typically, the pharmaceutical formulation comprises from 0.0001% to 99.9% by weight of the API, food supplement or vitamin, preferably from 0.001% to 95% by weight of the API, food supplement or vitamin, more preferably from 0.1% to 90% by weight of the API, food supplement or vitamin, even more preferably from 0.5% to 75% by weight of the API, food supplement or vitamin, yet more preferably from 1% to 60% by weight of the API, food supplement or vitamin, still more preferably from 2 to 50% by weight of the API, food supplement or vitamin, further preferably from 3 to 40% by weight of the API, food supplement or vitamin, and most preferably from 5 to 30% by weight of the API, food supplement or vitamin, e.g. about 10% by weight, about 15% by weight, about 20% by weight, or about 25% by weight of the API, food supplement or vitamin. The API, food supplement or vitamin may be water-soluble. Alternatively, the API, food supplement or vitamin may be poorly soluble in water.

A formulation according to the invention typically comprises one or more pharmaceutically acceptable excipients selected from carriers, diluents, binders, glidants, release control agents and disintegrants. The pharmaceutically acceptable excipients may therefore comprise a mixture of carriers, diluents, binders, glidants, release controlling agents and/or disintegrants. Typically, if the formulation comprises one or more pharmaceutically acceptable excipients, the one or more pharmaceutically acceptable excipients are present in the core of the granules. As an exception, any release control agents are typically incorporated into the formulation as a first coating layer around the surface of the core.

Typically, the pharmaceutical formulation comprises from 0.05% to 99.9% by weight of the one or more pharmaceutically acceptable excipients selected from carriers, diluents, binders, glidants, release controlling agents and disintegrants, preferably from 1% to 99% by weight, more preferably from 5% to 95% by weight, and most preferably from 10% to 90% by weight.

Typically, therefore, the pharmaceutical formulation according to the present invention comprises from 0.0001% to 99.9% by weight of the API, food supplement or vitamin and from 0.05% to 99.9% by weight of the one or more pharmaceutically acceptable excipients selected from carriers, diluents, binders, glidants, release controlling agents and disintegrants. Preferably, the pharmaceutical formulation comprises from 0.001% to 95% by weight of the API, food supplement or vitamin and from 1% to 99% by weight of the one or more pharmaceutically acceptable excipients selected from carriers, diluents, binders, glidants, release controlling agents and disintegrants. More preferably, the pharmaceutical formulation comprises from 0.5% to 75% by weight of the API, food supplement or vitamin and from 5% to 95% by weight of the one or more pharmaceutically acceptable excipients selected from carriers, diluents, binders, glidants, release controlling agents and disintegrants. Most preferably, the pharmaceutical formulation comprises from 2% to 50% by weight of the API, food supplement or vitamin and from 10% to 90% by weight of one or more pharmaceutically acceptable excipients selected from carriers, diluents, binders, glidants, release controlling agents and disintegrants.

### (a) Features of the Core of the Granules

The core of the granules comprises the API, food supplement or vitamin. Thus, typically, the core comprises an API. Alternatively, the core comprises a food supplement. Alternatively, the core comprises a vitamin. Preferably, the core comprises an API. The core may additionally comprise one or more pharmaceutically acceptable excipients selected from carriers, diluents, binders, glidants, release controlling agents and disintegrants, such as those defined herein.

Typically, the API is selected from 13 C-urea (Helicobacter test), 15-Methyl-prostaglandin F2α, 1α-Hydroxyvitamin D3, 2,4-dichlorbenzylalkohol, 5-aminolevulinic acid hydrochloride, 5-aminolevulinsyre (5-ALA), abacavir, abacavir/lamivudine, abacavir/lamivudine/zidovudine, abatacept, abciximab, acamprosat, acarbose, acebutolol, acepromazin, acetaminofene, acetate, acetazolamide, acetophenazine, acetylcysteine, acetylsalicylic acid, aciclovir, acipimox, acitretin, acrivastin, acyclovir, adalimumab, adapalen, adefovir dipivoxil, adenosin, adrenalin, aesculin, agalsidase alfa, agalsidase beta, agalsidase-alfa, agalsidase-beta, agomelatin, agomelatine, alanin, albumin, humant, aldesleukin, alemtuzumab, alendronat, alendronate sodium/colecalciferol, alendronic acid/colecalciferol, alfacalcidol, alfentanil, alfuzosin, alginsyre, alglucosidase alfa, alimemazine, aliskiren, aliskiren hemifumarate/hydrochlorothiazide, alitretinoin, allopurinol, almitrin, almotriptan, alprazolam, alprenolol, alprostadil, alteplase, aluminiumaminoacetat, aluminiumhydroxid, aluminiumsaccharosesulfat, alkalic, amantadine, ambenon, ambrisentan, ambroxol, amfepramon, amidotrizoat, amiloride, aminofyllin, aminogluthetimid, aminosalyl, amiodaron, amisulprid, amitriptylin, amlodipin, amlodipine besylate/valsartan/hydrochlorothiazide, amlodipine besylate/valsartan, amlodipine/valsartan, amorolfin, amoxicillin, amphotericin B, ampicillin, amprenavir, amsachrin, amylase, amylmetacresol, anagrelide, anakinra, anastrozol, anidulafungin, antazoline, antithrombin, antithrombin alfa, anti-thymocytglobulin, apomorphine, apraclonidin, aprepitant, aprotinin, arcitumomab, argatroban, arginin, aripiprazole, arsenic trioxide, articain, ascorbic acid, asparagin, atazanavir, atenolol, atomoxetin, atorvastatin, atosiban, atovaquon, atropine, auranofin, aurothiomalat, aviptadil, azacitidin, azacitidine, azapropazone, azathioprin, azelaic acid, azelastine, azetazolamide, azithromycin, aztreonam, aztreonam C1-esterase-inhibitor, human, bacampicillin, bacillus Calmette Guérin (Danish strain 1331), bacillus Calmette Guérin (strain RIVM derived from strain 1173-P2), baclofen, balsalazid, bambuterol, bariumsulfat, basiliximab, bazedoxifene, becaplermin, bechlomethasone, beclometasondipropionat, benazepril, bendroflumethiaziede, bensatropine, benserazid, benzylpenicillin, benzalkonium chloride, benzene carboxylic acid, benzenmethanol, benzocain, benzoic acid, benzoylperoxid, benzydamin, benzylpenicillin, betacarotene, betahistin, betain, betaine anhydrous, betamethason, betamethason-17-valerat, betamethason-21-acetat, betamethasondipropionat, betamethasonphosphat, betanidine, betaxolol, bevacizumab, bexarotene, bicalutamid, bimatoprost, bimatoprost/timolol, biotin, biperiden, bisachodyl, bisoprololfumarat, bivalirudin, black rubber-mix (PPD-mix), bleomycin, borax, bortezomib, bosentan, botulinum toxin type a, botulinum toxin type B, brimonidin, brimonidintartrat, brinzolamide, brinzolamide/timolol, bromazepam, bromhexine, bromocriptine, brompheniramine, budesonide, bumetanide, butamirate citrate, bupivacain, buprenorphine, buprenorphine/naloxone, bupropion, buserelin, buspiron, busulfan, butylscopolamin, cab ergolin, cadexomer-iodine, caffeine, cain-mix, calcipotriol, calcitirol, calcitonin, calcitonin (salmon), calcium, calciumacetate, calciumcarbonate, calciumchloride, calciumfluoride, calciumfolinate, calciumgluconate, calciumlactogluconate, calciumpolystyrensulfonate, canakinumab, candesartancilexetil, capecitabine, capsaicin, captopril, carbamazepine, carba-mix, carbetocin, carbidopa, carbimazol, carbomer, carbocistein, arbon, active, carboplatin, carboprost, carglumic acid, carmelloseSodium, carmustin, carvedilol, caspofungin, catumaxomab, cefalexin, cefotaxim, cefoxitin, cefprozil, ceftazidim, ceftriaxon, cefuroxim, celecoxib, cephaclor, cephadroxil, cephalexin, cephalotin, cephradin, certolizumab pegol, cetirizin, cetrorelix, cetuximab, chinidine, chlofibrate, chlomethiazol, chlomipramin, chlonazepam, chloprothixene, chloralhydrat, chlorambucil, chloramphenicol, chlordiazepoxid, chlorhexidine, chloride, chloriongonadotropin, chloroquin, chlorpromazine, chlorpropamid, chlorprothixen, chlorthalidon, chlorzoxazon, chlotrimazol, cholecalciferol, vitamin D3, cholinetheophyllinate, choriogonadotropin alfa, choriongonadotropin, humant (hCG), choriongonadotropin-α (hCG), chrome, ciclopirox, ciclopiroxolamin, ciclosporin, cidofovir, cilastatin, cimetidine, cinacalcet, cinchocain, cinetazon, cinnamaldehyd, cinnamylalcohol, cinnarizine, ciprofloxacin, cis(Z)-flupenthixoldecanoat, cisatracurium, cisplatin, citalopram, Cl+Me-isothiazolinon (Kathon CG), cladribin, cladribine, clarithromycin, clavulansyre, clemastin, clemastine, clindamycin, clioquinol, clobazam, clobetasolpropionat, clobetason-17-butyrat, clodronat, clofarabin, clomiphene, clomipramin, clonazepam, clonidine, clopamide, clopidogrel, clotrimazol, cloxacillin, clozapin, cobalt(II), cobber, cobber acetate, codeine, colesevelam, colestipol, colestyramin, coli stimethatSodium, corticotropin, cortisone, cyanochobalamine, cyanocobalamin, vitamin B12, cyclandelar, cyclizine, cyclopentolat, cyclophenile, cyclophosphamid, cyproheptadine, cyproteron, cyproteronacetat, cysteamin, cystein, cystin, cytarabin, cytarabine, dabigatran etexilate, dacarbazine, daclizumab, dalteparin, dantron, dapson, daptomycin, darbepoetin alfa, darifenacin, darifenacin, darunavir, dasatinib, daunorubicin, deferasirox, deferiprone, deferoxaminmesilat, degarelix, demeclocycline, depreotide, desfluran, desipramin, desirudin, deslanoside, desloratadine, desloratadine (as sulphate), desmopres sin, desogestrel, desoximethason, dexamethason, dexchlorpheniramine, dexibuprofen, dexketoprofen, dexpantenol, dexpanthenol, Vitamin B5, dexrazoxane, dextran 1, dextran 40, dextran 70, dextromethorphan, dextropropoxyphene, diazepam, diazoxide, dibotermin alfa, dichlophenamide, diclofenac, diclofenacSodium, dicloxacillin, diculmarole, didanosin, dienogest, digoxine, dihydralazine, dihydroergotamine, dihydrogesteron, dihydrotachysterol, dihydroxyaluminium sodiumcarbonat, dikaliumchlorazepat, diltiazem, dimeglumingadopentetat, dimenhydinate, dimethylaminodiphenylbuten, dimeticon, dimeticon, ferrofumarate, dinitrogenoxid, dinoprost, dinoproston, diosmin, diphenhydramin, diphenolxylate, dipyradamol, diSodiumclodronate, diSodiumetidronate, diSodiumphosphate, disopyramide, disulfiram, dixyrazine, dobutamine, docetaxel, docosahexaenoinsyre (DHA), docusat, dofetilide, domperidon, donepezil, dopamine, doripenem, dornase alfa, dorzolamid, dosulepin, doxapram, doxazosin, doxepin, doxorubicin, doxorubicin hydrochloride, doxycyclin, doxycycline, droperidol, drospirenon, drotrecogin alfa (activated), duloxetine, dutasterid, ebastin, econazol, eculizumab, efalizumab, efavirenz, efavirenz/emtricitabine/tenofovir disoproxil (as fumarate), eflornithine, eicosapentaenoinsyre (EPA), ekonazol, eletriptan, emedastine, emepron, emtricitabine, emtricitabine/tenofovir disoproxil, enalapril, enfuvirtide, enoxaparin, entacapone, entecavir, ephedrine, epinephrine, epirubicin, eplerenon, epoetin alfa, epoetin beta, epoetin delta, epoetin zeta, epoprostenol, epototermin alfa, epoxyresin, eprosartan, eptacog alfa (activated), eptifibatid, eptifibatide, eptotermin alfa, erdostein, ergocalciferol, vitamin D2, ergotamine, erlotinib, erlotinib, ertapenem, erythromycin, escitalopram, eslicarbazepin, eslicarbazepine acetate, esmolol, esomeprazol, estradiol, estradiolvalerat, estradiolvalerianate, estramustin, estramustinphosphat, estriol, etambutol, etanercept, etanercept, ethacrynacide, ethambutol, ethinylestradiol, ethosuximide, ethylendiamin, ethylmorphine, etidronat, etilephrine, etodolac, etonogestrel, etoposide, etoricoxib, etravirin, etravirine, etulos, eugenol, everolimus, exemestan, exenatid, exenatide, ezetimibe, ezlocillin, factor IX, factor VIII, famciclovir, febuxostat, felodipin, felypressin, fenoterol, fentanyl, fentanyl citrate, ferri-salts, ferritetrasemisodium, ferro-salts, ferrosuccinate, ferumoxsil, fesoterodine, fexofenadin, fibrinogen, fibronektin, filgrastim, finasterid, fiskeolie, flavoxat, flecainide, flucloxacillin, fluconazol, flucytosin, fludarabinphosphat, fludrocorti son, fludrocortisonacetat, flumazenil, flumedroxon, flumetasonpivalat, flunarizin, flunitrazepam, fluocinolonacetonid, fluocinonid, fluocortolon 21-pivalat, fluorid, fluormetolon, fluoruracil, fluoxetin, fluoximesteron, flupentizol, fluphenazindecanoat, fluphenazine, flurbiprofen, flutamid, fluticasone furoate, fluticasonpropionat, fluvastatin, fluvoxamin, folic acid, folic acid heparin, follitropin alfa, follitropin beta, follitropin-α (rfSH), follitropin-β (rfSH), fomivirsen, fondaparinux, fondaparinux sodium, formaldehyde, formoterol, fosamprenavir, fosaprepitant, fosaprepitant dimeglumine, fosinopril Sodium, fosphenytoin, framycetin, frangulabark, frovatriptan, fulvestrant, furosemide, fusidic acid, gabapentin, gadobutrol, gadodiamid, gadofosveset, gadoteridol, gadoterinsyre, gadoversetamide, galantamin, galsulfase, ganciclovir, ganirelix, gefitinib, gelatine, gemcitabin, gemeprost, gemfibrozil, gentamicin, geraniol, gestoden, glatirameracetat, glibenclamid, gliclazid, glimepirid, glipizid, glucagon, glucopyrron, glucosamin, glucose, glutamin, glutathion, glycerol, glycerophosphat, glyceryl nitrate, glycerylnitrate, glyceryltrinitrate, glycin, glycopyrron, glycyl-glutamin, glycyl-tyrosin, golimumab, goserelin, gramicidin, granisetron, griseofulvin, guanetidine, guanfacine, haloperidol, hedera helix extract, heparin, heparin co-factor, heparinoid, hesperidin, hexaminolevulinat, histamine, histidine, histrelin, human coagulation factor IX, human fibrinogen/human thrombin, human normal immunoglobulin, human normal immunoglobulin (IVIg), hydralazine, hydrochloride, hydrochlorthiazide, hydrocortisonacetat, hydrocortisone, hydrocortisone-17-butyrat, hydrocortisonsuccinat, hydrogenperoxid, hydromorphon, hydroxichloroquine, hydroxiprogresterone, hydroxizine, hydroxocobalamin, vitamin B12, hydroxycarbamide, hydroxychloroquin, hydroxycitronellal, hydroxyethylrutosider, hydroxyethylstivelse starch, hydroxyurea, hyoscin, hyoscinbutylbromid, hyoscyamine, hypromellose, ibandronic acid, ibandronsyre, ibritumomab tiuxetan, ibuprofen, icatibant, ichthammol, icodextrin, idarubicin, idursulfase, ifosfamid, iloprost, imatinib, imatinib mesilate, imiglucerase, imipenem, imipramin, imiquimod, immunglobulin G, humant, immunglobulin, humant (anti-D), indapamid, indinavir, indomethacine, infliximab, inositolnico-tinate, insulin, insulin aspart, insulin aspart protamin, insulin detemir, insulin glargine, insulin glulisine, insulin human (rDNA), insulin lispro, insulin lispro protamin, insulin, humant, insulin, isophan, humant, interferon alfa-2b, interferon alfacon-1, interferon beta-1a, interferon idoxuridin, interferon-alfa, interferon-alfa-2b, interferon-beta-1a, interferon-beta-1b, interferon-gamma-1b, interleukin-2, iobitridol, iodidine, iodixanol, ioflupane (123 I), iohexol, iomeprol, iopromid, iotrolan, ioversol, ipratropium, irbesartan, irbesartan/hydrochlorothiazide, irinotecan, isocarboxazid, isoeugenol, isofluran, isoleucin, isoniazid, isophaninsulin, humant, isoprenaline, isosorbiddinitrate, isosorbidmononitrate, isotretinoin, isradipin, itraconazol, ivabradine, ketobemidon, ketobemidone, ketokonazol, Ketoprofen, ketorolac, ketotifen, Kolofon, kreatinin monohydrate, kreatinin monohydrate, labetalol, lacidipin, lacosamide, lactat, lactic acid, lactic acid producing bacteria, lactulose, lamivudine, lamivudine/zidovudine, lamotrigin, lanolin, lanreotid, lansoprazol, lanthanum, lapatinib, laronidase, laropiprant, lasofoxifene, latanoprost, lecithin, leflunomide, lenalidomide, lenograstim, lepirudin, lercanidipin, letrozol, leucin, leucovorin, leuprorelin, levetiracetam, levocabastin, levocetirizin, levodopa, levofloxacin, levofolic acid, levomepromazine, levonorgestrel, levotyroxin, lidocain, lincomycin, linezolid, liotyronin, lipase, liraglutide, lisinopril, lithiumcarbonat, lithiumcitrat, lodoxamid, lofepramin, lomustine, loperamide, lopinavir, loratadin, lorazepam, lormetazepam, lornoxicam, losartan, lovastatin, lutropin alfa, lymecycline, lynestrenol, lypressin, lysine, macrogol 3350, magnesium, magnesium carbonate, magnesium chloride, magnesium hydroxide, magnesiumoxide, magnesiumsulfate, malathion, mangafodipir, mangane, mannitol, maptrotilin, maraviroc, mebendazol, mebeverin, mecasermin, mecillinam, meclozine, medroxiprogresterone, medroxyprogesteronacetate, mefloquine, mefruside, megesterol, megestrolacetat, melatonin, melfalan, meloxicam, melperon, melphalan, memantine, meningokokpolysaccharid, menotropin (hmG), mepensolar, mepivacain, meprobamat, mepyramin, mercaptamine bitartrate, mercaptobenzothiazol, mercapto-mix, mercaptopurin, meropenem, mesalazin, mesna, mesterolon, mestranol, metacycline, metaoxedrin, metenamine, metformin, meth ldopa, methadone, methenamin, methionin, metholazone, methotrexat, methoxy polyethylene glycol-epoetin beta, methyl aminolevulinat, methyldopa, methylergometrin, methylergotamine, methylnaltrexon, methylnaltrexone bromide, methylperon, methylphenidat, methylprednisolon, methylprednisolonacetat, methylprednisolonsuccinat, methyl scopolamine, methyprylon, metixene, metoclopramide, metopimazin, metoprolol, metronidazole, metychlothiazide, mexiletin, mianserin, micafungin, miconazole, midazolam, mifamurtide, miglustat, minoxidil, mirtazapin, misoprostol, mitomycin, mitotane, mitoxantron, mivacurium, moclobemid, modafinil, molybdenum, mometasonfuroat, moroctocog alfa, morphine, moxaverine, moxifloxacin, moxonidin, mupirocin, mycophenoic acid, mycophenolate mofetil, nabumeton, nadolol, nafarelin, nalbuphin, nalidixic acid, naloxone, naltrexon, nandrolon, naphazolin, naproxen, naratriptan, natalizumab, natamycine, nateglinide, nebivolol, nelarabin, nelarabine, nelfinavir, neomycin, neomycinsulfat, neostigmin, nepafenac, nevirapine, nicheritrol, nickel, nicomorphin, nicorandil, nicotin, nicotinamid, nicotinic acid, nicotinic acid/laropiprant, nicotinyl alchol, nifedipine, nilotinib, nimodipin, niphedipin, nitisinone, nitrazepam, nitrendipin, nitric oxide, nitrofurantoin, nitrogen, nitrogen oxide, nitroprus side, nizatidin, nonacog alfa, noradrenalin, norelgestromin, norelgestromin/ethinyl estradiol, norethisteronacetat, noretisterone, norfloxacin, norgestimat, nortriptylin, noscapine, nystatin, oak moss, octocog alfa, octreotid, ofloxacin, olanzapine, olmesartanmedoxomil, olopatadine, olsalazin, omalizumab, omeprazol, ondansetron, opipramol, opium, oral Cholera vaccine, orciprenaline, orlistat, ornidazol, ornithin, orphenadrine, oseltamivir, osteogent protein-1: BMp-7, oxaliplatin, oxazepa, oxazepam, oxcarbazepin, oximetolon, oxiphencyclimine, oxitetracycline, oxprenolol, oxybutynin, oxycodon, oxygen, oxymetazolin, oxytetracyclin, oxytocin, paclitaxel, paclitaxel albumin, palifermin, palifermin, paliperidone, palivizumab, palonosetron, pamidronat, panitumumab, pantoprazole, pantotenol, vitamin B5, pantothenic acid, papaverine, paracetamol, paraffinolie, parathyroid hormone (rDNA), parecoxib, paricalcitol, paroxetin, pegaptanib, pegaptanib sodium, pegfilgrastim, peginterferon alfa-2a, peginterferon alfa-2b, pegvisomant, pegylated interferon-alfa-2a, pegylated interferon-alfa-2b, pemetrexed, penciclovir, penfluridol, penicillamine, pentaeritrityltetranitrate, pentazocine, pentobarbital, pentoxifyllin, pentoxiverine, perflutren, pergolid, periciazin, perindopril, permethrin, perphenazindecanoat, perphenazine, pertussistoksoid, pethidin, pethidine, phenazone, phenazonsalicylat, phenemal, phenfluramin, phenobarbital, phenoperidine, phenoxymethylpenicillin, phenprocoumon, phentanyl, phentolamin, phenylamine, phenylbutazone, phenylephrin, phenylpropanolamine, phenytoine, phosphat, phosphestrol, phytomenadion, vitamin K1, phytominadion, pilocarpin, pimecrolimus, pimozid, pindolol, pioglitazone, pioglitazone/glimepiride, pioglitazone/metformin, pioglitazone/metformin hydrochloride, pipamperon, piperacillin, piritramide, piroxicam, pivampicillin, pivmecillinam, pizitifen, pizotifen, plasminogen, plerixafor, podophyllotoksin, polydocanol, polyestradiolphosphat, polygelin, polymyxin B, polythiazide, posaconazole, potassium, potassium acetate, potassium chloride, potassium dihydrogen phosphate, potassium dikromat, potassium hydroxide, potassium phosphate, p-phenylendiamin, pramipexole, prasugrel, pravastatin, prazosine, prednisolon, prednisolon sodiumphosphate, prednisone, pregabalin, prenalterol, prilocain, primidone, probanteline, probenecid, procain, procainamide, procarbazine, prochlorperazine, procylidine, proetazine, progesteron, proguanil, prolin, promethazine, propafenon, propanthelinbromid, propionmazine, propofol, proproanolol, propylthiouracil, propyphenazon, proscillaridin, protamin, protein C, protein C, human, protein S, protriptylin, proxiphylline, prucalopride, pseudoephedrine (as sulphate), p-t-butylphenol-formaldehyd-resin, pyrazinamid, pyridostigmine, pyridoxin, pyridoxin, vitamin B6, pyrityldion, pyrvin, quetiapin, quinagolid, quinapril, quinin, quinolin-mix, rabeprazol, raffinose, raloxifene, raltegravir, ramipril, ranibizumab, ranitidine, ranolazine, rasagiline, rasburicase, reboxetin, recombinant human erythropoietin alfa, remifentanil, repaglinide, reserpine, resorcinol, retapamulin, reteplase, retinol, retinol, vitamin A, ribavirin, riboflavin, vitamin B2, rifabutin, rifampicin, riiterol, rilonacept, riluzole, rimexolon, rimonabant, risedronat, risperidon, ritonavir, rituximab, rivaroxaban, rivastigmine, rizatriptan, rocuronium, romiplostim, ropinirol, ropivacain, rosiglitazone, rosiglitazone/glimepiride, rosiglitazone/metformin, rosuvastatin, rotavirus, rotigotine, roxithromycin, rufinamide, sagradaextract, salazosulfapyridin, salazosulfapyridine, salbutamol, salicylic acid, salicylic amide, salmeterol, samarium [153sm]lexidronam pentasodium, sapropterin, saquinavir, saxagliptin, scopolamine, selegilin, selenium, selenium disulfid, sennaglycosides, serin, sertindol, sertralin, sevelamer, sevelamer (carbonate), sibutramin, sildenafil, simeticon (aktiveret dimeticon), simvastatin, sirolimus, sitagliptin, sitagliptin/metformin hydrochloride, sitagliptin phosphate monohydrate/metformin hydrochloride, sitaxentan, sitaxentan sodium, s-ketamin, sodium oxybate, sodium phenylbutyrate, sodium-chromoglicate, sodiummaurothiomalate auronofin, sodiumpicosulfat, solifenacin, sølvsulfadiazin, somatotropin, somatrem, somatropin, sorafenib, sorbitol, sotalol, spectinomycin, spiramycin, spironolactone, stanozolol, stavudine, stiripentol, streptokinase, strontium ranelate, sucralfat, sufentanil, sugammadex, sulbentin, sulesomab, sulfamethizol, sulfamethoxazol, sulfasalazin, sulfat, sulfisomidine, sulphur hexafluoride, sulpirid, sumatriptan, sunitinib, suxamethon, synstigmine, tacrolimus, tadalafil, tafluprost, tamoxiphene, tamsulosin, tasonermine, taurin, tazobactam, tegafur, teicoplanin, telbivudine, telithromycin, telmisartan, telmisartan/hydrochlorothiazide, temoporfin, temozolomide, temsirolimus, tenecteplase, teniposide, tenofovir disoproxil, tenoxicam, terazosin, terbinafin, terbutalin, teriparatide, terlipres sin, terodiline, testosterone, testosteronenantat, testosteronundecanoat, tetanustoksoid, tetrabenazin, tetracosactid, tetracycline, tetryzolin, thalidomide, theophlline, theophyllin og ethylendiamin, thiamazol, thiamin, vitamin B 1, thiethylperazine, thioguanine, thiomersal, thiopental, thioridazine, thiotepa, thithixen, threonin, thrombin, human, thyrotropin alfa, tiagabin, tiamazol, tiamin, tiaprofenic acid, tibolon, tigecyclin, tigecycline, timolol, tinidazole, tinzaparin, tiotropium, tipranavir, titandioxide, tizanidin, tobramycin, tocilizumab, tocofersolan, tocopherol, vitamin E, tokoferol, tolazamid, tolbutamid, tolcapone, tolfenamic acid, tolterodin, tolvaptan, topiramat, topotecan, toremifene, trabectedin, tramadol, trandolapril, tranexamic acid, trastuzumab, travoprost, travoprost, travoprost/timolol, treosulfan, treprostinil, triacelluvax, triamcinolonacetonid, triamcinolonhexacetonid, triazolam, trifluoperazine, triglycerid, trimetazidin, trimethaphan, trimethoprim, trimipramin, triptorelin, trombin, tropicamid, tropisetron, trospiumchlorid, tryptophan, tyrotropin, ulipristal, ulipristalacetat, urofollitropin (uFSH), urokinase, ustekinumab, valaciclovir, valdecoxib, valganciclovir, valin, valproat, valsartan, vancomycin, vardenafil, vareniclin, varenicline tartrate, vasopressin, venlafaxin, verapamil, verteporfin, vigabatrin, vildagliptin, vildagliptin/metaformin hydrochloride ldagliptin, vildagliptin/metformin hydrochloride, vinblastin, vinchristin, vindesin, vinflunine ditartrate, vinorelbin, zonisamide, zopiclon, zuclopenthixol, zuclopenthixolacetate, zuclopenthixoldecanoat, zuclopentizol, α1-proteinaseinhibitor (human), α-amyl cinnamaldehyde and pharmaceutically acceptable salts thereof, and mixtures thereof.

The API may also be a prescription or non-prescription substance such as a vaccine. Non-limiting examples of vaccines include viable autologous cartilage cells expanded ex vivo expressing specific marker proteins, combined diptheria, tetanus, acellular pertussis and hepatitis B recombinant vaccine, combined hepatitis A and hepatitis B vaccine, diphtheria, tetanus, pertussis, hepatitis B, Haemophilus influenzae type b conjugate vaccine, Diphtheria, tetanus, whole cell pertussis and hepatitis B vaccine, diptheria, tetanus, acellular pertussis, hepatitis B recombinant (adsorbed), inactivated poliomyelitis and absorbed conjugate haemophilus influenzae type b vaccine, diptheria, tetanus, acellular pertussis, hepatitis B recombinant (adsorbed), inactivated poliomyelitis vaccine, haemophilus b conjugate (Meningoccocal Protein conjugate) and hepatitis B (recombinant) vaccine, hepatitis A (inactivated), hepatitis B(rDNA)(HAB) antigen vaccine (adsorbed), hepatitis B (rDNA) vaccine (adjuvanted, adsorbed), hepatitis B (Recombinant) Vaccine, human papillomavirus vaccine, human papillomavirus vaccine [types 6, 11, 16, 18] (recombinant, adsorbed), human rotavirus, live attenuated, Inactivated Hepatitis A virus HBsAg recombinant purified, influenza vaccine (split virion, inactivated), Influenza vaccine (surface antigen, inactivated, prepared in cell culture), Japanese Encephalitis Vaccine (inactivated, adsorbed), measles, mumps and rubella vaccine (live), measles, mumps, rubella and varicella vaccine (live), Pandemic influenza vaccine, Pandemic influenza vaccine (H1N1) (split virion, inactivated, adjuvanted); A/California/7/2009 (H1N1)v like strain (X-179A), Pandemic influenza vaccine (surface antigen, inactivated, adjuvanted); A/California/7/2009 (H1N1)v like strain (X-179A), pandemic influenza vaccine (whole virion, vero cell derived, inactivated) pneumococcal polysaccharide conjugate vaccine (adsorbed), pneumococcal saccharide conjugated vaccine, absorbed, prepandemic influenza vaccine (H5N1) (split virion, inactivated, adjuvanted) A/Vietnam/1194/2004 NIBRG-14, rotavirus vaccine, and shingles (herpes zoster) vaccine (live).

Typically, the food supplement is selected from retinol, retinyl acetate, retinyl palmitate, beta-carotene, cholecalciferol, ergocalciferol, D-alpha-tocopherol, DL-alpha-tocopherol, D-alpha-tocopheryl acetate, DL-alpha-tocopheryl acetate, D-alpha-tocopheryl acid succinate, phylloquinone (phytomenadione), menaquinone-7, thiamin hydrochloride, thiamin mononitrate, riboflavin, riboflavin 5′-phosphate, sodium, nicotinic acid, nicotinamide, D-pantothenate, calcium, D-pantothenate, sodium, dexpanthenol, pyridoxine hydrochloride, pyridoxine 5′-phosphate, pteroylmonoglutamic acid, calcium-L-methylfolate, (6S)-5-methyltetrahydrofolic acid, glucosamine salt, cyanocobalamin, hydroxocobalamin, D-biotin, L-ascorbic acid, sodium-L-ascorbate, calcium-L-ascorbate, potassium-L-ascorbate, L-ascorbyl 6-palmitate, calcium carbonate, calcium chloridecalcium salts of citric acid, calcium gluconate, calcium glycerophosphate, calcium lactate, calcium salts of orthophosphoric acid, calcium hydroxide, calcium oxide, magnesium acetate, magnesium carbonate, magnesium chloride. magnesium salts of citric acid, magnesium gluconate, magnesium glycerophosphate, magnesium salts of orthophosphoric acid, magnesium lactate, magnesium hydroxide, magnesium oxide, magnesium sulphate, ferrous carbonate, ferrous citrate, ferric ammonium citrate, ferrous gluconate, ferrous fumarate, ferric sodium diphosphate, ferrous lactate, ferrous sulphate, ferric diphosphate (ferric pyrophosphate), ferric saccharate, ferrous ammonium phosphate, ferric sodium EDTA, elemental iron (carbonyl+electrolytic+hydrogen reduced), ferrous bisglycinate, cupric carbonate, cupric citrate, cupric gluconate, cupric sulphate, copper lysine complex, sodium iodide, sodium iodate, potassium iodide, potassium iodate, zinc acetate, zinc chloride, zinc citrate, zinc gluconate, zinc lactate, zinc oxide, zinc carbonate, zinc sulphate, manganese carbonate, manganese chloride, manganese citrate, manganese gluconate, manganese glycerophosphate, manganese sulphate, sodium bicarbonate, sodium carbonate, sodium chloride, sodium citrate, sodium gluconate, sodium lactate, sodium hydroxide, sodium salts of orthophosphoric acic, sodium sulphate, potassium bicarbonate, potassium carbonate, potassium chloride, potassium citrate, potassium gluconate, potassium glycerophosphate, potassium lactate, potassium hydroxide, potassium salts of orthophosphoric acic, potassium sulphate, sodium selenate, sodium hydrogen selenite, sodium selenite, chromium (III) chloride, chromium (III) sulphate, chromium picolinate, chromium(III) lactate tri-hydrate, ammonium molybdate (molybdenum (VI)), sodium molybdate (molybdenum (VI)), potassium fluoride, sodium fluoride, monomethylsilanetriol (organic silicon), activated charcoal, alpha-linolenic acid (ALA), arabinoxylan produced from wheat endosperm barley grain fibre, beta-glucans, betaine, biotin, carbohydrate-electrolyte solutions, chitosan, choline, creatine, docosahexaenoic acid (DHA), eicosapentaenoic acid (EPA), glucomannan, guar gum, hydroxypropyl methylcellulose (HPMC), lactase enzyme, lactulose, linoleic acid, live yoghurt cultures, melatonin, monascus purpureus (red yeast rice), monounsaturated and/or polyunsaturated fatty acids, oat grain fibre, oleic acid, olive oil polyphenols, pectins, plant sterols, plant stanols, resistant starch, rye fibre, xylitol, sorbitol, mannitol, maltitol, lactitol, isomalt, erythritol, sucralose and polydextrose; D-tagatose and isomaltulose, wheat bran fibre, zeaxanthin, lutein, dextrose anhydrous, dextrose monohidrate fructose, glucose, sugar, colostrum, Lactobacillus strains, Bifidobacterium strains, Saccharomyces boulardii, Streptococcus thermophilus, Bacillus laterosporus, Pediococcus acidilactici, Lactococcus lactis, inulin, fructooligosaccharides, galacto-oligosaccharides, soy-oligosaccharides, xylo-oligosaccharides, isomalto-oligosaccharides, and mixtures thereof.

Typically, the vitamin is selected from vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and mixtures thereof.

The core may comprise one or more pharmaceutically acceptable carriers. Typically, the pharmaceutically acceptable carrier is a polymeric carrier. Typically, the carrier is a polymeric carrier selected from gelatins, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polycarbophil, polyethylene glycol (PEG), polyethylene oxides, polyoxyalkylene derivatives, polymethacrylates, poly(vinyl pyrrolidone) (PVP), polyvinyl acetate (PVAc), PVP-vinylacetate-copolymer (PVP-VA), a vinylpyrrolidone-vinyl acetate copolymer (such as Kollidon® VA 64), lactose, sorbitol, mannitol, maltitol, saccharose, isomalt, cyclodextrins such as a-cyclodextrins, β-cyclodextrins, γ-cyclodextrins, hydroxylpropyl-cyclodextrins, hydroxypropyl-β-cyclodextrin (HP-β-CD), sodium carboxymethyl cellulose, sodium alginate, xantham gum, locust bean gum, chitosan, cross-linked high amylase starch, cross-linked polyacrylic acid (carbopol), and mixtures thereof.

The core may comprise one or more pharmaceutically acceptable diluents. Typically, the pharmaceutically acceptable diluent is selected from saccharides (such as monosaccharides, disaccharides, and polysaccharides), sugar alcohols (such as arabinose, lactose, dextrose, sucrose, fructose, maltose, mannitol, erythritol, sorbitol, xylitol, lactitol), powdered cellulose, microcrystalline cellulose, starch, dibasic calcium phosphate, tribasic calcium phosphate, calcium carbonate, dextrose, kaolin, magnesium carbonate, magnesium oxide, purified sugar, and mixtures thereof.

The core may comprise one or more pharmaceutically acceptable binders. Typically, the pharmaceutically acceptable binder is selected from methyl cellulose, hydroxyethyl cellulose, hydroxypropyl cellulose, hydroxypropylmethylcellulose (HPMC), carbomers, dextrin, ethyl cellulose, methylcellulose, shellac, zein, gelatin, polymethacrylates, poly(vinyl pyrrolidone) (such as poly(vinyl pyrrolidone) K30 or poly(vinyl pyrrolidone) F90), starch, pregelatinized starch, polyvinyl alcohol, tragacanth, sodium alginate, gums, synthetic resins, silicic acid, hydrophilic polymers, and mixtures thereof.

The core may comprise one or more pharmaceutically acceptable glidants. Typically, the pharmaceutically acceptable glidant is selected from talc, metallic stearates (such as magnesium stearate, calcium stearate, and zinc stearate), colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin, and mixtures thereof. It would be appreciated that a person skilled in the art is cognizant of the fact that a glidant may also be referred to as either a lubricant or an anti-tacking agent, and that these terms may be used interchangeably.

The core may comprise one or more pharmaceutically acceptable disintegrants. Typically, the pharmaceutically acceptable disintegrant is selected from the group consisting of croscarmellose sodium, crospovidone, sodium starch glycolate, corn starch, potato starch, maize starch and modified starches (such as pregelatinized starch), calcium silicates, low-substituted hydroxypropylcellulose, and mixtures thereof.

In addition to the aforementioned excipients, the core may further comprise one or more additional excipients. Typically, said additional excipients may be selected from pharmaceutically acceptable surfactants, acids, fast-dissolving small molecules, taste-masking agents, flavouring agents, sweeteners, colorants, pore-forming agents, plasticizers, preservatives, and combinations thereof.

Thus, the core may optionally further comprise a surfactant. In particular, a surfactant may be present if the API, food supplement or vitamin is poorly soluble in water. The surfactant, if present, may be dispersed uniformly, or substantially uniformly, throughout the core, i.e. the formulation comprises an intragranular surfactant. Alternatively, the surfactant, if present, may coat the surface of the core, i.e. the formulation comprises an extragranular surfactant. Typically, the surfactant is selected from a cationic surfactant, an anionic surfactant, a non-ionic surfactant, a zwitterionic surfactant, and mixtures thereof.

Suitable cationic surfactants are typically selected from quaternary ammonium compounds (such as benzalkonium chloride, cetyl trimethyl ammonium bromide and dodecyl dimethyl ammonium bromide), hexadecyl (cetyl) trimethylammonium bromide, dodecyl pyridinium chloride, lauryl dimethyl benzyl ammonium chloride, acyl carnitine hydrochlorides, alkyl pyridinium halides, dodecylamine hydrochloride, and mixtures thereof.

Suitable anionic surfactants are typically selected from salts of aliphatic monoesters of sulfuric acid and soaps (such as potassium laurate, sodium dodecyl sulfate, alkyl polyoxyethylene sulfates, sodium alginates, sodium lauryl sulfate and sodium heptadecyl sulfate), sulfonated aromatic agents (such as alkyl benzene sulfonic acids and salts thereof, such as tridecylbenzene sulfonic acid and the sodium and amino salts of dodecylbenzene sulfonic acid), alkyl naphthalene sulfonates (such as sodium butylnaphthalene sulfonate), sulfosuccinates (such as sodium dioctyl sulfosuccinate and N-acyl-N-alkyl fatty acid taurates), sulfated polyoxyethylated alcohols, sulfated oils, dioctyl sodium sulfosuccinate, phosphatidyl choline, phosphatidyl glycerol, phosphatidyl inosine, phosphatidylserine, phosphatidic acid, glyceryl esters, sodium carboxymethylcellulose, cholic acid and other bile acids (such as cholic acid, deoxycholic acid, glycocholic acid, taurocholic acid, and glycodeoxycholic acid), pharmaceutically acceptable salts thereof, and mixtures thereof.

Suitable non-ionic surfactants are typically selected from polyoxyethylene fatty alcohol ethers (such as Macrogol and Brij), polyoxyethylene sorbitan fatty acid esters (polysorbates), polyoxyethylene fatty acid esters (such as Myrj), sorbitan esters (such as Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxomers), polaxamines, methylcellulose, hydroxycellulose, hydroxypropylcellulose, hydroxypropylmethylcellulose, noncrystalline cellulose, poly(vinyl alcohol), poly(vinyl pyrrolidone), and mixtures thereof. Preferably, the non-ionic surfactant is a copolymer of polyoxyethylene and polyoxypropylene, more preferably a block copolymer of propylene glycol and ethylene glycol. Such polymers are sold under the tradename Poloxamer also sometimes referred to as Pluronic. Alternatively, the non-ionic surfactant is poly(vinyl pyrrolidone).

Suitable zwitterionic surfactants are typically selected from alkyl betaines, alkyl amidopropyl betaines, alkyl sulfobetaines, alkyl glycinates, alkyl carboxyglycinates, alkyl amphopropionates, alkyl amidopropyl hydroxysultaines, acyl taurates and acyl glutamates wherein the alkyl and acyl groups have from 8 to 18 carbon atoms such as cocamidopropyl betaine, sodium cocoamphoacetate, cocamidopropyl hydroxysultaine, sodium cocamphopropionate, and mixtures thereof.

The core may optionally further comprise an electrolyte. Examples of typical electrolytes include the sodium salts of acetates, phosphates and citrates, and mixtures thereof.

The core may optionally further comprise a pharmaceutically acceptable acid. Typically, the acid is selected from the group consisting of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, L-ascorbic acid, L-aspartic acid, benzenesulfonic acid, benzoic acid, boric acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dehydro acetic acid, dodecylsulfuric acid, edetic acid, ethane-1,2-disulfonic acid, ethanesulfonic acid, formic acid, fumaric acid, galactaric acid, gentisic acid, D-glucoheptonic acid, D-gluconic acid, D-glucuronic acid, glutamic acid, glutaric acid, glycerophosphoric acid, glycolic acid, hippuric acid, hydrobromic acid, hydrochloric acid, isobutyric acid, isostearic acid, DL-lactic acid, lactobionic acid, lauric acid, maleic acid, L-malic acid, malonic acid, DL-mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, (−)-L-pyroglutamic acid, salicylic acid, sebacic acid, sorbic acid, stearic acid, succinic acid, sulfuric acid, (+)-L-tartaric acid, thiocyanic acid, para-toluenesulfonic acid, undecylenic acid, and mixtures thereof. Preferably, the acid is citric acid.

The core may optionally further comprise a pharmaceutically acceptable small molecule that is fast-dissolving in water. Typically, the small molecule is a compound wherein greater than 75% by weight of the compound, preferably greater than 85% by weight of the compound, more preferably greater than 90% by weight of the compound, even more preferably greater than 95% by weight of the compound, and most preferably greater than 98% by weight of the compound dissolves within a period of 15 minutes when 1 gram of the compound is added to 100 mL of water under stirring at 25° C.

The core may additionally comprise a colorant. The colorants are used in amounts effective to produce a desired colour. Examples of colorants include, but are not limited to, titanium dioxide, colours of natural foods and edible dyes.

The core may additionally comprise a pore-forming agent. Examples of suitable pore-forming agents include, but are not limited to, water-soluble compounds and hydrophilic polymers. Typical water-soluble compounds may include alkali metal salts (such as sodium chloride, sodium bromide, and the like), alkaline earth metals (such as calcium phosphate, calcium nitrate and the like), transition metal salts (such as ferric chloride, ferrous sulfate and the like), polyglycols, ethyl vinyl alcohols, glycerin, pentaerythritol, polyvinyl alcohols, vinylpyrrolidone, N-methyl pyrrolidone, saccharides, hydrolyzed starch, pregelatinized starch, carbohydrates (such as glyceraldehydes, erythrose, ribose, arabinose, xylose, glucose, mannose, galactose, maltose, lactose, sucrose and the like), sugar alcohols (such as mannitol and the like), and mixtures thereof. Typical hydrophilic polymers may include hydroxypropyl cellulose, hydroxypropylmethyl cellulose (HPMC), poly(vinyl pyrrolidone) (PVP), and mixtures thereof.

The core may additionally comprise a plasticizer. A “plasticizer”, as defined herein, is a material that improves the processing of release-controlling agents, which are described below. Examples of suitable plasticizers include, but are not limited to, adipates, azelates, benzoates, citrates, isoebucaes, phthalates, sebacates, stearates, tartrates, polyhydric alcohols, glycols, and the like. Preferred plasticisers include acetylated monoglycerides, butyl phthalyl butyl gylcolate, dibutyl tartrate, diethyl phthalate, dimethyl phthalate, ethyl phthalyl ethyl glycolate, glycerin, ethylene glycol, propylene glycol, triethyl citrate, triacetin, acetylated triacetin, tripropinoin, diacetin, dibutyl phthalate, acetyl monoglyceride, polyethylene glycols, tributyl citrate, castor oil, polyhydric alcohols, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylexyl trimellitate, di-2-ethylexyl adipate, di-2-ethylhexyl sebacate, di-2-ethylhexyl azelate, di-n-butyl sebacate, glyceryl monocaprylate, glycerol tributyrate, glycerol distearate, glyceryl monocaprate, natural, semi-synthetic and synthetic glycerides, monoglyceride, acetylated monoglycerides, fractionated coconut oil, rape oil, olive oil, sesame oil, castor oil, hydrogenated castor oil, acetyltributylcitrate, acetyltriethylcitrate, glycerin sorbitol, diethyl oxalate, diethyl malate, diethyl fumarate, dibutyl succinate, diethyl malonate, dioctyl phthalate, and mixtures thereof.

The core may additionally comprise any suitable preservative. A “preservative”, as defined herein, may refer to: (i) a chelating agent; (ii) an antioxidant; or (iii) an antimicrobial agent.

Typically, the preservative may be any suitable chelating agent. A “chelating agent”, as defined herein, refers to a chemical compound that is a multidentate ligand that is capable of forming two or more separate bonds to a single central atom, typically a metal ion. Examples of suitable chelating agents include, but are not limited to: ethylenediaminetetraacetic acid (EDTA), ethylene glycol-bis(β-aminoethyl ether)-N,N,N′,N′-tetraacetic acid (EGTA), 1,2-bis(ortho-aminophenoxy)ethane-N,N,N′,N′-tetraacetic acid (BAPTA), citric acid, phosphonic acid, glutamic acid, histidine, malate, and derivatives thereof.

Alternatively, the preservative may be any suitable antioxidant. An “antioxidant”, as defined herein, is any compound that inhibits the oxidation of other chemical species. Examples of suitable antioxidants include, but are not limited to: ascorbic acid; citric acid; sodium bisulfite; sodium metabisulfite; and butyl hydroxitoluene.

Alternatively, the preservative may be any suitable antimicrobial agent. An “antimicrobial agent”, as defined herein, is any compound that kills microorganisms or prevents their growth. Examples of suitable antimicrobial agents include, but are not limited to: benzyl alcohol; benzalkonium chloride; benzoic acid; methyl-, ethyl- or propyl-paraben; and quarternary ammonium compounds.

As can be determined from the above, the skilled person will readily appreciate that a single chemical entity could be added to formulations of the present invention to achieve different technical functions. For example, poly(vinyl pyrrolidone) may be added to the formulations to act as a carrier, a binder, a surfactant, or a pore-forming agent, or to act in two or more of these capacities.

Typically, the core comprises granules, crystals or pellets comprising the API, food supplement or vitamin, and optionally one or more pharmaceutically acceptable excipients. Preferably, the core comprises granules, crystals or pellets which are spherical, or substantially spherical, in shape. Alternatively, the core consists of, or consists essentially of, granules which are spherical, or substantially spherical, in shape.

In one embodiment, the core comprises granules of the API, food supplement or vitamin, preferably an API. Optionally, said granules are produced from milled API in a wet or dry granulation process.

In one embodiment, the core comprises crystals of the API, food supplement or vitamin, preferably an API.

In one embodiment, the core comprises pellets comprising the API, food supplement or vitamin. Optionally in this embodiment, said pellet comprises (i) a neutral core comprising one or more excipients, and (ii) an outer layer comprising the API, food supplement or vitamin, preferably an API. Typically, this outer layer surrounds the neutral core. In some embodiments, multiple layers of API, food supplement or vitamin may be present around the core, for example 2, 3, 4, 5, 6 or more layers of API, food supplement or vitamin. In said embodiments, each of the layers may be the same or different to one another. Thus, typically, each of the layers comprises the same components. Alternatively, different layers, preferably adjacent layers, comprise different components, e.g. a different API, food supplement or vitamin. Optionally, the API may be present in the core of the pellet.

### (b) Features of the First Coating Layer of the Granules

The core is preferably surrounded by a first coating layer. The function of the first coating layer is to mask the taste of the core, act as a physical barrier around the core and/or to delay the onset of release of the API, food supplement or vitamin following administration of the formulation. Typically, therefore, the first coating layer comprises a taste-masking agent, a physical barrier coating and/or an enteric coating. Alternatively, the first coating layer consists of a taste-masking agent, a physical barrier coating and/or an enteric coating.

An “enteric coating” is a coating which is resistant to degradation in the acidic environment of the stomach after oral administration of the pharmaceutical formulation, but which dissolves in the more alkaline environment of the intestine (e.g. the small intestine). Therefore, an enteric coating ensures that the contents of the core are released in one or more mammalian intestinal sites chosen from the duodenum, jejunum, ileum, and colon following oral administration of the pharmaceutical formulation. This may be desirable in certain cases, e.g. when the API, food supplement or vitamin decomposes in an acidic environment, or when the API, food supplement or vitamin may cause irration to the stomach of the subject. Enteric coatings are discussed, for example, Loyd, V. Allen, Remington: The Science and Practice of Pharmacy, Twenty-first Ed., (Pharmaceutical Press, 2005; and P. J. Tarcha, Polymers for Controlled Drug Delivery, Chapter 3, CRC Press, 1991, all of which are incorporated herein by reference. Methods for applying enteric coatings to pharmaceutical compositions are well known in the art, and include for example, U.S. Patent Publication No. 2006/0045822, the contents of which are incorporated herein by reference. Examples of suitable enteric coatings include, but are by no means limited to, acrylic acid, methacrylic acid or ethacrylic acid polymers or copolymers, cellulose acetate (and its succinate and phthalate derivatives), hydroxypropyl methyl cellulose phthalate, polyvinyl acetate phthalate, hydroxyethyl ethyl cellulose phthalate, cellulose acetate tetrahydrophtalate, acrylic resin, shellac, cellulose acetate phthalate (CAP; dissolves above pH 6), polyvinyl acetate phthalate (PVAP, disintegrates above pH 5), hydroxypropyl methyl cellulose phthalate (HPMCP, grade HP50 disintegrates above pH 5 and HP50 disintegrates above 5.5), and methylacrylic acid copolymers (e.g. Eudragit L 100 and L12.5 disintegrate between about pH 6 and about pH 7, Eudragit L-30 and L100-55 disintegrate at a pH above 5.5 and Eudragit S100, S12.5 and FS 30D disintegrate at a pH above 7).

A “physical barrier” coating is one which protects the contents of the core (i.e. the API, food supplement or vitamin) from the external environment. In particular, a “physical barrier” coating may impart moisture protection, i.e. prevents moisture from contacting the API, food supplement or vitamin during routine storage of the pharmaceutical formulation. Coating layers which have the function of a physical barrier coating and/or act as a taste-masking agent include hydroxyethylcellulose, hydroxypropylmethylcellulose, sodium carboxymethylcellulose, gelatin, poly(vinyl pyrrolidone), PVA-PEG copolymer (i.e. poly(vinyl alcohol)-poly(ethylene glycol) copolymer), poly(vinyl alcohol), a PVA-PEG/PVA mixture, amino dimethyl methacrylate copolymer, amino diethyl-methacrylate copolymer, sodium alginate, shellac, carboxymethylcellulose, cellulose acetate phthalate, cellulose acetate butyrate, methacrylic acid copolymer, methacrylic acid copolymer (Type A), methacrylic acid copolymer (Type B), methacrylic acid copolymer (Type C), ethyl cellulose, cellulose acetate, poly(ethyl acrylate-co-methyl methacrylate) 2:1, ammonio methacrylate Type A:poly(ethyl acrylate-co-methyl methacrylate-co-trimethylammonioethyl methacrylate chloride) 1:2:0.2, ammonio methacrylate Type B:poly(ethyl acrylate-co-methyl methacrylate-co-trimethylammonioethyl methacrylate chloride) 1:2:0.1, and poly(vinyl acetate). Other suitable examples of taste-masking agents are as defined elsewhere in this disclosure.

Preferably, the first coating layer dissolves at least to some extent in water. Without wishing to be bound by any particular theory, first coating layers which do not dissolve to any appreciable extent in water and which are hydrophobic in nature may be undesirable in use in a straw. That is because the second coating layer of the granules dissolves rapidly in water, exposing the first coating layer to the surrounding aqueous solution. If the first coating layer does not dissolve to any appreciable extent in water and is hydrophobic in nature, the granules may form agglomerates in the straw following the rapid dissolution of the second coating layer. “Agglomerates”, as defined herein, are solid masses that result from the coalescence of two or more granules comprising a core and a first and/or second coating layer(s). It is thought that formation of agglomerates which arise from the coalescence of two or more granules that comprise only a core and a first coating layer (i.e. granules in which the second coating layer has already dissolved rapidly in use in a straw) may result in clogging of the straw, which is undesirable.

Preferably, the first coating layer comprises at least one polymer. Said polymer is preferably selected from gelatins, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polyvinyl alcohol, polycarbophil, polyethylene glycol (PEG), polyethylene oxides, polyoxyalkylene derivatives, polymethacrylates, poly(vinyl pyrrolidone) (PVP), polyvinylalcohol (PVA), polyvinyl acetate (PVAc), PVP-vinylacetate-copolymer (PVP-VA), a vinylpyrrolidone-vinyl acetate copolymer (such as Kollidon® VA 64), sodium carboxymethyl cellulose, sodium alginate, xantham gum, locust bean gum, chitosan, cross-linked high amylase starch, cross-linked polyacrylic acid (carbopol), a polyvinylalcohol—polyethyleneglycol-copolymer, ammonio methacrylate Type A, ammonio methacrylate Type B, and mixtures thereof.

Particularly preferred polymers include polyvinylalcohol-polyethyleneglycolcopolymer and polyvinylalcohol.

Typically, the first coating layer may also contain other coating excipients such as plasticisers, colorants and talc which are well known in the art. The plasticiser may be any of the plasticiser compounds described as such in this specification. Particularly suitable plasticisers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers will typically contain 10-25% by weight of a plasticizer, especially dibutyl phthalate, polyethylene glycol, triethyl citrate and triacetin.

Typically, the first coating layer may comprise two or more discrete sublayers. Thus, the first coating layer may comprise two, three, four, five, six, seven, eight, nine, ten or more discrete sublayers. This may be desirable where two separate coatings on the core are beneficial. For example, the first sublayer (i.e. the sublayer closest to the core) may be an enteric coating to delay the onset of release of the API, food supplement or vitamin, but a second sublayer may be employed as a taste-masking agent, or to prevent agglomeration of the granules in use in a straw if the inner enteric coating (first sublayer) does not dissolve to an appreciable extent in water. In such an embodiment, the second sublayer typically comprises a hydrophilic coating that dissolves to an appreciable extent in water, such as any of the preferable polymers set out above. As an example, if a Eudragit polymer is used as the first sublayer, preferably a second sublayer comprising a hydrophilic polymer is employed.

In some embodiments, if the first coating layer comprises two or more discrete sublayers, the outermost sublayer (i.e. the sublayer furthest from the core) comprises a polymer selected from gelatins, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polyvinyl alcohol, polycarbophil, polyethylene glycol (PEG), polyethylene oxides, polyoxyalkylene derivatives, polymethacrylates, poly(vinyl pyrrolidone) (PVP), polyvinyl alcohol (PVA), polyvinyl acetate (PVAc), PVP-vinylacetate-copolymer (PVP-VA), a vinylpyrrolidone-vinyl acetate copolymer (such as Kollidon® VA 64), sodium carboxymethyl cellulose, sodium alginate, xantham gum, locust bean gum, chitosan, cross-linked high amylase starch, cross-linked polyacrylic acid (carbopol), a polyvinylalcohol-polyethyleneglycol-copolymer, ammonio methacrylate Type A, ammonio methacrylate Type B, and mixtures thereof.

Preferably, a further coating layer is present between the first coating layer and the second coating layer. Typically, this further coating layer comprises a hydrophilic but only partially water-soluble material, i.e. a hydrophilic material that dissolves to an appreciable extent in water but at a slow rate. Typically this further coating layer comprises a hydrophilic but only partially water-soluble polymer. Typically the further coaring layer comprises a sustained-release polymer. Preferably the further coating layer comprises poly(vinyl alcohol), ammonio methacrylate Type A, ammonio methacrylate Type B, or a mixture thereof. More preferably, the further coating layer comprises poly(vinyl alcohol). Most preferably, the further coating layer comprises fully hydrolysed poly(vinyl alcohol) grades. Typically when present, the further coating layer substantially coats, e.g. essentially entirely coats, the first coating layer, and is substantially coated by, e.g. essentially entirely coated by, the second coating layer. The function of this further coating layer is to prevent exposure of the first coating layer to the aqueous surroundings, in order to prevent agglomeration of the particles (which may lead to clogging of the straw) and/or to prevent sticking of the particles to the walls of the straw (which may lead to incomplete delivery of the API). Preferably in this embodiment, the further coating layer comprises a pore-forming agent. This allows channels to form in the further coating layer, which enables water to access and dissolve the components of the first coating layer and/or the core. Preferable pore-forming agents include cross-linked poly(ethylene glycol)-poly(vinyl alcohol) (e.g. Kollicoat IR), hydroxypropylmethyl cellulose (HPMC), hydroxyethylcellulose (HEC), sodium carboxymethylcellulose (NaCMC), poly(ethylene glycol) (PEG), povidones and ionic salts. Preferable ionic salts include halides of Group 1 or Group 2 metals such as NaCl, KCl, CaCl-₂, MgCl₂, l NaBr or KBr, and preferably NaCl. A further advantage of the further coating layer is that it enables the particles to be visible in the straw for a substantial time after water is initially passed through the straw, e.g. at least 30 minutes, preferably at least 45 minutes, more preferably at least 60 minutes, and most preferably at least 90 minutes after water is initially passed through the straw. This is useful in a clinical setting, e.g. a nurse or a caregiver can inspect the straw some time after the patient has taken medication through the straw, to ascertain whether the patient has ingested the full desired dose or whether some of the dose remains in the straw.

Embodiments in which a “further” coating layer as described above is present between the first coating layer and the second coating layer are equivalent to embodiments in which no “further” coating layer is present, but wherein the first coating layer comprises two or more sublayers, and wherein the outermost sublayer of the first coating layer is a hydrophilic but only partially water-soluble material, i.e. wherein the outermost sublayer of the first coating layer is a “further” coating layer as described above. Thus, in some preferable embodiments, the first coating layer comprises two sublayers, the first sublayer (i.e. the sublayer closest to the core) comrising an enteric coating, e.g. an enteric polymer, and/or a taste masking agent, and the second sublayer (i.e. the sublayer furthest from the core) being a layer which prevents agglomeration of the granules in use in a straw if the inner enteric coating and/or taste-masking layer (first sublayer) does not dissolve to an appreciable extent in water, preferably wherein the second sublayer comprises a hydrophilic coating that dissolves to an appreciable extent in water, such as those described above. Alternatively, the first coating layer comprises three sublayers, the first sublayer comprising an enteric coating, the second sublayer comprising a taste-masking agent, and the third sublayer comprising a hydrophilic coating that dissolves to an appreciable extent in water, such as those described above. Alternatively, the first coating layer comprises three sublayers, the first sublayer comprising an a taste-masking agent, the second sublayer comprising an enteric coating, and the third sublayer comprising a hydrophilic coating that dissolves to an appreciable extent in water, such as those described above.

Typically, the first coating layer is a smooth layer when observed using optical or electron microscopy, e.g. scanning electron microscopy. Typically, if present, the further coating layer is also a smooth layer when observed using optical or electron microscopy, e.g.

scanning electron microscopy. A smooth appearance is typical when polymers forming a film on core particle following coating. Typically the smoothnes of the first coating layer is improved by way of a curing step subsequent to the coating.

### (c) Features of the Second Coating Layer of the Granules

The second coating layer typically comprises particles of a sugar, a sugar alcohol or any mixture thereof. Examples of suitable sugars and sugar alcohols include, but are not limited to, saccharides (such as monosaccharides, disaccharides, and polysaccharides), glucose, arabinose, lactose, dextrose, sucrose, fructose, maltose, trehalose, dextrins, galactose, mannitol, erythritol, maltitol, isomaltitol, sorbitol, xylitol, lactitol, and mixtures thereof. Sugars and sugar alcohols are fast-dissolving in water.

It is preferable that the second coating layer does not disintegrate too rapidly in water. Without wishing to be bound by any particular theory, it is thought that if the second coating layer disintegrates too rapidly in water, then during the manufacture of the pharmaceutical formulation, the second coating layer may be applied incompletely to the optionally coated core particles. Further, it is thought that during use of the straw comprising the pharmaceutical formulation, the optional first coating layer would be exposed too quickly to the surrounding environment which may lead to adherence of the coated core particles to the walls of the straw (which would lead to incomplete delivery of the API, food supplement or vitamin). Thus, in a particularly preferred embodiment, the second coating layer comprises maltitol, mannitol, trehalose or a mixture thereof, since these are a slow-dissolving sugar alcohols. Thus, in this embodiment, the second coating layer may comprise at least two substances that are sugars or sugar alcohols, wherein one substance is maltitol, mannitol, trehalose or a mixture thereof. For example, the second coating layer may comprise (i) maltitol, mannitol, trehalose, or a mixture thereof, and (ii) at least one sugar or sugar alcohol selected from saccharides (such as monosaccharides, disaccharides, and polysaccharides), glucose, arabinose, lactose, dextrose, sucrose, fructose, maltose, dextrins, galactose, erythritol, isomaltitol, sorbitol, xylitol, and lactitol. In a preferred embodiment, the second coating layer comprises (i) trehalose, maltiol, or a mixture thereof, and (ii) at least one sugar or sugar alcohol selected from saccharides (such as monosaccharides, disaccharides, and polysaccharides), glucose, arabinose, lactose, dextrose, sucrose, fructose, maltose, dextrins, galactose, erythritol, isomaltitol, sorbitol, xylitol, and lactitol. In a more preferable embodiment, the second coating layer comprises (i) trehalose, maltitol, or a mixture thereof, and (ii) erythritol. In a particularly preferred embodiment, the second coating layer comprises erythritol and maltitol. Preferably the ratio of (i) to (ii) is from 1:20 to 1:10, for example about 1:15. Typically (i) is added as a granualting liquid. Typically (ii) is added as in dry form, such as a milled form.

Typically, the second coating layer is a rough layer when observed using optical or electron microscopy, e.g. scanning electron microscopy. Under a microscope, discrete particles of sugar and sugar alcohols can be observed around the smooth first coating layer of the granules (when said first coating layer is present), or around the smooth surface of the core. Without wishing to be bound by any particular theory, it is believed that the rough surface of this layer contributes to the desirable properties of the formulation that are observed in use in a straw. In particular, the rough surface, combined with the fast-dissolving properties of sugar and sugar alcohol particles, is believed to lead to a lower vacuum pressure required to ingest a formulation comprising these particles from a straw, and a smaller volume of liquid required to pass through the straw to administer the formulation.

Typically, the second coating layer comprises from 1 to 100 wt % sugar, sugar alcohol or any mixture thereof, preferably from 10 to 99.9 wt %, more preferably from 25 to 99 wt %, yet more preferably from 50 to 98 wt % and most preferably from 75 to 95 wt %.

Typically, the diameter of the discrete particles of a sugar, sugar alcohol, or any mixture thereof is from 5 to 1000 times smaller than the diameter of the core, preferably from 10 to 500 times smaller, more preferably from 15 to 200 times smaller, and more preferably from 20 to 100 times smaller. Typically, the volume of the discrete particles of a sugar, sugar alcohol, or any mixture thereof is from 5 to 1000 times smaller than the volume of the core, preferably from 10 to 500 times smaller, more preferably from 15 to 200 times smaller, and more preferably from 20 to 100 times smaller.

Typically, the diameter of the granule (i.e. the granule comprising the core, first coating layer and second coating layer) is up to five times greater than the diameter of the core, preferably up to three times greater, more preferably up to two times greater, still more preferably up to 1.5 times greater, and most preferably up to 1.2 times greater. Typcially, therefore, the d₅₀ of granules in the pharmaceutical formulation is up to five times greater than the d₅₀ of the core, preferably up to three times greater, more preferably up to two times greater, still more preferably up to 1.5 times greater, and most preferably up to 1.2 times greater.

Alternatively, the second coating layer may comprise particles of one or more pharmaceutically acceptable fast-dissolving excipients other than a sugar or a sugar alcohol. Preferably, said fast-dissolving excipient is a hydrophilic compound, for example a compound which has a solubility of greater than 50 g/100 g of water at 25° C. at 1 atm pressure, preferably greater than 75 g/100 g water, more preferably greater than 100 g/100 g water, yet more preferably greater than 150 g/100 g water, and most preferably greater than 200 g/100 g water. An example of a fast-dissolving excipient other than a sugar or a sugar alcohol is poly(ethylene glycol). Another example is citric acid.

The second coating layer may additionally comprise one or more pharmaceutically acceptable excipients selected from carriers, diluents, binders, glidants and disintegrants, such as those defined herein. Alternatively, however, the second coating layer may consist of, or consist essentially of, particles of one or more fast-dissolving excipients, preferably particles of a sugar, a sugar alcohol or any mixture thereof.

The second coating may optionally further comprise a taste-masking agent, flavouring agent or sweetener. Examples of suitable taste-masking agents or flavouring agents comprise, e.g., cinnamon, wintergreen, eucalyptus, spearmint, peppermint, menthol, anise, fruit flavors (such as apple, pear, peach, strawberry, cherry, apricot, orange, watermelon, banana and the like), bean-derived flavors (such as coffee, cocoa and the like), or mixtures thereof. Examples of suitable sweeteners include, but are not limited to, D-tagatose, dried invert sugar, corn syrup solids, sodium saccharin, aspartame, sugarless sweeteners including glycerol, hydrogenated starch hydrolysates, and the like, or mixtures thereof. If a taste-masking agent, flavouring agent or sweetener is present, it is typically present in an amount from 0.001 to 10 wt % relative to the total weight of the second coating layer, preferably from 0.001 to 1 wt %, and most preferably about 0.1 wt %.

The second coating may optionally further comprise a binder, such as a polymer.

Suitable polymers for this purpose include any of the polymers that may be present in the first coating layer, and preferably the polymer is selected from gelatins, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polyvinyl alcohol, polycarbophil, polyethylene glycol (PEG), polyethylene oxides, polyoxyalkylene derivatives, polymethacrylates, poly(vinyl pyrrolidone) (PVP), polyvinylalcohol (PVA), polyvinyl acetate (PVAc), PVP-vinylacetate-copolymer (PVP-VA), a vinylpyrrolidone-vinyl acetate copolymer (such as Kollidon® VA 64), sodium carboxymethyl cellulose, sodium alginate, xantham gum, locust bean gum, chitosan, cross-linked high amylase starch, cross-linked polyacrylic acid (carbopol), a polyvinylalcohol—polyethyleneglycol-copolymer and mixtures thereof. If the second coating comprises a binder, the binder is present in an amount from 0.001 to 10 wt % relative to the total weight of the second coating later, preferably from 0.01 to 5 wt %, more preferably from 0.1 to 3 wt %, still more preferably from 0.5 to 2 wt %, and most preferably about 1 wt %.

In one embodiment, the second coating layer does not comprise any additional excipients. Thus, in this embodiment the second coating layer preferably consists of particles of a sugar, a sugar alcohol, or any mixture thereof.

### Manufacture of the Pharmaceutical Formulations

The granules of the present invention are typically prepared by a method comprising the following steps:
(a) providing granules, crystals or pellets of an active pharmaceutical ingredient (API), food supplement or vitamin;
(b) optionally, applying a first coating layer to the said granules, crystals or pellets; and
(c) applying a second coating layer using a wet coating method or a liquid-assisted coating method in a high shear mixer, wherein the second coating layer comprises particles of a sugar, sugar alcohol, or any mixture thereof.

Preferably, step (b) is carried out.

In particular, step (c) ensures that the granules have the characteristic rough surface, whereby discrete particles of a sugar, sugar alcohol, or any mixture thereof can be detected on the surface of the first coating layer (or core, if the first coating layer is absent) by microscopy, which is believed to impart beneficial properties on the granules in use in a straw. This is surprising, because typically a high shear mixer is not used for coating of particles, but rather for granulation. For the avoidance of doubt, a “high shear mixer” may also be referred to as a “high shear granulator”. Thus, what would have been expected from combining particles of two different types in a high shear mixer (e.g. sugar particles and coated API particles) would be the formation of composite granules comprising both particle types dispersed throughout the granule. However, in this instance, it is surprisingly found that the high shear mixer/granulator can effectively coat the coated core granules, crystals or pellets comprising an API, food supplement or vitamin with particles of a sugar, sugar alcohol, or any mixture thereof, to form granules with a characteristic rough surface. Without wishing to be bound by any particular theory, it is believed that this wet coating or liquid-assisted coating in a high shear mixer is possible because the particles of the sugar, sugar alcohol, or any mixture thereof are significantly smaller than the core granules, crystals or pellets. Preferably therefore, the particles of sugar, sugar alcohol or mixture thereof are milled. Preferably, the particles of sugar, sugar alcohol or mixture thereof have a diameter of less than 250 μm, more preferably less than 200 μm, and most preferably less than 150 μm. Without wishing to be bound by any particular theory, it is believed that the smaller milled hydrophilic excipients primarily adhere to the coated particles by wet capillary forces, rather than being agglomerated to each other. Higher levels of agglomeration of the excipients is observed as the water content during the granulation step is increased.

Step (c) preferably comprises a wet coating method. A wet coating method is a method of coating a particulate formulation using a liquid binder (e.g. water) to act as the vehicle that brings the coating (here, the particles of sugar and/or sugar alcohol) into contact with the surface of the substrate (here, the optionally coated core granules, crystals or pellets). The resultant coated granules are typically dried following the coating step. The wet coating method in step (c) preferably comprises spraying or dropping of a liquid binding solution onto the product of step (b) (i.e. a core coated with the first coating layer), wherein the liquid binding solution is an aqueous solution comprising a sugar, sugar alcohol, or any mixture thereof, and optionally one or more pharmaceutically acceptable excipients selected from carriers, diluents, binders, glidants and disintegrants, such as those defined herein. Alternatively, the wet coating method in step (c) may comprise spraying or dropping of a liquid binding solution onto the product of step (a), if no first coating layer is added to the formulation.

Step (c) alternatively comprises a liquid-assisted coating method. A liquid-assisted coating method is a form of dry coating of a particulate formulation, wherein typically no drying step is required after coating. Instead, the liquid phase vehicle that is used to bring the coating into contact with the surface of the substrate is used in a low amount, and will typically remain in the final granulate product, typically acting e.g. as an adhesive agent.

Further details of typical liquid-assisted coating methods are described in Sauer et al., Int J Pharmaceutics, 2013, 457, 488-502, the contents of which are herein incorporated by reference in their entirety. When step (c) comprises a liquid-assisted coating method, typically the liquid vehicle employed is selected from polyhydric alcohols (such as propylene glycol, glycerol, polyethylene glycols, and the like), acetate esters (such as triacetin, triethyl citrate, acetyl triethyl citrate, and the like), phthalate esters (such as diethyl phthalate, and the like), glycerides (such as acetylated monoglycerides, and the like), oils (such as castor oil, mineral oil, and the like), and mixtures thereof. Thus, the method of the present invention advantageously enables the formation of granules which have surprisingly beneficial properties when used in a drinking straw. In particular, a lower vacuum pressure is required to ingest a formulation comprising these granules from a straw, and a smaller volume of liquid is required to pass through the straw to administer the solution. The formulation is considered to be “in use in the straw” when a pressure gradient is applied along the length of the straw containing the formulation, e.g. by a person sucking on one end of the straw when the opposing end is inserted into a liquid.

The method of the present invention is exemplified in FIG. 18. It differs from conventional granulation techniques (as depicted in FIG. 18(a)), in which particles of API are granulated and then coated. Rather, in the present method, particles of API are optionally coated (e.g. with the first coating layer) prior to the granulation step (i.e. the step in which the second coating layer is applied). This process is depicted in FIG. 18(b). FIG. 18(b) illustrates a specific embodiment of the present invention in which a pellet having a neutral core layered with API is provided in step (a), which embodiment is discussed further below. In some instances, discrete coated particles according to the present invention form agglomerates through adherence of the second coating layers of adjacent particles. This particular embodiment is shown in FIG. 18(b).

The granules, crystals or pellets provided in step (a) of the method may be obtained by any method well-known to the skilled artisan. Optionally, any of the pharmaceutically acceptable excipients described herein are added in this preparation step. For example, granules may be formed by grinding or milling, crystals may be obtained by cooling crystalisation or evaporative crystallisation, and pellets may be formed by a sedimentation-centirfugation process. Granules, crystals or pellets having a particular size distribution may optionally then be obtained using a dry sieving method. Thus, the granules, crystals or pellets may be subjected to a dry sieving process using mechanical agitation in order to obtain the required particle size distribution. A typical dry sieving method is described in detail in the US Pharmacopeia at http://www.pharmacopeia.cn/v29240/usp29nf24s0_c786.html, the contents of which are herein incorporated by reference in their entirety. The ISO nominal aperture sieve sizes used for preparation of the pharmaceutical formulations of the present invention may be selected from 50 μm, 53 μm, 60 μm, 63 μm, 74 μm, 75 μm, 80 μm, 90 μm, 95 μm, 100 μm, 106 μm, 112 μm, 125 μm, 140 μm, 150 μm, 160 μm, 180 μm, 200 μm, 212 μm, 250 μm, 300 μm, 315 μm, 335 μm, 355 μm, 400 μm, 425 μm, 450 μm, 475 μm, 500 μm, 560 μm, 600 μm, 630 μm, 700 μm, 710 μm, 800 μm, 850 μm, 900 μm, 1000 μm, 1180 μm, 1200 μm, 1400 μm, 1500 μm, 1700 μm and 2000 μm.

Typically, when a sieve of a particular size is used, at least 90% of the particles by number which pass through the sieve have a diameter of less than the sieve pore size, preferably at least 95% of the particles by number, and most preferably at least 98% of the particles by number. High sieve loading can however decrease the efficiency of the sieving process.

Particle size may be measured by any suitable technique known to the skilled person, such as the methods described in WO 2009/135646 or Marks and Sciarra, Journal of Pharmaceutical Sciences, 1968, 57(3), 497-504. Such techniques may include mechanical sieving methods, laser light diffraction and/or scanning electron microscopy.

In one embodiment, in the case of milled API (e.g. if the API has low water solubility), an increase in particle size is achieved by a first agglomeration step using either a dry or wet granulation technique, carried out in the presence of one or more excipients, to provide the granules of step (a). Typically, the size of the granules thus produced is from 250 to 710 μm.

In one embodiment, the granules, crystals or pellets are pellets. In this embodiment, the pellet typically comprises a neutral core and an outer layer comprising the API, food supplement or vitamin, preferably an API. Typically, this outer layer surrounds the neutral core. In some embodiments, multiple layers of API, food supplement or vitamin may be present around the core, for example 2, 3, 4, 5, 6 or more layers of API, food supplement or vitamin. In said embodiments, each of the layers may be the same or different to one another. Thus, typically, each of the layers comprises the same components. Alternatively, different layers, preferably adjacent layers, comprise different components, e.g. a different API, food supplement or vitamin. Typically, the diameter of said pellets is from 200 μm to 600 μm.

The first coating step (b) can be carried out by any conventional coating method known to the skilled person. Conventional coating techniques such as fluid bed or Wurster coaters, or spray or pan coating are employed to apply coatings. Preferably, the first coating layer is applied using fluid bed coating or high shear melt coating. The coating thickness must be sufficient to ensure that the core remains intact until the desired site of delivery in the body is reached. Typically, the core is coated with a single coating layer, selected from a taste-masking agent, physical barrier coating and/or an enteric coating. Alternatively, however, the coating step is repeated once, twice, three times or more, to produce cores having multiple sublayers of the first coating layer. Each sublayer may be the same or different. Preferably, two adjacent sublayers of the first coating are different to one another.

Most preferably, each subcoating layer is different to one another.

In one embodiment, the first coating step (b) comprises two distinct substeps:
(b1) applying a first coating layer to the said granules, crystals or pellets; and
(b2) applying a further coating layer that is hydrophilic but partially water-soluble onto the first coating layer.

Thus, in one embodiment, the granules of the present invention are prepared by a method comprising the following steps:
(a) providing granules, crystals or pellets of an active pharmaceutical ingredient (API), food supplement or vitamin;
(b1) applying a first coating layer to the said granules, crystals or pellets;
(b2) applying a further coating layer that is hydrophilic but partially water-soluble onto the first coating layer; and
(c) applying a second coating layer onto the further coating layer using a wet coating method or a liquid-assisted coating method in a high shear mixer, wherein the second coating layer comprises particles of a sugar, sugar alcohol, or any mixture thereof.

In this embodiment, preferred features of steps (a) and (c) are as described above. Preferred features of (b1) are as described above for step (b). In this embodiment, preferably the water-insoluble layer is hydrophilic. The further coating layer (b2) can be applied using any conventional technique, e.g. the techniques mentioned in respect of the first coating step (b1) above.

Following the preparation of granules via steps (a)-(c) outlined above, granules having a particular size distribution may typically be obtained using a dry sieving method, such as those described above.

Thus, the present invention provides a pharmaceutical formulation suitable for use in a straw suitable for oral administration of said pharmaceutical formulation, wherein the pharmaceutical formulation is solid and comprises granules, and the granules comprise:
(a) a core comprising an active pharmaceutical ingredient (API), food supplement or vitamin;
(b) optionally, a first coating layer surrounding the core; and
(c) a second coating layer surrounding the first coating layer and/or the core, the second coating layer comprising particles of a sugar, a sugar alcohol, or any mixture thereof;

wherein the second coating layer is obtainable using a wet coating method in a high shear mixer.

Preferably, the wet coating method comprises spraying or dropping of a liquid binding solution onto a larger core coated particles mixed with smaller excipient particles with the first coating layer in high shear mixer. Preferably, the first coating layer is present. Preferably, the first coating layer is obtainable by fluid bed coating or high shear melt coating.

### Uses of the Pharmaceutical Formulations in Treatment

In general, formulations of the present invention are administered to a human patient so as to deliver to the patient a therapeutically effective amount of the active pharmaceutical ingredient (API). The formulations are administered orally.

Thus, the present invention further relates to a pharmaceutical formulation as described herein for use in the treatment of a condition in a subject in need thereof, wherein the pharmaceutical formulation comprises an API, and said treatment comprises oral administration of the pharmaceutical formulation using a straw, wherein the straw contains the pharmaceutical formulation.

In another aspect, the present invention also provides a method of treating a condition in a subject in need thereof, said method comprising oral administration of a pharmaceutical formulation as described herein using a straw, wherein the pharmaceutical formulation comprises an API, and the straw contains the pharmaceutical formulation.

In another aspect, the present invention also provides the use of a pharmaceutical formulation as described herein for the manufacture of a medicament for the treatment of a condition in a subject in need thereof, wherein the pharmaceutical formulation comprises an API, and said treatment comprises oral administration of the pharmaceutical formulation using a straw, wherein the straw contains the pharmaceutical formulation.

Typically, in the aspects described above, the oral administration is effected by the steps of: (i) insertion of one end of said straw in an aqueous solvent and insertion of the other end of said straw in the oral cavity of the subject; and (ii) the application of suction by the subject to the end of the straw situated in the oral cavity.

Typically, oral administration of at least 90% of the formulation is achieved when a volume of 100 mL or less of aqueous solvent, preferably 50 mL or less, more preferably 40 mL or less, even more preferably 30 mL or less, and most preferably 20 mL or less, is passed through the straw. Preferably, oral administration of at least 95% of the formulation is achieved when a volume of 100 mL or less of aqueous solvent, preferably 50 mL or less, more preferably 40 mL or less, even more preferably 30 mL or less, and most preferably 20 mL or less, is passed through the straw. More preferably, oral administration of at least 98% of the formulation is achieved when a volume of 100 mL or less of aqueous solvent, preferably 50 mL or less, more preferably 40 mL or less, even more preferably 30 mL or less, and most preferably 20 mL or less, is passed through the straw. Most preferably, oral administration of at least 99% of the formulation is achieved when a volume of 100 mL or less of aqueous solvent, preferably 50 mL or less, more preferably 40 mL or less, even more preferably 30 mL or less, and most preferably 20 mL or less, is passed through the straw.

### Devices Comprising the Formulation

The present invention further relates to a device suitable for oral administration of a pharmaceutical formulation as described above, wherein the device contains a pharmaceutical formulation as described herein. Typically, the device is configured to allow the pharmaceutical formulation to be flushed from the device by a volume of aqueous solvent which is 50 mL or less, preferably 30 mL or less, more preferably 20 mL or less, during oral administration and to thereby deliver the aqueous solution in which the pharmaceutical formulation is flushed into the oral cavity of a subject. Typically, the device is configured to allow suction applied by the subject to (a) bring the aqueous solvent into contact with the pharmaceutical formulation, and (b) deliver the pharmaceutical formulation into the oral cavity.

Suitable devices may include straw and lidded beakers, e.g. child drinking beakers or so-called “sippy” cups. Preferably, the device is a straw. Thus, the present invention further relates to a straw suitable for oral administration of a pharmaceutical formulation as described above, wherein the straw contains the pharmaceutical formulation as described above. Any straw suitable for this purpose falls within the scope of the present invention. Such a straw containing said pharmaceutical formulation is referred to herein as a “pre-filled” straw.

Typically, the device containing the pharmaceutical formulation of the present invention is configured such that when aqueous solvent is passed through the device (e.g. a straw) for 60 seconds, the d₅₀ value of the resulting formulation is typically 100 μm or more, preferably 150 μm or more, and more preferably 200 μm or more, e.g. from 200 μm to 500 p.m, preferably from 200 μm to 400 μm, and more preferably from 200 to 300 μm. This feature advantageously enables dose-uptake recognition.

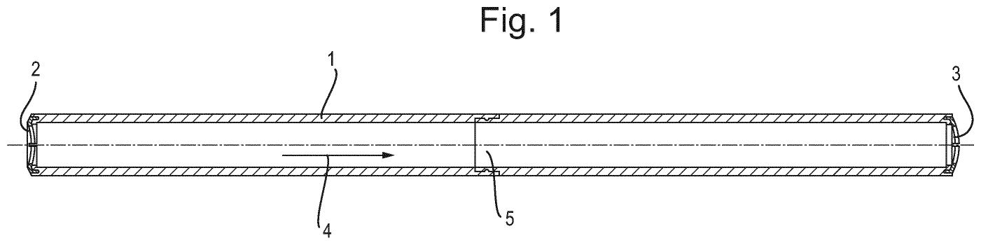

A particularly preferred type of pre-filled straw suitable for oral administration of the pharmaceutical formulation is described in more detail below, with reference to FIGS. 1 to 8.

The parts of a preferred pre-filled straw according to the invention are depicted in FIG. 1 and are a main straw body 1 in tubular form, which may be round or oblong shape and two cross slit valves 2, 3. The main straw body includes at least two straw segments, each having a straw body and a respective valve at one end. Valve 2 is positioned on the liquid inlet and valve 3 on the outlet of the straw. The valves 2, 3 are positioned in the way to allow only one-way flow through the body straw, as presented by arrow 4 in FIG. 1. The valves 2, 3 are initially in a closed position, but when suction in the direction of the arrow 4 is applied, both of them are opened and allow the liquid to enter the straw. When suction is stopped, both valves 2, 3 return to a closed position.

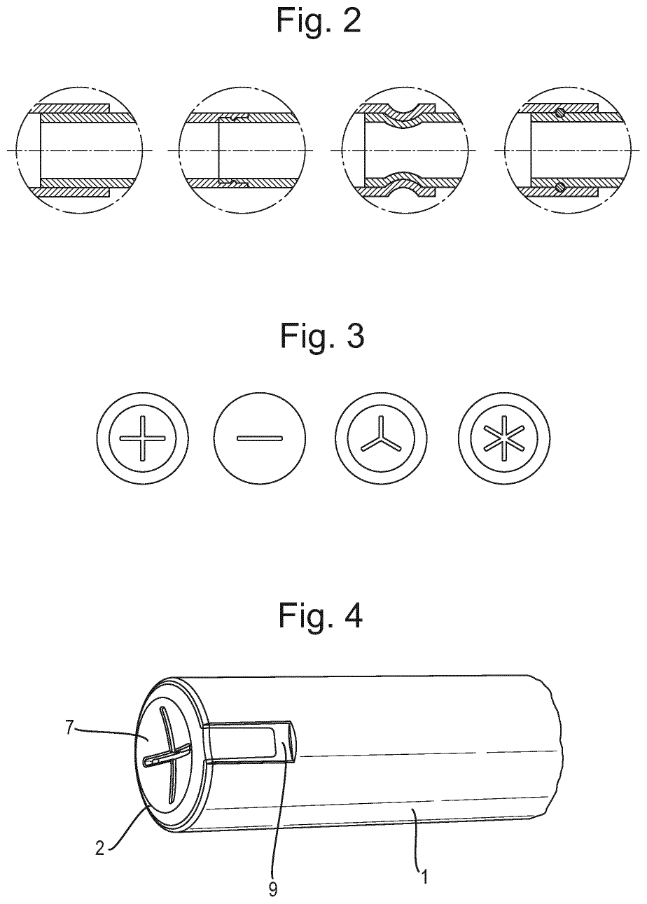

Straws consisting of two or even more than two segments are envisaged by the present invention. In the arrangement shown in FIG. 2, the straw has two straw segments, each with a respective valve 2, 3 at one end of the segment. The two straw segments are directly coupled to each other. In other arrangements, the two straw segments having a valve at one end may be coupled to each other indirectly, for example by way of one or more additional straw segments.

The segments are coupled together with a coupling 5. Such couplings may be formed from two elements formed, each formed at an end of a respective straw segment. The elements on each segment are configured such that the element on one straw segment can engage with the element on another straw segment to couple the straw segments together. In such a way the straw segments can be coupled together after the straw segments have been formed. This is in contrast with the valves, which may be integrally formed as part of the straw segment.

Several types of coupling are depicted in FIG. 2 that may be used to couple the straw segments. Straw segments may, for example, be attached one to another by a friction fit connection, i.e. one part is slightly narrower than the other, a snap-fit connection, a press-fit connection, a weld, a section of adhesive or another suitable coupling. A snap-fit connection may be an annular snap joint, or capsule like closing system, in which one straw segment has a U-shaped annular groove around the cross-section of the straw segment that, when the straw segments are coupled, receives corresponding protrusion on the other straw segment.

The thickness of the wall of the straw segment may be increased in the region of the coupling. This may increase the strength, reduce the risk of cracking and/or make it easier to mould the elements of the coupling.

Once the coupling has been completed, it may be configured to be not possible to detach the straw segments without damage. Alternatively the coupling may be configured to be detachable, permitting a user to open the straw to pour and/or wash out the contents.

In an arrangement the coupling between the straw segments may provide a gas tight, or hermetic, seal. This may ensure that, when a user sucks on one end of the straw, pressure is sufficiently reduced within the straw to draw liquid into the other end of the straw.

Depending on the coupling used, an O-ring may be provided in order to ensure a good seal between the straw segments.

As depicted in FIG. 1, first and second straw segments coupled together to form a pre-filled straw may have the same length. Alternatively, the two straw segments may have different lengths. For example, it may be desirable for one segment to be longer than the other. This may be beneficial if a pharmaceutical formulation according to the present invention as described above is to be placed in only one of the straw segments prior to coupling the two straw segments together because this may permit a greater amount of formulation to be placed within the pre-filled straw for a given total length of pre-filled straw. However, longer straw segments are more difficult to form due to cooling of the mould during straw formation. Accordingly, the length of a straw segment containing the formulation during preparation of the pre-filled straw need to be smaller than the length of the overall pre-filled straw.

In general, the choice of total length of pre-filled straw may be a compromise. It may be selected to be long enough to be convenient for a user to ensure that it reaches liquid in the bottom of a cup. However, the longer the straw, the harder the user must suck to draw up liquid into their mouth. Furthermore, longer straws cost more to manufacture and take up more space in storage and transit.

In FIG. 3 possible arrangements of slits in the cross-slit valves are presented. The slit valves per se are known. Valves are moulded and may be made of elastomer material. Slits can be cut in a shape of a cross, of a line, of a three-pointed star, of a six-pointed star or any other appropriate form. The cross-slit valves can differ in shapes, as presented in the figure with the concave round shape, duck-bill shape and similar.

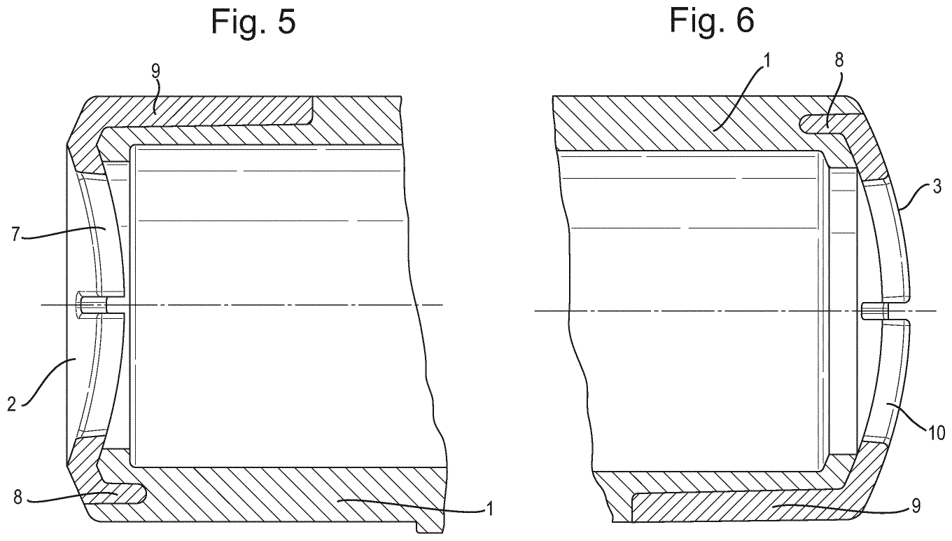

An inlet valve 2 according to the present disclosure is presented in FIGS. 4, 5. As shown it may be integrally formed, for example connected and melded, or co-moulded, to the lower end of the straw body 1. Straw body 1 may be made of thermoplastic and the valve 2 with a membrane 7 that may be made of an elastomer, such as a thermoplastic elastomer. Other materials that can be co-moulded to the straw body. The membrane 7 of the inlet valve is bent towards the inside of the straw body 1, i.e. the membrane 7 is concave.

In an arrangement, the material used to form the straw body may be transparent or translucent. In use, this may enable the user to confirm that all of the formulation within the straw has been consumed.

In an arrangement, one or both of the inlet valve 2 and the outlet valve 3 may be formed in a distinctive colour. If both the inlet valve 2 and the outlet valve 3 are coloured they may have different distinctive colours. Arrangements with one or more valve having a distinctive colour may assist in indicating to users the correct orientation of the pre-filled straw in use. For example, user instructions may include a pictogram that uses the one or more coloured valves to clearly indicate the end to be inserted in the mouth and the end to be inserted in a liquid.

Alternatively or additionally, markings may be provided on the straw body to indicate correct orientation of the straw for use and/or the direction of liquid flow in use. Such markings may be applied to the straw body by any appropriate means, including printing on the straw body, application of stickers and the inclusion of surface patterns within the mould design.

The edge of the straw body 1 may be shaped to enable larger surface of the connection between the straw body 1 and the inlet valve 2. The said shape is preferably a recess, such as an indent or groove 8, formed inside the edge of the wall of the straw body 1. To enable the injection of the thermoplastic into the valve-shaped mould, a tongue-shaped groove 9 may be formed on the surface side on the end of the straw body 1. Said groove 9 enables that the injected thermoplastic flows from the injection unit to fill in the valve 2 mould.

An outlet valve 3 is presented in FIG. 6 and may have generally the same structure as the inlet valve 2. The outlet valve 3 is bent towards the outside of the straw body 6, i.e. the membrane 10 is convex.

The inlet and outlet valves 2, 3 with the membranes 7, 10 may be injection-moulded directly onto respective straw segments of the main straw body 1. As discussed above, the straw body may have a groove 8. During the injection moulding process, when the elastomer is injected onto the straw body 1, a junction between both materials, i.e. the thermoplastic of the straw body 1 and the elastomer of valves 2, 3, is formed by the adhesive molecular forces. The provision of the groove may increase the area of this contact.

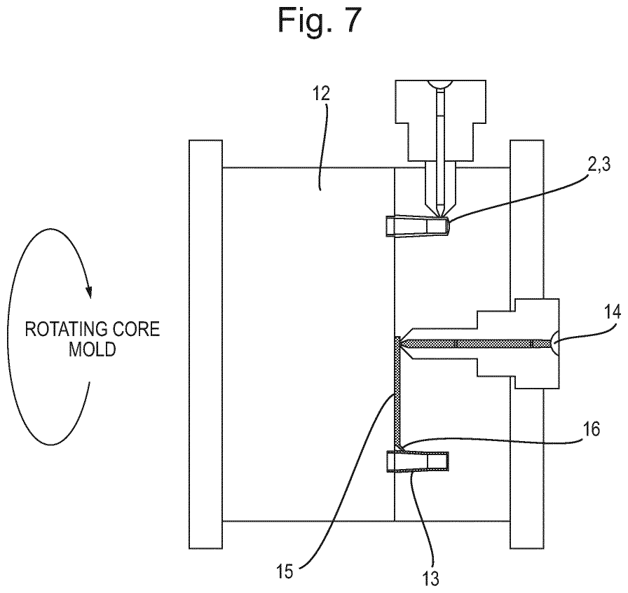

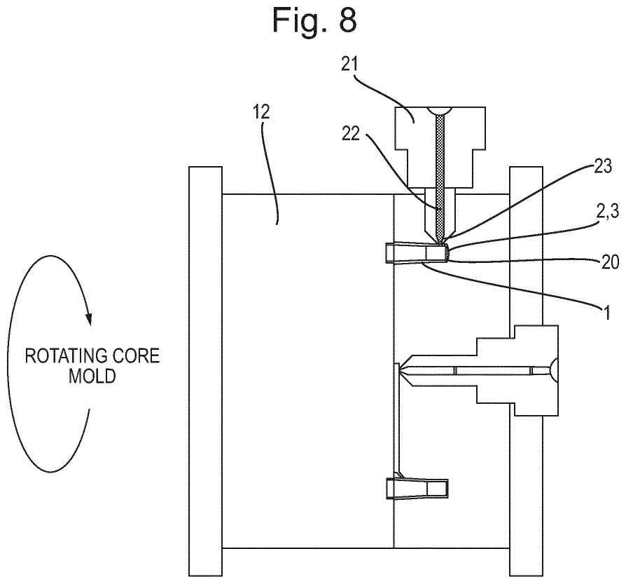

FIGS. 7 and 8 depict a two component, i.e. thermoplastic and elastomer, injection moulding process. The process is performed by injecting the first component, preferably polymer, into the mould 12. In the first step the first component is injected thorough the injection nozzle 14 into the channel 15. Then the first component flows through the gate 16 to the appropriate cavity 13 in the shape of straw body segment 1. The flow enters the mould 12 through the gate 16 into the groove 9. In this cavity 13 the straw body 1 is formed. After this process is completed, the mould 12 changes the configuration in order to initiate the second step of the process.

Before the polymer is cooled-off or is hardened, the tooling configuration is changed, i.e. the mould 12 rotates and changes the configuration in order to initiate the second step of the process. Then follows the injection of the second material, preferably elastomer into the cavity and thus cross slit valve is moulded onto the straw body. In this way the valves and straw are attached by molecular adhesion. This approach allows the production cycle time to be shortened.

In the second step as presented in FIG. 8 the already formed straw body 1 comes in contact with the second cavity 20. The second cavity 20 is in the shape of the valve 2, 3. The second component, preferably elastomer, is injected form the injection nozzle 21 through the second channel 22 and enters the cavity 20 through the gate 23. After cooling the finished piece is ejected from the mould 12.

In an arrangement, the straw bodies may be formed with a tapered shape. In particular the straw bodies may be arranged such that the cross-sectional area of the opening within the straw is smaller at the end having the cross-slit valve than its other end, namely the end that may be coupled to another straw segment. The latter end may be generally open, in contrast to the end that is closed by the cross-slit valve. Such an arrangement may facilitate the removal of the straw segment from the mould once formation of the straw segment is completed. In an arrangement one or both of the straw segments having cross-slit valves integrally formed at one end may have a frusto-conical shape.

With the said moulding process several straw sections are produced which later are to be coupled together to form a straw, as described above.

In particular, the pre-filled straw may be prepared by placing a formulation according to the present invention to be orally administered within a first straw segment that has an integrally formed cross-slit valve such as discussed above. Next, a second straw segment that has an integrally formed cross-slit valve may be coupled to the first straw segment. Such a process may be easier than previously known processes for preparing a pre-filled straw because it may preclude the need to attach a valve to a straw that contains a formulation according to the present invention to be orally administered. This may reduce spillage of the formulation during the process and/or reduce costs.

In an arrangement, when the formulation is being placed in the first straw segment, the integrally formed valve of the first segment may prevent loss of the formulation from the first straw segment. For example, during the process of placing the formulation within the first straw segment, it may be held with the integrally formed valve below the other end such that, to the extent that the formulation flows, it flows towards the integrally formed valve, which prevents the formulation leaving the straw segment. Once the second straw segment has been coupled to the first straw segment, the formulation may prevented from leaving the straw in either direction (when the straw is not in use) by the integrally formed valves at either end of the pre-filled straw.

It should be appreciated that other arrangements for filling the straw may be possible. For example, the formulation could be placed in two straw segments with integrally formed valves before the two straw segments are coupled. This may require steps to prevent the formulation falling out of one or both straw segments during coupling.

In any case, it should be appreciated that, although in preparation of the pre-filled straw the pharmaceutical formulation according to the invention and as described herein may be placed initially in one straw segment, once the pre-filled straw has been prepared, the pharmaceutical formulation may partially or completely transfer to another straw segment before use.

In an arrangement, after the formulation has been placed in the straw and the straw segments coupled together, packaging may be added. The packaging may be an enclosure that completely surrounds one or more pre-filled straws and/or may cover one or both of the integrally formed cross-slit valves. Such packaging may prevent accidental leakage of the formulation from the pre-filled straw before use. The packaging may be child-proof packaging. The packaging may also include instructions on how to use a straw according to the present invention.

According to the arrangements disclosed above in this preferred embodiment, a very good prevention against the loss of the straw content is obtained, since both of the cross-slit valves are closed in the time of non-use. The loss of the content during suction is also prevented, as the outlet valve inhibits counter pressure applied into the straw, and inlet valve prevents the loss of the liquid from the straw.

### EXAMPLES

The following are Examples that illustrate the present invention. However, these Examples are in no way intended to limit the scope of the invention.

### Comparative Example 1: Coating of Paracetamol Crystals

Paracetamol crystals ranging in size from 250 to 710 μm obtained by sieving were subsequently coated with polymer to achieve a taste masking effect. Kollicoat® IR (polyvinyl alcohol/polyethylene glycol graft copolymer, BASF Germany), Kollicoat® Protect (combination of water-soluble Kollicoat® IR and polyvinyl alcohol, BASF Germany), Eudragit® E PO (copolymer based on dimethylaminoethyl methacrylate, butyl methacrylate, and methyl methacrylate, Evonik Germany), Pharmacoat 603 (hydroxypropylmethyl cellulose (HPMC), Shin-Etsu Chemical Co, Japan) and Gelatin (Merck, Germany) were used as taste masking coating excipients. The Wurster method was used for coating and experiments were performed in Glatt GPGC1 (Binzen, Germany) apparatus. 500 g of powder was added to the Wurster chamber, and the coating suspension was added flowing through 1.2 mm bottom nozzle. The amount of added coating suspension was around 25% by weight of the dry product (except for the case of Eudragit® E PO, where 13% of dry product mass coating was applied). The process parameters are listed in Table 1, and coating suspension compositions in Table 2.

Eudragit® E PO suspension was prepared by first dissolving SDS powder in the required amount of water using an Ultra-turrax stirrer (IKA, Germany). After 10 min of stirring, an Eudragit® E PO powder was added portionwise, and the stirring process was maintained for further 60 min. Talc was subsequently added directly into the polymer colloid suspension while stirring, and the suspension was homogenized for several more minutes. The preparation of the other polymer suspensions consisted of two steps. The total required amount of water was divided into halves. Polymer was added to the first half of the water in a glass beaker under stirring using a magnetic stirrer. The beaker was then covered with aluminum foil preventing water evaporation, and stirring was maintained overnight. The next day, talc was added to the other half of the water and homogenized using Ultra-turrax. The talc suspension was mixed with the polymer dispersion to obtain the coating suspension.

Before the coating step, all prepared polymer suspensions were sieved through a 0.5 mm sieve to prevent nozzle clogging during the process. Gelatin, Kollicoat® Protect, and Kollicoat® IR suspensions were heated to 60° C. during the coating process to achieve an appropriate spraying liquid viscosity, while the Eudragit® E PO and HPMC suspensions were sprayed at room temperature. After polymer coating was completed, the coated crystals were dried to maximal 1% moisture content (determined with the loss on drying (LOD) test). The dried product was later sieved to obtain a particle size range between 250 and 800 μm.

The loss on drying test (LOD) was performed at 80° C. for 20 min using a infrared moisture analyzer (Buchi B-302, Switzerland), where 3-5 g of sample was placed on an aluminum plate. The final result was expressed as the weight loss percentage.

TABLE 1
Process parameters for coating of paracetamol crystals in Wurster chamber
(Glatt GPGC1)

| | | Suspension | | | | |
|---|---|---|---|---|---|---|
| | Air | feeding | Inlet air | Outlet air | Product | Atomizing |
| Polymer | flow | rate | temperature | temperature | temperature | air |
| suspension | (m³/h) | (g/min) | (° C.) | (° C.) | (° C.) | (bar) |
| Kollicoat ® | 81.0-85.0 | 5-7 | 50-55 | 40-42 | 40-42 | 1.7-2.0 |
| Protect | | | | | | |
| Kollicoat ® | 81.0-85.0 | 5-6 | 53-55 | 40-42 | 41-42 | 1.7-2.0 |
| IR | | | | | | |
| HPMC | 74.2-85.0 | 7-9 | 53-55 | 39-41 | 40-41 | 2.0-2.2 |
| Gelatin | 63.4-81.0 | 7 | 40-60 | 35-47 | 35-37 | 1.5-1.7 |
| Eudragit ® | 63.3-67.0 | 6-8 | 40-43 | 26-28 | 26-28 | 1.2-1.5 |
| EPO | | | | | | |

TABLE 2
Compositions of aqueous polymer coating suspensions

| | | Solid |
|---|---|---|
| Suspension | | concentration |
| type | Components | (wt %) |
| Kollicoat ® | Kollicoat Protect | 12.0 |
| Protect | Talc | 6.0 |
| Kollicoat ® IR | Kollicoat IR | 7.0 |
| | Talc | 4.4 |
| HPMC | Pharmacoat 603 | 6.0 |
| | PEG 6000 | 1.2 |
| | Talc | 4.5 |
| Gelatin | Gelatin | 7.0 |
| | Talc | 5.6 |
| Eudragit ® | Eudragit ® E PO | 8.0 |
| EPO | SDS | 0.8 |
| | Talc | 4.0 |

### Scanning Electron Microscopy

The pure (uncoated) paracetamol crystals and each of the coated crystals were analyzed using a scanning electron microscope (Supra 35VP variable pressure field emission SEM, Carl Zeiss, Oberkochen, Germany). The crystals were deposited directly on double-sided carbon tape or cut with stainless steel scalpel for coss section observation (Oxon,

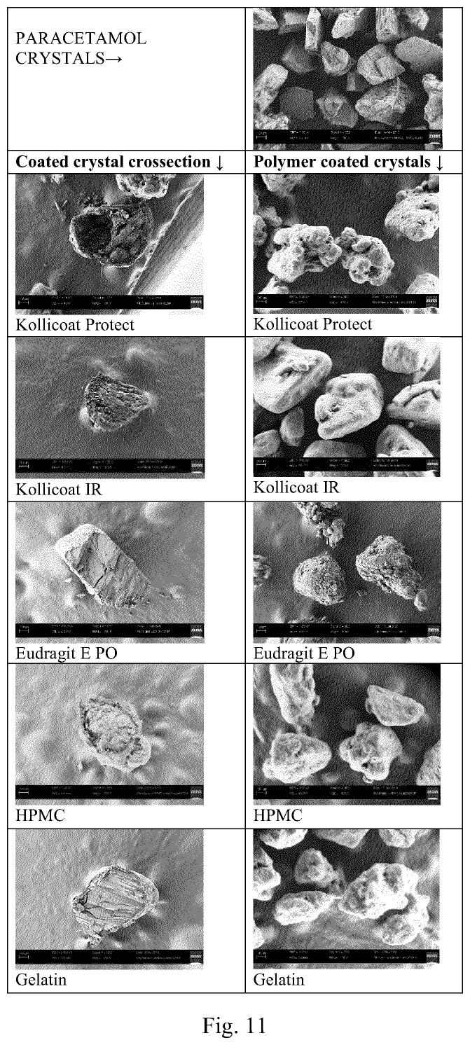

Oxford Instruments, Abingdon, UK). No conductive coating was applied to the samples. High vacuum mode was used at a beam acceleration voltage between 0.8 and 1.2 kV using secondary electron signals and working distance between 3.5 and 5.0 mm. Micrographs were recorded with a magnification of 150 and are shown in FIG. 11.

### Particle Size Analysis

The particle size distribution of powder samples (crystals, coated crystals) was determined using a laser diffraction analyzer (Malvern Mastersizer 3000, Worcestershire, United Kingdom) connected to Aero S dry powder dispersion, as this technique enables measurement in the range between 100 nm and 3500 μm. Approximately 2-3 g of sample was put into the dosing device, air pressure was set to 1.2 bar for coated crystals and granulates (for micronized paracetamol the dispersing air pressure was set to 3.0 bar). Measurement was performed automatically when obscuration was in a range between 0.5 and 5%. Background measurements were performed for 10 seconds and the stabilization time delay before initialization (time before measurements after the desired obscuration was achieved) was set to 3 seconds and measurement duration to 5 seconds respectively. The nonspherical measurement mode approximation was used to determine the particle size distribution for paracetamol crystals and coated crystals. A refraction index and absorption rate for paracetamol were set to 1.62 and 0.01 respectively. The distribution results were expressed as three values: d10, where 10% of the particle population lies below the measured value; d50, the median value; and d90, where 90 percent of the population lies below the measured value. These results are shown in Table 3.

### Sipping Pressures, Volumes and Straw Emptying Times

To measure the performance of the formulations in a straw, preferred drinking straws as shown in FIG. 1 and described in detail above, were employed. The preferred straws comprise a first straw segment, which contains the pharmaceutical formulation and has an integrally formed cross-slit valve at one end, and a second straw segment, which has an integrally formed cross-slit valve at one end, wherein the ends of the first and second straw segments that do not have integrally formed cross-slit valves are coupled to one another.

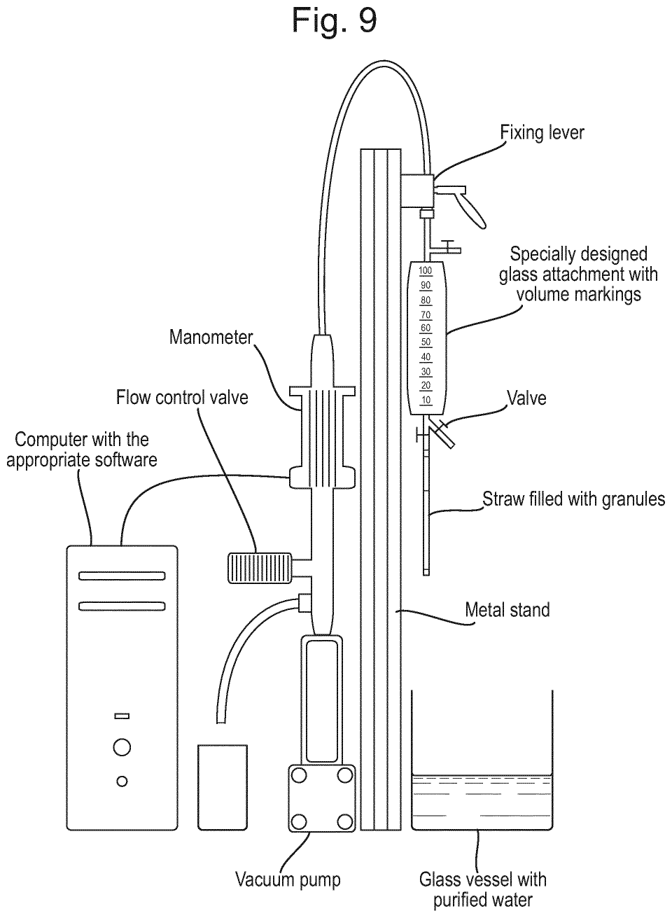

The straw was filled with the formulation being tested, and then attached to a measurement device comprising a pump, pressure sensor, valve and collection trap, as illustrated in FIG. 9. The straw was attached to the collection trap in vertical direction, so that liquid is pumped upwards. The lower end of the straw was placed into a solution containing 100 mL of water. The whole system is linked with a computer containing software to record and manipulate data. The flow was pre-set at a prescribed uniform rate (e.g. 5 mL/s±5%) by placing a graduated pipette in place of the straw. During the experiment, the liquid (i.e. water) was pumped through the straw into the collection trap. The collection trap is graduated, so that it allows the volume pumped through the straw to be measured. The liquid is pumped upwards as the under-pressure is raised in the system by the pump. The under-pressure is continually measured by the sensor for the duration of the experiment and recorded in the software.

The following data points were measured during the course of the experiment:
The change in absolute pressure just prior to liquid passing through the top cross-slit valve in the straw (p₁);
The maximum change of absolute pressure in the straw during the experiment (p₂);
The change in absolute pressure when the straw has been entirely emptied of the formulation (p₃);
The flush volume (V), i.e. the total volume of liquid required to fully carry the formulation out of the upper end of the straw; and
The emptying time (t_{at p2}), which is the time between the start of the experiment and the point at which the maximum change of absolute pressure in the straw is obtained.

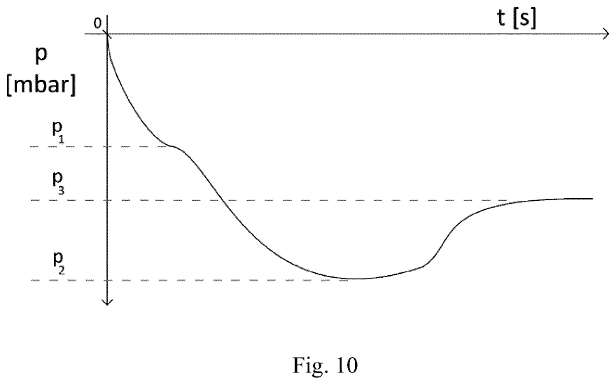

A graphical representation of the change in pressure during the course of the experiment is shown in FIG. 10, which also illustrates the points of the experiment at which p₁, p₂ and p₃ were measured. At the beginning of the experiment, the pressure decreases and at the pressure value of p₁, water reaches the top valve of the straw. After that, the pressure continues to fall and at p₂ the vacuum in the straw reaches its maximum value. Subsequently, as the straw is emptied of the formulation, the vacuum rapidly decreases.

TABLE 3
Particle size results obtained with laser diffraction, maximal sipping pressure
along with flush volume values and emptying time measured for samples containing
250 mg of paracetamol.

| | d₁₀ | d₅₀ | d₉₀ | p₂ | Volume | Emptying time, |
|---|---|---|---|---|---|---|
| Sample | (μm) | (μm) | (μm) | (mbar) | (mL) | t_{at p2} (s) |
| Paracetamol—milled, | 5 | 32 | 152 | −100 | 80 | 17 |
| uncoated | | | | | | |
| Paracetamol—large | 208 | 458 | 772 | −95 | 50 | 11 |
| crystals, uncoated | | | | | | |
| Kollicoat ® IR coated | 426 | 626 | 910 | −118 | 30 | 8 |
| crystals | | | | | | |
| Kollicoat ® Protect | 366 | 572 | 871 | −130 | 60 | 12 |
| coated crystals | | | | | | |
| HPMC coated crystals | 415 | 606 | 870 | −137 | 80 | 22 |
| Gelatin coated crystals | 419 | 628 | 914 | −146 | 50 | 10 |
| Eudragit ® E PO | 406 | 615 | 916 | −141 | >80 | * |

*Straw was not emptied

Example 2: Further Coating of Polymer-Coated Paracetamol Crystals with Sugar or Sugar Alcohol in High Shear Granulator

The polymer-coated crystals described in Comparative Example 1 were wet coated in ProCepT 4M8-TriX (Zelzate, Belgium) high-shear (HS) mixer. Paracetamol-coated crystals (equivalent to 25% of final paracetamol content) having a diameter from 250-800 μm were transferred into a 1 L glass vessel along with milled erythritol and trehalose (mass ratio 1:1). The blend was then stirred in a HS mixer for 15 s at 400 rpm impeller speed. After that, 30% trehalose binding solution was sprayed (0.8 mm nozzle) with a flow rate of 2 g/min and an atomizing air flow rate of 7.0 L/min. During liquid addition, the impeller and chopper speed were set to 400 rpm and 2000 rpm respectively. The grinding time, after complete granulation liquid addition was set to 15 s. Wet granulate was then sieved through a 2 mm sieve with plastic card and dried on trays at 40° C. to obtain granules having less than 1% moisture content by weight (determined with the loss on drying (LOD) test). Dried product was sieved once again through a 2 mm sieve, where granules smaller than 500 μm in diameter were discharged. Prepared granules contained 25% of paracetamol by weight.

Scanning Electron Microscopy

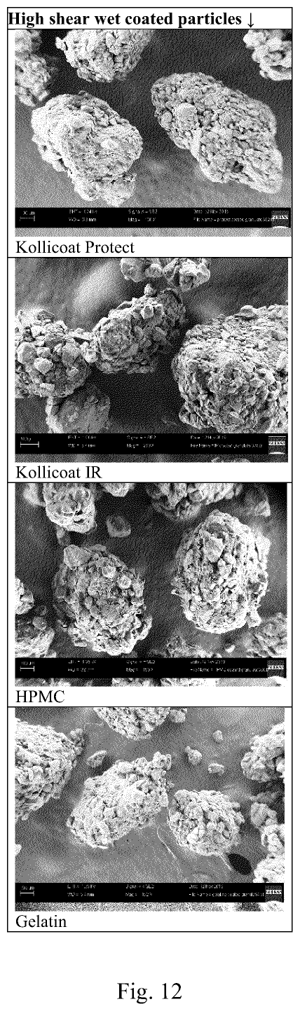

Wet coated granules comprising a core containing paracetamol crystals were analyzed using a scanning electron microscope (Supra 35VP variable pressure field emission SEM, Carl Zeiss, Oberkochen, Germany). The particles were deposited directly on double-sided carbon tape or cut with stainless steel scalpel for cross-section observation (Oxon, Oxford Instruments, Abingdon, UK). No conductive coating was applied to the samples. High vacuum mode was used at a beam acceleration voltage between 0.8 and 1.2 kV using secondary electron signals and working distance between 3.5 and 5.0 mm. Micrographs were recorded with a magnification of 150 and are shown in FIG. 12.

Particle Size Analysis

The particle size distribution of the wet-coated particles was determined as set out above for Comparative Example 1. The results are shown in Table 4.

Sipping Pressures, Volumes and Straw Emptying Times

The performance of the formulations within a straw was tested as set out above for Comparative Example 1. The results are shown in Table 4.

TABLE 4
Particle size results obtained with laser diffraction,
maximal sipping pressure along with flush
volume values and emptying
time measured for samples containing
250 mg of paracetamol.

| | | | | | | Emptying |
|---|---|---|---|---|---|---|
| | d₁₀ | d₅₀ | d₉₀ | p₂ | Volume | time, |
| Sample | (μm) | (μm) | (μm) | (mbar) | (mL) | t_{at p2} (s) |
| Kollicoat ® IR coated | 441 | 720 | 1120 | −112 | 20 | 6 |
| crystals further coated | | | | | | |
| with erythritol | | | | | | |
| Kollicoat ® Protect | 476 | 813 | 1130 | −98 | 10/15 | 5 |
| coated crystals further | | | | | | |
| coated with erythritol | | | | | | |
| HPMC coated crystals | 465 | 744 | 1170 | −124 | 20/30 | 7 |
| further coated with | | | | | | |
| erythritol | | | | | | |
| Gelatin coated crystals | 481 | 715 | 1040 | −117 | 20 | 6 |
| further coated with | | | | | | |
| erythritol | | | | | | |
| Eudragit ® E PO | 488 | 829 | 1390 | −138 | >80 | * |
| coated crystals | | | | | | |
| further coated with | | | | | | |
| erythritol | | | | | | |

* Straw was not emptied

### Conclusions

As seen from Table 4, erithrytol trehalose coated granules are emptied much more easily (i.e. p2 is less negative) and faster (i.e. t_{at p2} is lower) in a lower volume of water from a drinking straw than granules which comprise only a core paracetamol crystal and a polymer coating layer (see Table 3 for comparative data). These dual-coated granules gave acceptable emptying pressure values less negative than −130 mbar and lower emptying volumes than 20 mL. These parameters are desirable characteristics of formulations intended for administration to a patient.

The only exception is granules in which the polymer layer comprises Eudragit® E PO, which could not be emptied from the straw either with or without the additional erithrytol trehalose coating. Eudragit® E PO is the only tested polymer that is from the entero-soluble cationic methacrylate copolymers group. It possesses pH-dependent solubility, and does not dissolve above pH 5. It is hypothesised that during the sipping test, trehalose and erythritol dissolve rapidly in water, exposing the pure polymer surface. Given the lack of solubility of Eudragit® E PO in water at pH 7, it is thought that the polymer-coated granules interact more strongly with each other than with water, resulting in formation of agglomerates that cause clogging of the straw.

The best in-straw behaviour resulting in an easy-sipping formulation was found for erithytol trehalose coated particles obtained in high shear mixer. Granules having Kollicoat® Protect as the first (inner) coating layer even resulted in measured sipping pressures above −100 mbar. Flush volumes in this case were extremely low, with the whole straw content being emptied in less than 20 mL of water. The shorter empying times of trehalose erythritol coated granules is a further indicator that formulations comprising these granules are effective for easy consumption of an active ingredient.

### Example 3: Influence of Amount of Liquid Binder Added on Properties of Polymer-Coated Crystals that are Further Coated in a High Shear Mixer

Two batches of paracetamol crystals coated with polymer Kollicoat® Protect (as described in Comparative Example 1) were wet coated in ProCepT 4M8-TriX (Zelzate, Belgium) high-shear (HS) mixer with erythritol. 53.0 g Kollicoat® Protect coated paracetamol crystals having a diameter from 250-500 μm were transferred into a 1 L glass vessel along with 90.0 g milled erythritol. The blend was then stirred in a HS mixer for 15 s at 400 rpm impeller speed.

### Batch A: Smaller Amount Of Liquid Binder Added

During granulation 14.0 g of 30% maltitol solution was added (containing 9.8 g of water, 0.8 mm nozzle) with a flow rate of 2 g/min and an atomizing air flow rate of 7.0 L/min. During liquid addition, the impeller and chopper speed were set to 400 rpm and 2000 rpm respectively. Wet product was subsequently sieved through a 2 mm sieve with plastic card and dried on trays at 40° C. to obtain granules having less than 1% moisture content by weight (determined with the loss on drying (LOD) test). Dried product was sieved once again through a 2 mm sieve, where particles smaller than 500 μm were discharged. The final composition of the granules is set out in Table 5.

TABLE 5
Composition of granules batch A.

| Component | % weight |
|---|---|
| Kollicoat ® Protect coated | 36.0 |
| crystals (250-500 μm) | |
| (paracetamol content 68.9%) | |
| Milled erythritol | 61.1 |
| Maltitol | 2.9 |
| Total | 100.0 |

### Batch B: Larger Amount of Liquid Binder Added

During granulation 22.0 g of 30% maltitol solution was added (containing 15.4 g of water, 0.8 mm nozzle) with a flow rate of 2 g/min and an atomizing air flow rate of 7.0 L/min. During liquid addition, the impeller and chopper speed were set to 400 rpm and 2000 rpm respectively. Wet product was subsequently sieved through a 2 mm sieve with plastic card and dried on trays at 40° C. to obtain granules having less than 1% moisture content by weight (determined with the loss on drying (LOD) test). Dried product was sieved once again through a 2 mm sieve, where particles smaller than 500 μm were discharged. The final composition of the granules is set out in Table 6.

TABLE 6
Composition of granules batch B.

| Component | % weight |
|---|---|
| Kollicoat ® Protect coated | 35.4 |
| particles (250-500 μm) | |
| (paracetamol content 68.9%) | |
| Milled erythritol | 60.2 |
| Maltitol | 4.4 |
| Total | 100.0 |

### Scanning Electron and Optical Microscopy

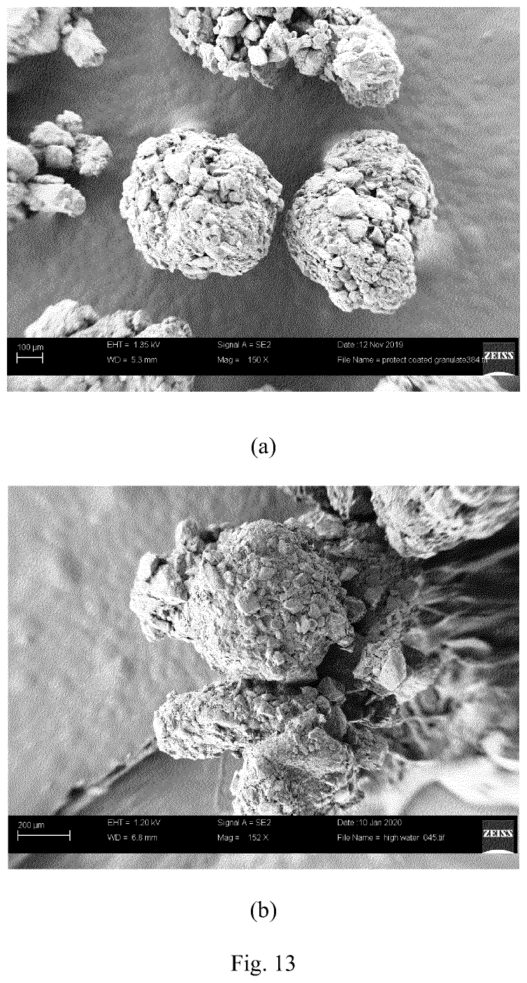

The wet coated granules (batches A and B) were analyzed using a scanning electron microscope following the same protocol as for Example 2 above. Micrographs were recorded with a magnification of 150 and are shown in FIGS. 13a (Batch A) and 13b (Batch B).

Optical microscope images of (a) uncoated paracetamol crystals, (b) Kollicoat® Protect coated crystals and (c) the final granulates with an outer coating of erythritol and maltiol (Batch A) are shown in FIG. 14.

### Particle Size Analysis

The particle size distribution of the wet-coated particles was determined as set out above for Comparative Example 1. Results are shown in Table 7. It can be seen that addition of more binding liquid during the wet coating step results in the formation of larger granules. It is believed that this is due to the agglomeration of some of the discrete erythritol-coated granules.

TABLE 7
Particle size distribution of Kollicoat ® Protect
coated paracetamol crystals,
erythritol particles used for coating and
final granules obtained with high shear mixer.

| Formulation | d₁₀ | d₅₀ | d₉₀ |
|---|---|---|---|
| Kollicoat ® Protect | 305 μm | 435 μm | 615 μm |
| coated crystals | | | |
| Erythritol | 7 μm | 46 μm | 108 μm |
| Batch A | 321 μm | 558 μm | 931 μm |
| Batch B | 571 μm | 990 μm | 1860 μm |

### Conclusions

Addition of both a smaller and greater amount of liquid binding solution of maltitol during the wet-coating step in the high shear mixer causes formation of granules where the granules have a rough morphology due to small sugar or sugar alcohol particles that cover the surface of the polymer-coated crystals (see FIG. 13). When more liquid binding solution is used (i.e. more water is added), agglomerates composed of discrete erythritol-coated granules start to form. Primary granules with an outer coating made from erythritol can however still be observed.

### Comparative Example 4: Water Wettability of Polymers

The water contact angle on compressed powders of the different polymers used as the first coating layer, trehalose and erythritol was measured with a Kruss sessile drop contact angle meter DSA100 (Kr\ddot{υ}ss, Germany) at room temperature. 200 mg of powder was compressed with a Specac hydraulic press (Kent, England) with a 10 s dwell time at a pressure of 4×10⁸ Pa. A circular punch and dye assembly (12 mm diameter) with polished stainless steel round punches were used for compression. At least six repetitions on three different plates of the same polymer or other two excipients were performed to ensure the reproducibility of results. Measurements were performed with 3 μL water drop and video recording (software DSA 1.90.0.14 Kr\ddot{υ}ss, Germany) that enabled the determination of contact angles at time zero and 30 seconds later. Contact angles at these two-time points together with visual observation of surface swelling were taken as relevant parameters for polymers differentiation. In the same manner, trehalose and erythritol were also analyzed. The results are shown in Table 8.

TABLE 8
Sessile drop water contact angles (θ) and observed swelling
(— = not measurable, * = observed, ** = fast swelling,
*** = very fast swelling) during contact
angle measurement for polymer,
trehalose and erythritol flat surface plates.

| | θ at time 0 s/° | θ at time 30 s/° | | Observed |
|---|---|---|---|---|
| Test substrate | (st. deviation) | (st. deviation) | Δ θ/° | sweling |
| Water soluble | | | | |
| polymer | | | | |
| Kollicoat IR | 33.1 (2.1) | 15.0 (1.1) | 18.1 | *** |
| Kollicoat Protect | 77.6 (2.4) | 60.6 (2.1) | 17.0 | * |
| HPMC | 64.8 (0.8) | 56.2 (2.0) | 8.6 | * |
| Gelatin | 71.9 (2.2) | 59.8 (2.1) | 12.1 | ** |
| Entero-soluble | | | | |
| cationic | | | | |
| methacrylate | | | | |
| copolymer | | | | |
| Eudragit E PO | 56.4 (1.9) | 53.7 (1.6) | 2.7 | — |
| Fast dissolving | | | | |
| excipient | | | | |
| Trehalose | 13.4 (1.9) | 8.6 (2.0) | 4.8 | — |
| Erytrhitol | 0 | 0 | 0 | — |

It is believed that ease of flow for the coated paracetamol crystals during sipping depends predominantly on polymer chemical structure. Further flow improvment of granulated coated crystals can be attibuted to very fast dissolution of erythritol and trehalose in the water and less dense packing of particles during sipping. The true density of particles is according to our opinion less important factor as for different particle samples should not change much.

The balance between contact angle, decrease in contact angle during the first 30 seconds, and polymer swelling ability during the short time that is available for dose application through a straw are believed to be parameters that explain differences in particle behaviour that is observed during the sipping tests of Comparative Example 1 and Example 2. For example, Eudragit E PO has a relatively high water contact angle, a small contact angle decrease in the first 30 seconds and negligible swelling during this time interval. Granules coated with Eudragit E PO also showed the most negative p₂ value in the sipping test, and could not be completely emptied from the straw. In contrast, Kollicoat IR is the most wettable polymer with water, shows the largest decrease of contact angle in the first 30 seconds, and swells to the highest extent. This finding correlates well with the best flow performance of Kollicoat IR coated paracetamol crystals (lowest siping pressure, lowest flash volume) in Comparative Example 1 and Example 2. Further, the improvements in p₂ value, sipping volume and straw emptying times demonstrated when an outer coating of erythritol/trehalose mixture is applying to the granules (Example 2) may also be partly attributed to good wettability and fast dissolution of these two excipients.

### Comparative Example 5: Dissolution Testing of Coated Paracetamol Crystals

The optimum dissolution characteristics for an orally administered formulation are that at the neutral pH in the oral cavity, the API particles are not released particularly quickly, as they have an undersirable taste. The period in which the formulation remains in the oral cavity tends to be short, but may be up to 2 minutes. Once the formulation reaches the stomach, a rapid dissolution in the acidic stomach environment is desirable to prevent a delay in the therapeutic benefit of the paracetamol. For different active agents, though, it is conceivable that a slower rate of dissolution in acid may be beneficial, e.g. for delayed-release formulations where the active agent is preferably absorbed in the duodenum, jejunum or ileum.

### Dissolution of Coated Paracetamol Crystals in Water

A non-compendial test was performed in order to simulate the taste masking efficinecy of polymer coat during drug sipping. Pure paracetamol crystals were used as reference material. 100 mL of purified water was transferred in an Erlenmeyer flask on a magnetic stirrer at room temperature. The test formulation containing 250 mg of paracetamol was suspended in the Erlenmeyer flask and aliquotes of 5.0 mL were withdrawn at intervals of 0.5, 1.0, 1.5, 2.0, 2.5, 3.0 and 5.0 minutes (±15% tolerance limit for time) and replaced with an equal volume of purified water (freshwater, maintained at the same temperature) at each sampling point. Samples were immediately filtered using 0.45 μm RC syringe filters.

Samples were appropriately diluted and analyzed by HPLC. The amount of drug release was calculated from a standard calibration curve, which was prepared on the day of the experiment.

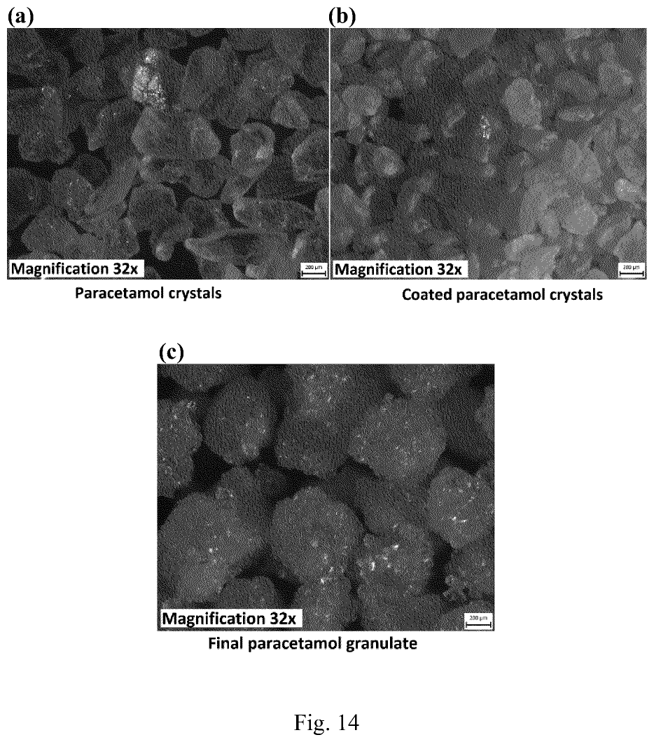

Results are shown in FIG. 15. It can be observed that all formulations efficiently mask the paracetamol taste for first two minutes as the released amount of API is does not exceed 16%. Uncoated paracetamol as expected dissoves much faster than from coated particles.

### Dissolution of Coated and Granulated Paracetamol in Acid Media

For dissolution studies in acid media, the compendial USP paddle method was performed using a Vankel Varian Vk700 dissolution apparatus (Agilent Technologies, USA). Dissolution profiles were obtained from reference (oral suspension of Calpol) and test products tested at 50 rpm in 900 mL of two different dissolution media (prepared in accordance with European Pharmacopoeia): hydrochloric acid media at pH 1.2 and phosphate buffer solution at pH 4.5 (at 37° C.±0.5° C.). Test samples containing 250 mg of paracetamol (single dose) were weighed for analysis. While using an autosampler assembled with a 10 μm flow filters, aliquotes of 5.0 mL were withdrawn at intervals of 2, 5, 10, 15, 20, 30, 45 and 60 minutes.

Samples were appropriately diluted and analyzed by HPLC. The amount of drug release was calculated from a standard calibration curve, which was prepared on the day of the experiment.

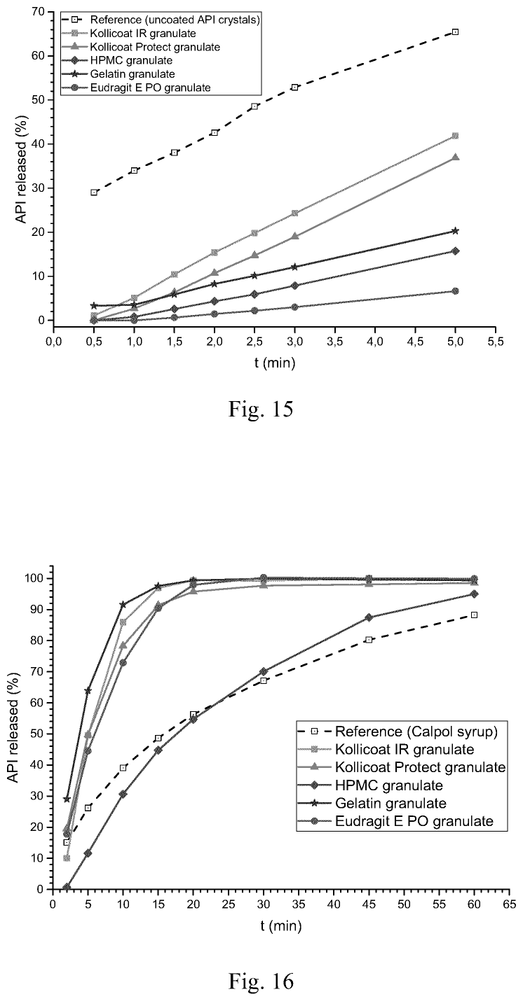

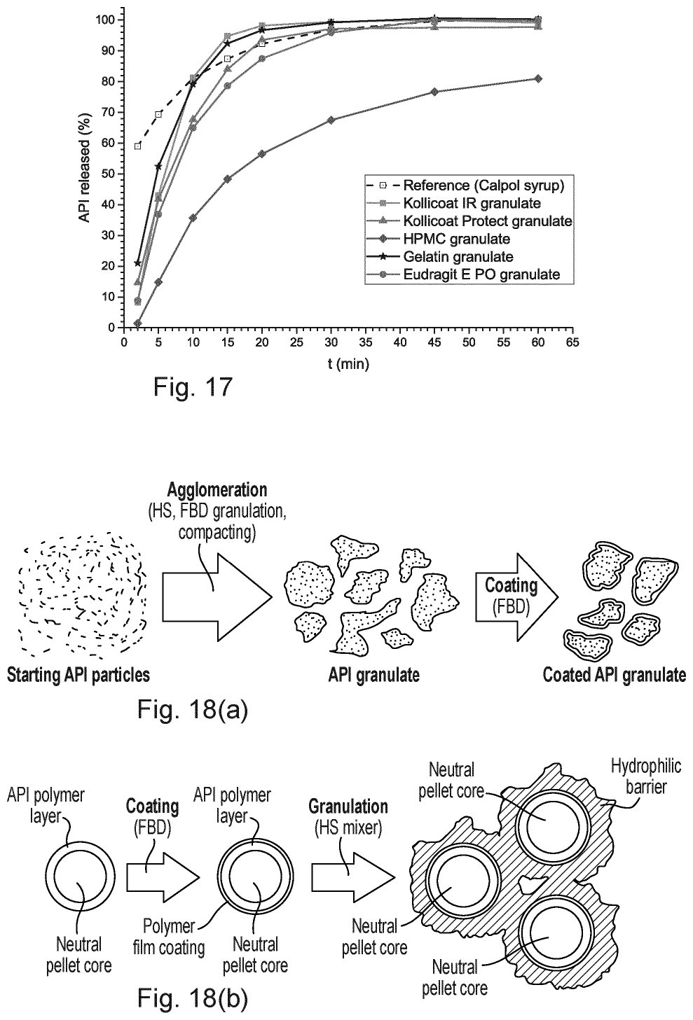

Results are shown in FIGS. 16 and 17. At pH 1.2, the coated crystals release paracetamol much faster when compared with the reference product, Calpol. Four samples release paracetamol almost completely after 20 min. Paracetamol release is slower at pH 4.5, as anticipated. The relative dissolution rates of each granule type correlate to a certain extent with the swelling kinetics that were observed during wettability measurements (see Example 4). For instance, Kollicoat® IR- and gelatin-coated granules are the two fastest-dissolving granules in acid media, and these are also the polymers for which greatest swelling was observed in the wettability testing.

### Example 6: Dissolution Testing of Sucrose-Coated Granules

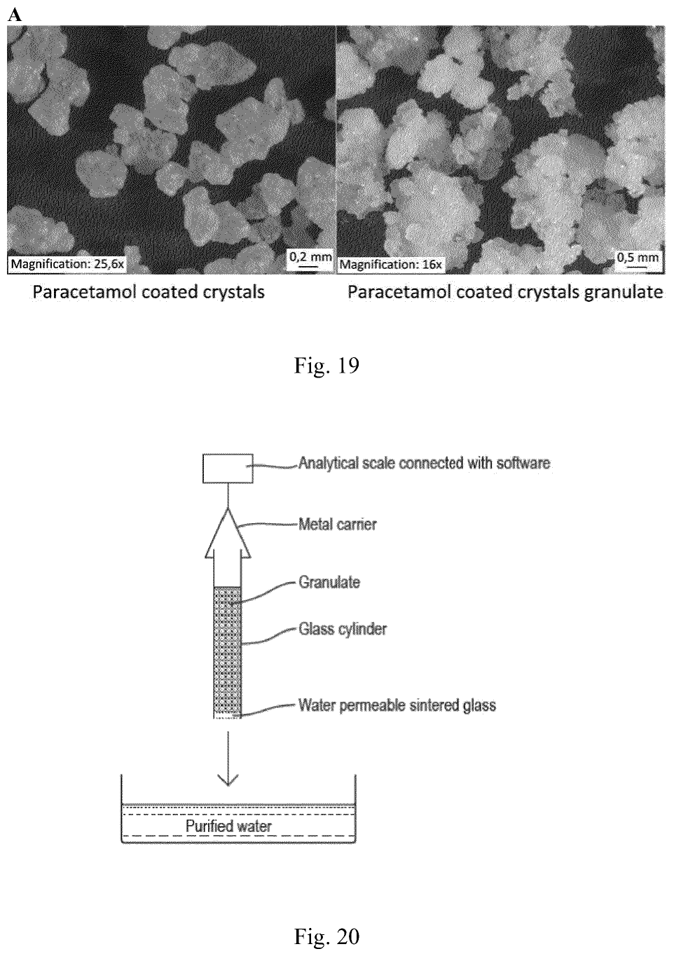

Crystals of paracetamol as API were obtained and coated with one of four different coating agents: Actimask; gelatin; PVA-PEG graft copolymer/PVA polymer (Kollicoat® Protect); and vinylpyrrolidone-vinyl acetate copolymer (Kollidon VA64). An example of the coated particles are shown in FIG. 19 (left-hand panel). Subsequently, the coated crystals were granulated in the presence of sucrose to form granules comprising 20 wt % coated and granulated paracetamol. An example of these granules is shown in FIG. 19 (right-hand panel).

Sipping pressures and flush volumes were measured as in Comparative Example 1 above. The particle size distribution, tested mass, p₁, p₂ and p₃ values from the pressure experiment, and the flush volumes, are set out in Table 9 below for both granulated particles and non-granulated (control) particles.

It is observed from Table 9 that the larger granulates have a significantly reduced maximum sipping pressure (p₂) value than the corresponding smaller non-granulated particles, require a lower flush volume, and are significantly less likely to form clogs.

Water sorption of the granulated and non-granulated particles was also tested by measurement of mass change in a particle caused by the absorption of water over time. To carry out this experiment, the test particles are placed in a glass cylinder with a water-permeable glass filter at the bottom. The cylinder is slowly lowered with controlled speed into the beaker with purified water that has been previously equilibrated to room temperature. Weight changes due to water uptake are measured when the cylinder touches the water surface. After an initial increase in particle mass, in some cases the mass then begins to decrease, which is the consequence of the particles dissolving in the water. A suitable experimental set-up is shown in FIG. 20.

TABLE 9
Experimental parameters for each of the tested granulated and non-granulated
compositions containing paracetamol as the API.

| | | | | | | | | |
|---|---|---|---|---|---|---|---|---|
| | Particle size | Mass | | | | | Reduction | Flush |
| Particles | distribution | | Clog | p₁ | p₂ | p₃ | in p₂ | volume |
| name | [μm] | [g] | formation | [mbar] | [mbar] | [mbar] | [%] | [mL] |

NON-GRANULATED COATED PARTICLES

| | | | | | | | | |
|---|---|---|---|---|---|---|---|---|
| Actimask | 250−710 | 2.79 | — — | −185 | −315 | −315 | N/A | NT |
| Gelatin | 250−710 | 2.66 | — — | −142 | −220 | −220 | N/A | >60 |
| Kollicoat® | 250−710 | 2.57 | — — | −143 | −248 | −248 | N/A | >40 |
| Protect | | | | | | | | |
| Kollidon | 250−710 | 2.60 | — — | −130 | −230 | −230 | N/A | >60 |
| VA64 | | | | | | | | |

GRANULATED PARTICLES

| | | | | | | | | |
|---|---|---|---|---|---|---|---|---|
| 20% | 500−2000 | 2.05 | — — | −81 | −200 | −134 | 33 | 20 |
| Actimask | | | | | | | | |
| 20% | 500−2000 | 1.89 | + | −60 | −184 | −97 | 16 | 50 |
| Gelatin | | | | | | | | |
| Kollicoat ® | 500−2000 | 1.83 | ++ | −53 | −150 | −95 | 40 | 30 |
| Protect | | | | | | | | |
| Kollidon | 500−2000 | 1.92 | ++ | −75 | −162 | −105 | 30 | 50 |
| VA64 | | | | | | | | |

NT = not tested. — — indicates very high levels of clogging; — indicates high levels of clogging; + indicates low levels of clogging; and ++ indicates very low levels of clogging/no clogging.

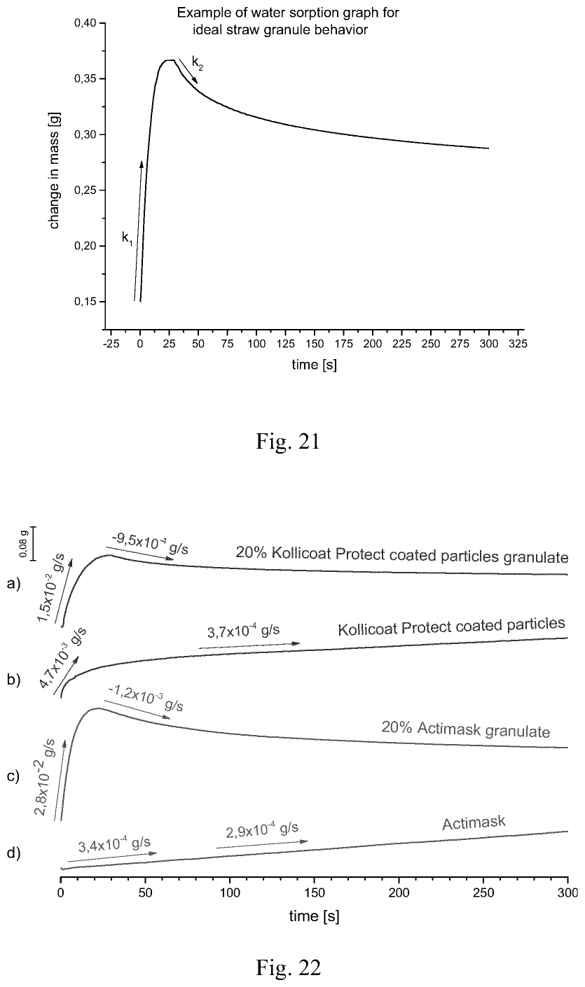

The water sorption test provides two key parameters: (a) water absorption rate (an increase of weight at the beginning of the test); and (b) rate of particle dissolution after maximum weight was achieved. The water absorption rate is expressed as mass/time ratio at the beginning of the graph which is calculated from the first several measured points using a linear regression method (slope) (parameter k₁ illustrated on FIG. 21). The mass loss rate is expressed as the value of mass/time ratio and is determined from first several measurements after the maximum mass change is achieved using linear regression (slope) (parameter k₂ illustrated on FIG. 21).

Water sorption experiments were carried out on each of the paracetamol-containing granulate and non-granulate formulations discussed above. The values of k₁ and k₂ for each sample are shown in Table 10 below.

The particle disintegration of the paracetamol-containing formulations was also measured. In this experiment, particles were dispersed in purified water as a solvent, and particle sizes were measured by a laser diffraction technique at pre-defined time intervals using a Malvern mastersizer instrument. Measurements were taken at 30 s, 1 min and 5 min after suspension formation. The results, expressed as d₅₀ values, are also shown in Table 10. D₅₀ is the particle diameter at which 50% of the sample mass is comprised of smaller particles.

It is apparent from these data that the granulated particles display (a) an improved water sorption (higher k₁ values) and (b) a more rapid dissolution/disintegration rate in water (higher k₂ values and a faster decrease in particle size in the laser diffraction test). In most cases, the absolute mean mass particle size of the granulated particles after 60 s in suspension in water is lower than the absolute mean mass particle size of the non-granulated particles, even though the granulated particles were significantly larger on average at the beginning of the experiment. This effect is unexpected and demonstrates a clear advantage to using granulated particles over non-granulated particles in a clinical setting.

TABLE 10
Results of water sorption and particle disintegration tests for each of the
tested granulated and non-granulated compositions containing paracetamol as the API.

| | | | | | |
|---|---|---|---|---|---|
| | | | Starting | | |
| | | | particle size | d₅₀ (30 s) | d₅₀ (60 s) |
| Particles name | k₁ [g/s] | k₂ [g/s] | [μm] | [μm] | [μm] |

NON-GRANULATED, COATED PARTICLES

| | | | | | |
|---|---|---|---|---|---|
| Actimask | 3.40E−04 | 2.92E−04 | 250-710 | 515 | 507 |
| Gelatin | 1.15E−03 | 3.82E−04 | 250-710 | 413 | 361 |
| Kollicoat ® | 4.74E−03 | 3.66E−04 | 250-710 | 499 | 380 |
| Protect | | | | | |
| Kollidon VA64 | 2.45E−02 | −5.89E−04 | 250-710 | 467 | 443 |

GRANULATED PARTICLES

| | | | | | |
|---|---|---|---|---|---|
| 20% Actimask | 2.78E−02 | −1.23E−03 | 500-2000 | 493 | 445 |
| 20% Gelatin | 2.36E−02 | −1.69E−03 | 500-2000 | 521 | 497 |
| Kollicoat ® | 1.46E−02 | −9.48E−04 | 500-2000 | 439 | 368 |
| Protect | | | | | |
| Kollidon VA64 | 2.12E−02 | −1.52E−03 | 500-2000 | 425 | 425 |

The water sorption curves for two of the granulated particle types (the “20% Actimask” and “20% Kollicoat® Protect” samples) and their corresponding non-granulated analogues are shown in FIG. 22. This shows clearly the improved k₁ and k₂ values for the granulated particle samples.

### Example 7: Comparison of Granulated Products with Products Available on the Market

Two commercially available products (Tachipirina 500 mg orosoluble, and Aspirin direct 500 mg) were tested for comparison with the 20% paracetamol granulates with an outer coating of sucrose described above in Example 6. Comparative results are shown in Table 11.

TABLE 11
Experimental parameters for commercially available formulations tested in the
sipping test, water sorption test and disintegration test.

| | Particle size | | | | | | Flush |
|---|---|---|---|---|---|---|---|
| | distribution | Mass | Clog | p₁ | p₂ | p₃ | volume |
| Particles name | [μm] | [g] | forming | [mbar] | [mbar] | [mbar] | [mL] |
| Tachipirina | <1400 | 2.58 | — — | −207 | −337 | −337* | — |
| 500 mg orosoluble | | | | | | | |
| Aspirin direct | <1400 | 3.16 | — — | −298 | −340 | −340* | — |
| 500 mg | | | | | | | |

| | | | Starting | | |
|---|---|---|---|---|---|
| | | | particle | d₅₀ | d₅₀ |
| | | | size | (30 s) | (60 s) |
| Particles name | k₁ [g/s] | k₂ [g/s] | [μm] | [μm] | [μm] |
| Tachipirina | 5.76E−04 | −9.27E−05 | 250-710 | 400 | 384 |
| 500 mg orosoluble | | | | | |
| Aspirin direct | 1.84E−04 | 3.51E−04 | 250-710 | 460 | 437 |
| 500 mg | | | | | |

— — indicates very high levels of clogging; — indicates high levels of clogging; + indicates low levels of clogging; and ++ indicates very low levels of clogging/no clogging. *p₃ value is equivalent to p₂ value meaning sipping test was prematurely aborted due to high sipping pressures. Straw was not emptied at all.

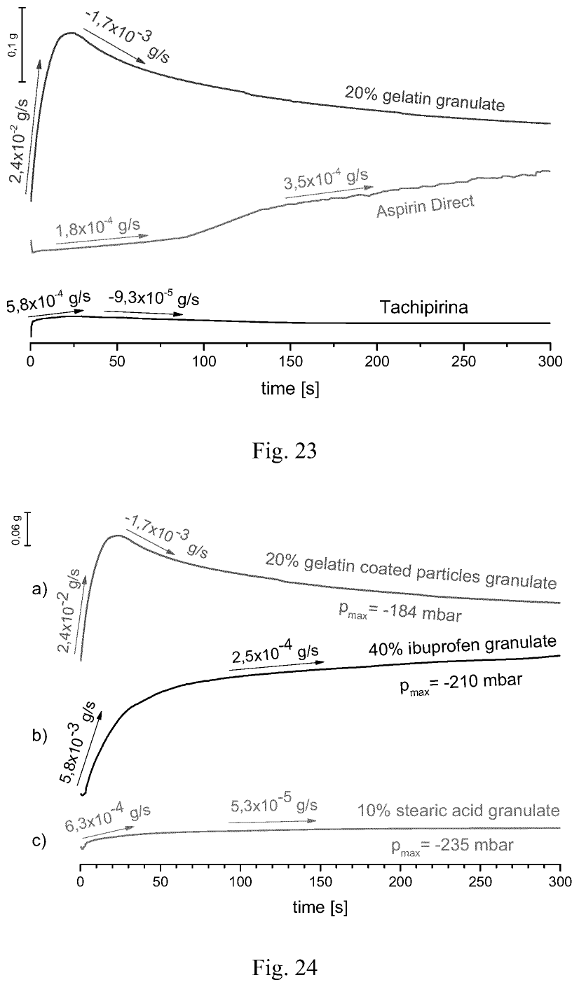

FIG. 23 additionally shows water sorption curves for Tachipirina and Aspirin Direct, compared with a gelatin granulate (20% by weight of coated core phase) of the present invention.

It is observed that the granulated formulations of the present invention provide a superior balance of properties to the commercially available formulations. In particular, the formulations of the present invention exhibit (a) a lower maximal sipping pressure, which enables the straw to be emptied in a lower flush volume, (b) significantly larger k₁ and k₂ values, providing more rapid water sorption and particle dissolution, and (c) more rapid disintegration of particles as demonstrated by the laser diffraction test.

### Example 8: Investigating the Effect of API Content on Water Sorption Properties

An experiment was also carried out to determine the optimal amount of API by weight in the granulated formulations. Three granulated formulations were prepared as follows: (a) sucrose granulate with 20% content of gelatin-coated paracetamol particles, prepared in accordance with the two-step coating and granulation procedure of the present invention; (b) sucrose granulate with 40% uncoated ibuprofen content; and (c) sucrose granulate with 10% uncoated stearic acid content.

The water sorption curves for each of these samples are shown in FIG. 24. It is observed that formulations (a) and (b) have more optimal water sorption properties than formulation (c), and that formulations (a) and (c) have more optimal disintegration rates than formulation (b).

### Example 9: Effect of Granule Particle Size and Additional Excipients on Granule Properties

Effect of particle size on properties of granules not containing an API

In this example, preferred drinking straws as shown in FIG. 1 and described in detail above were employed. Each straw was pre-filled with c. 1-2 grams of one of the various formulations set out in Table 12 below. The formulations differ from one another in the nature of the excipient, carrier, diluent, binder or disintegrant used, the nature of the surfactant added, and the particle size. The formulations were prepared by a granulation method using a PROCEPT 4M8-TriX Formatrix high shear granulator, operated under the following conditions:
High shear granulator: PROCEPT 4M8-TriX Formatrix
Temperature: 23° C. (room temperature)
Impeller speed: 800 rpm
Chopper speed: 1500 rpm
Vessel volume: 1 L
Binding dispersion flow rate: 2.1 g/min
Nozzle type: Two-fluid nozzle with 0.8 mm inner diameter
Drying type: Tray dryer
Drying duration and temperature: 1 hr 15 mins at 50.0° C.

After granulation, the formulations were subjected to a dry sieving process using mechanical agitation, which is a well-known process to a person skilled in the art (see, e.g., http://www.pharmacopeia.cn/v29240/usp29nf24s0_c786.html), in order to obtain the desired particle size groups.

Each straw was then used as follows. The lower end of the straw was placed into a glass containing approximately 100 mL of water. The upper end of the straw was placed into the oral cavity of a human subject, and the subject drank water from the glass by sucking on the upper end of the straw. Drinking continued until all of the formulation in the straw had been fully consumed orally by the subject (determined by the time to complete dissolution of the formulation).

Table 12 below reports the following observations for each of the tested formulations:
1. Whether clogging of the straw (due to cake formation of the formulation) was observed, under visual inspection. −− indicates very high levels of clogging; − indicates high levels of clogging; + indicates low levels of clogging; and ++ indicates very low levels of clogging/no clogging.
2. Whether the flow of liquid through the straw was impeded due to resistance imparted by the formulation, based on how hard the subject had to suck on the upper end of the straw to drink the water. Flow was graded on a four-point scale: “excellent”; “good”; “average”; “poor” and “very poor”.
3. The emptying volume of the straw, i.e. the amount of water (in mL) that the subject had to drink in order to ingest the entire formulation initially present in the straw.

TABLE 12
Behaviour of different formulations when used in a drinking straw. Letters a-e for
each numbered example (Ex.) denote the five different particle size distributions
(a = 1400-1120; b = 1120-1000; c = 1000-710; d = 710-500; e = 500-250).
The “particle size” indicates that at least 95% of the particles have a size
falling within the stated range. SDS = sodium dodecyl sulfate;
PVP K30 = poly(vinyl pyrrolidone) K30; conc. = concentration.

| | Parameters of Formulation | | | | Behaviour/Observations | | |
|---|---|---|---|---|---|---|---|
| | | | | Binder, | | | |
| | | | | surfactant | | | |
| | | | | solution | | | |
| | | | | conc. | | | |
| | | | Particle | (added | | | Emptying |
| | | | size | solution | | | volume |
| Ex. | Composition | | (μm) | mass) | Clogging | Flow | (mL) |
| 1a-e | 200 | g isomalt | 1400-1120 | 5% SDS | ++ | Good | <50 |
| | 1.03 | g SDS | 1120-1000 | 7% PVP K30 | ++ | Excellent | ≥50 |
| | 1.44 | g PVP K30 | 1000-710 | (20.6 g) | + | Average | <50 |
| | | | 710-500 | | — | Poor | <50 |
| | | | 500-250 | | — — | Poor | ≥50 |
| 2a-e | 200 | g isomalt | 1400-1120 | 7% PVP K30 | + | Poor | * |
| | 1.34 | g PVP K30 | 1120-1000 | (19.2 g) | + | Good | >50 |
| | | | 1000-710 | | — | Poor | * |
| | | | 710-500 | | ++ | Good | ≥50 |
| | | | 500-250 | | — — | Very poor | <50 |
| 3a-e | 170 | g isomalt | 1400-1120 | 5% SDS | ++ | Excellent | >50 |
| | 30 | g citric acid | 1120-1000 | 7% PVP K30 | ++ | Excellent | >50 |
| | 0.43 | g SDS | 1000-710 | (8.6 g) | ++ | Average | >50 |
| | 0.60 | g PVP K30 | 710-500 | | + | Average | <50 |
| | | | 500-250 | | — — | Very poor | <50 |
| 4a-e | 170 | g isomalt | 1400-1120 | 7% PVP K30 | ++ | Good | ≥50 |
| | 30 | g citric acid | 1120-1000 | (8.3 g) | + | Good | >50 |
| | 0.58 | g PVP K30 | 1000-710 | | + | Good | ≥50 |
| | | | 710-500 | | + | Average | <50 |
| | | | 500-250 | | — — | Very poor | >50 |
| 5a-e | 190 | g isomalt | 1400-1120 | 5% SDS | \hat{ } | \hat{ } | \hat{ } |
| | 10 | g citric acid | 1120-1000 | 7% PVP K30 | \hat{ } | \hat{ } | \hat{ } |
| | 0.75 | g SDS | 1000-710 | (15.0 g) | ++ | Excellent | >50 |
| | 1.05 | g PVP K30 | 710-500 | | + | Average | ≥50 |
| | | | 500-250 | | — | Poor | >50 |
| 6a-e | 190 | g isomalt | 1400-1120 | 7% PVP K30 | \hat{ } | \hat{ } | \hat{ } |
| | 10 | g citric acid | 1120-1000 | (14.3 g) | \hat{ } | \hat{ } | \hat{ } |
| | 1.00 | g PVP K30 | 1000-710 | | + | Average | ≥50 |
| | | | 710-500 | | ++ | Good | ≥50 |
| | | | 500-250 | | — — | Very poor | >50 |
| 7a-e | 200 | g sucrose | 1400-1120 | 5% SDS | ++ | Excellent | ≥50 |
| | 0.76 | g SDS | 1120-1000 | 7% PVP K30 | ++ | Excellent | ≥50 |
| | 1.06 | g PVP K30 | 1000-710 | (15.2 g) | ++ | Excellent | ≥50 |
| | | | 710-500 | | ++ | Good | <50 |
| | | | 500-250 | | — — | Poor | <50 |
| 8a-e | 200 | g sucrose | 1400-1120 | 7% PVP K30 | + | Poor | <50 |
| | 1.25 | g PVP K30 | 1120-1000 | (17.8 g) | + | Average | >50 |
| | | | 1000-710 | | + | Good | <50 |
| | | | 710-500 | | + | Average | <50 |
| | | | 500-250 | | — — | Poor | <50 |
| 9a-e | 170 | g sucrose | 1400-1120 | 5% SDS | ++ | Excellent | ≥50 |
| | 30 | g citric acid | 1120-1000 | 7% PVP K30 | + | Good | <50 |
| | 0.53 | g SDS | 1000-710 | (10.6 g) | ++ | Good | ≥50 |
| | 0.74 | g PVP K30 | 710-500 | | ++ | Excellent | ≥50 |
| | | | 500-250 | | — — | Poor | <50 |
| 10a-e | 170 | g sucrose | 1400-1120 | 7% PVP K30 | ++ | Good | ≥50 |
| | 30 | g citric acid | 1120-1000 | (7.7 g) | + | Average | ≥50 |
| | 0.54 | g PVP K30 | 1000-710 | | — | Poor | ≥50 |
| | | | 710-500 | | + | Average | ≥50 |
| | | | 500-250 | | — — | Very poor | ≥50 |
| 11a-e | 190 | g sucrose | 1400-1120 | 5% SDS | ++ | Excellent | ≥50 |
| | 10 | g citric acid | 1120-1000 | 7% PVP K30 | ++ | Excellent | ≥50 |
| | 0.60 | g SDS | 1000-710 | (12.0 g) | + | Good | ≥50 |
| | 0.84 | g PVP K30 | 710-500 | | — | Poor | ≥50 |
| | | | 500-250 | | — — | Very poor | ≥50 |
| 12a-e | 190 | g sucrose | 1400-1120 | 7% PVP K30 | ++ | Excellent | <50 |
| | 10 | g citric acid | 1120-1000 | (11.3 g) | ++ | Excellent | ≥50 |
| | 0.79 | g PVP K30 | 1000-710 | | + | Average | ≥50 |
| | | | 710-500 | | + | Good | ≥50 |
| | | | 500-250 | | — — | Very poor | ≥50 |

* denotes that the particles attached to the walls of the straw, and that it was therefore not possible to empty the straw. \hat{ } indicates that there was not enough of this particle fraction to obtain a meaningful test result.

In general, it can be observed that formulations having a particle size of <500 μm tend to exhibit high levels of clogging, and poor or very poor flow of liquid through the straw when in use.

### Effect of API and Excipients on Properties of Granules

Each straw (as above) was pre-filled with c. 1.5-2 grams of one of the various formulations set out in Table 13 below. The formulations differ from one another in the presence/absence, nature and amount of the API and surfactant added, and the particle size. Cefprozil monohydrate was used as an example API. The formulations were prepared via a typical granulation and sieving process as described above for the formulations without API.

TABLE 13
Composition of the exemplar formulations. SDS = sodium dodecyl sulfate;
PVP K30 = poly(vinyl pyrrolidone) K30; PVP F90 = poly(vinyl pyrrolidone) F90. Each
of the samples was produced having two different particle size distributions, 250-
500 pm and 1120-1400 μm.

| | | % by weight of |
|---|---|---|
| Sample | Components | each component |
| VZ48 | Sucrose | 100 |
| VZ49 | Sucrose/ SDS | 99.6/0.4 |
| VZ30 | Sucrose/PVP K30 | 99.4/0.6 |
| VZ40 | Sucrose/ PVP F90 | 99.6/0.4 |
| VZ29 | Sucrose/PVP K30/ SDS | 99.1/0.5/0.4 |
| VZ41 | Sucrose/ PVP F90/ SDS | 99.3/0.4/0.3 |
| VZ51 | Sucrose/ cefprozil monohydrate | 70/30 |
| VZ52 | Sucrose/ cefprozil monohydrate/ SDS | 69.6/30/0.4 |
| VZ47 | Sucrose/ cefprozil monohydrate/PVP K30/ SDS | 69.1/30/0.5/0.4 |
| VZ35 | Sucrose/ citric acid/ PVP K30/ SDS | 94.3/5/0.4/0.3 |
| VZ31 | Sucrose/ citric acid/ PVP K30/ SDS | 84.3/15/0.4/0.3 |
| VZ33 | Sucrose/ citric acid/ PVP K30 | 94.6/5/0.4 |
| VZ32 | Sucrose/ citric acid/ PVP K30 | 84.7/15/0.3 |

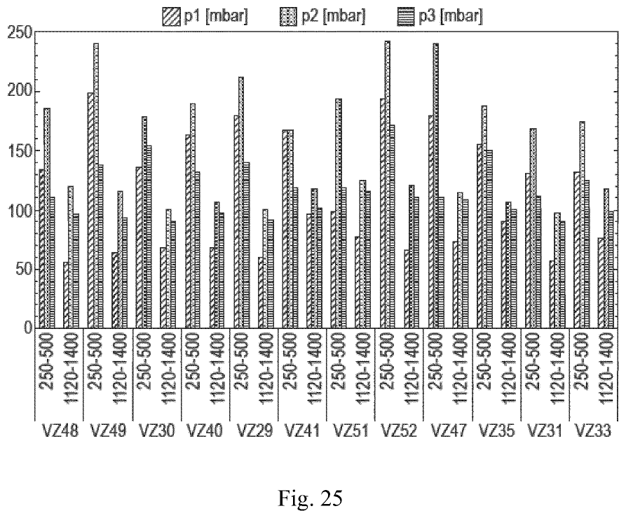

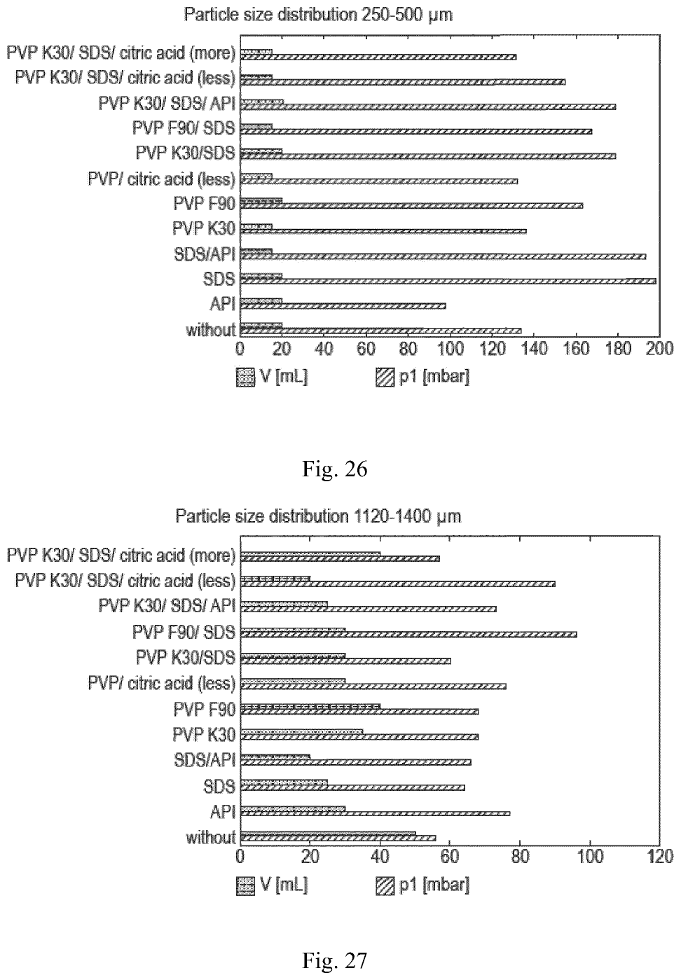

Sipping pressures and flush volumes were measured as in Comparative Example 1 above. The particle size distribution, tested mass, p₁, p₂ and p₃ values from the pressure experiment, and the flush volumes, are set out in Tables 14 and 15 below. The data in Table 3 is illustrated in graphical format in FIG. 25, and the data in Table 4 is illustrated in graphical format in FIG. 26 (for the formulations having a particle size of 250-500 μm) and FIG. 27 (for the formulations having a particle size of 1120-1400 μm).

From these data, the following conclusions may be drawn in respect of the formulations having a smaller particle size (250-500 μm):
The addition of API has a postive effect on the vacuum required to drink from the straw, with the pressure p1 decreasing by c. 30 mbar, without an impact on flush volume (V).
Addition of SDS (which acts as a surfactant) increases the absolute pressure p1 (i.e. resistivity to drinking through the straw) but does not affect the flush volume.
Addition of both API and SDS increases the pressure p1 as for SDS alone, but the flush volume is reduced.
Use of PVP K30 as a binder does not increase the pressure p1, but use of PVP F90 as a binder does. The pressure p1 increases when either PVP K30 or PVP F90 is added in combination with SDS.
If citric acid is added in a small amount there is little effect on p1 and a small decrease in flush volume. If citric acid is added in a larger amount, it can mitigate against the increased p1 caused by SDS.
P₁ does not increase when API is added in combination with PVP K30 and SDS.

Cake formation was observed in all cases where the particle size was small (250-500 μm), but not in any case where the particle size was large (1120-1400 μm). The pressure p1 is notably lower when larger particles were used as opposed to the corresponding formulations comprising smaller particles. Conversely, the flush volume increases with particle size.

TABLE 14
Experimental parameters for each of the tested compositions described in
Table 13 above. NT = not tested.

| | | Particle | Mass in | | | | | Cake/Clog |
|---|---|---|---|---|---|---|---|---|
| | | size | straw | p₁ | p₂ | p₃ | V | formation |
| Sample | Composition | (μm) | (g) | (mbar) | (mbar) | (mbar) | (mL) | observed? |
| VZ48 | Sucrose | 250-500 | 2.273 | 134 | 185 | 110 | 20 | yes |
| | | 1120-1400 | 2.344 | 56 | 120 | 96 | 50 | no |
| VZ49 | Sucrose, SDS | 250-500 | 2.422 | 198 | 240 | 138 | 20 | yes |
| | | 1120-1400 | 2.284 | 64 | 115 | 93 | 25 | no |
| VZ30 | Sucrose, PVP K30 | 250-500 | 2.164 | 136 | 178 | 154 | 15 | yes |
| | | 1120-1400 | 1.887 | 68 | 100 | 90 | 35 | no |
| VZ40 | Sucrose, PVP F90 | 250-500 | 2.103 | 163 | 189 | 131 | 20 | yes |
| | | 1120-1400 | 1.555 | 68 | 106 | 97 | 40 | no |
| VZ29 | Sucrose, PVP K30, SDS | 250-500 | 2.063 | 179 | 212 | 140 | 20 | yes |
| | | 1120-1400 | 1.836 | 60 | 100 | 91 | 30 | no |
| VZ41 | Sucrose, PVP F90, SDS | 250-500 | 2.008 | 167 | 167 | 119 | 15 | yes |
| | | 1120-1400 | 1.563 | 96 | 118 | 101 | 30 | no |
| VZ51 | Sucrose, cefprozil | 250-500 | 1.916 | 98 | 193 | 119 | 20 | yes |
| | monohydrate | 1120-1400 | 1.543 | 77 | 125 | 115 | 30 | no |
| VZ52 | Sucrose, cefprozil | 250-500 | 2.029 | 193 | 242 | 171 | 15 | yes |
| | monohydrate, SDS | 1120-1400 | 1.030 | 66 | 120 | 110 | 20 | no |
| VZ47 | Sucrose, cefprozil SDS | 250-500 | 2.098 | 179 | 240 | 110 | 20 | yes |
| | monohydrate, PVP K30, | 1120-1400 | 1.682 | 73 | 114 | 108 | 25 | no |
| VZ35 | Sucrose, citric acid, PVP | 250-500 | 1.750 | 155 | 187 | 150 | 15 | yes |
| | K30, SDS | 1120-1400 | 1.681 | 90 | 106 | 100 | 20 | no |
| VZ31 | Sucrose, citric acid, PVP | 250-500 | 1.897 | 131 | 168 | 111 | 15 | yes |
| | K30, SDS | 1120-1400 | 1.795 | 57 | 97 | 90 | 40 | no |
| VZ33 | Sucrose, citric acid, PVP | 250-500 | 1.804 | 132 | 174 | 125 | 15 | yes |
| | K30 | 1120-1400 | 1.623 | 76 | 118 | 98 | 30 | no |
| VZ32 | Sucrose, citric acid, PVP | 250-500 | 2.532 | 130 | 211 | 148 | 15 | yes |
| | K30 | 1120-1400 | NT | NT | NT | NT | NT | NT |

TABLE 15
Values of V and p₁ for each of the different
exemplar formulations described in Table 13 above.

| | | Particle | | | Particle | | |
|---|---|---|---|---|---|---|---|
| Composition of | | size | p₁ | V | size | p₁ | V |
| formulation | Sample | (μm) | (mbar) | (mL) | (μm) | (mbar) | (mL) |
| Excipient only | VZ48 | 250-500 | 134 | 20 | 1120-1400 | 56 | 50 |
| +API | VZ51 | 250-500 | 98 | 20 | 1120-1400 | 77 | 30 |
| +SDS | VZ49 | 250-500 | 198 | 20 | 1120-1400 | 64 | 25 |
| +SDS and API | VZ52 | 250-500 | 193 | 15 | 1120-1400 | 66 | 20 |
| +PVP K30 | VZ30 | 250-500 | 136 | 15 | 1120-1400 | 68 | 35 |
| +PVP F90 | VZ40 | 250-500 | 163 | 20 | 1120-1400 | 68 | 40 |
| +PVP K30 and | VZ33 | 250-500 | 132 | 15 | 1120-1400 | 76 | 30 |
| small amount of | | | | | | | |
| citric acid (5%) | | | | | | | |
| +PVP K30 | VZ29 | 250-500 | 179 | 20 | 1120-1400 | 60 | 30 |
| and SDS | | | | | | | |
| +PVP F90 and SDS | VZ41 | 250-500 | 167 | 15 | 1120-1400 | 96 | 30 |
| +PVP K30, SDS | VZ47 | 250-500 | 179 | 20 | 1120-1400 | 73 | 25 |
| and API | | | | | | | |
| +PVP K30, SDS | VZ35 | 250-500 | 155 | 15 | 1120-1400 | 90 | 20 |
| and small amount | | | | | | | |
| of citric acid (5%) | | | | | | | |
| +PVP K30, SDS | VZ31 | 250-500 | 131 | 15 | 1120-1400 | 57 | 40 |
| and greater amount | | | | | | | |
| of citric acid (15%) | | | | | | | |

The following conclusions may be drawn in respect of the formulations having a larger particle size (1120-1400 μm):
The addition of API in general increases the pressure p1, but reduces the volume of water required to fully administer the formulation to a subject.
The addition of SDS (which acts as a surfactant) slightly increases the required drinking pressure but not as much as the addition of API.
The addition of SDS results in a large reduction inbthe flush volume.
The addition of SDS in combination with the API mitigates against the increased p1 resulting from API addition, and the flush volume is further reduced.
The addition of PVP as a binder generally increases the vacuum necessary for drinking, and the addition of PVP F90 also leads to a slight increase in the flush volume.
If citric acid is added in a large quantity, this can mitigate against the increased p₁ caused by SDS; however, the flush volume is increased. If citric acid is added in a smaller amount in combination with SDS and PVP, this results in an increase in the vacuum required to drink the water.

In a further experiment, an additional five granule samples were investigated in five different particle size distributions (250-500 μm, 500-710 μm, 710-1000 μm, 1000-1120 μm, and 1120-1400 μm). These samples were as follows:

(1) Placebo sucrose granulate;

(2) Placebo sucrose granulate with SDS;

(3) Sucrose granulate with cefprozil monohydrate (30 wt %);

(4) Sucrose graunlate with SDS and cefprozil monohydrate (30 wt %); and

(5) Sucrose granulate with SDS, cefprozil monohydrate (30 wt %) and PVP K30.

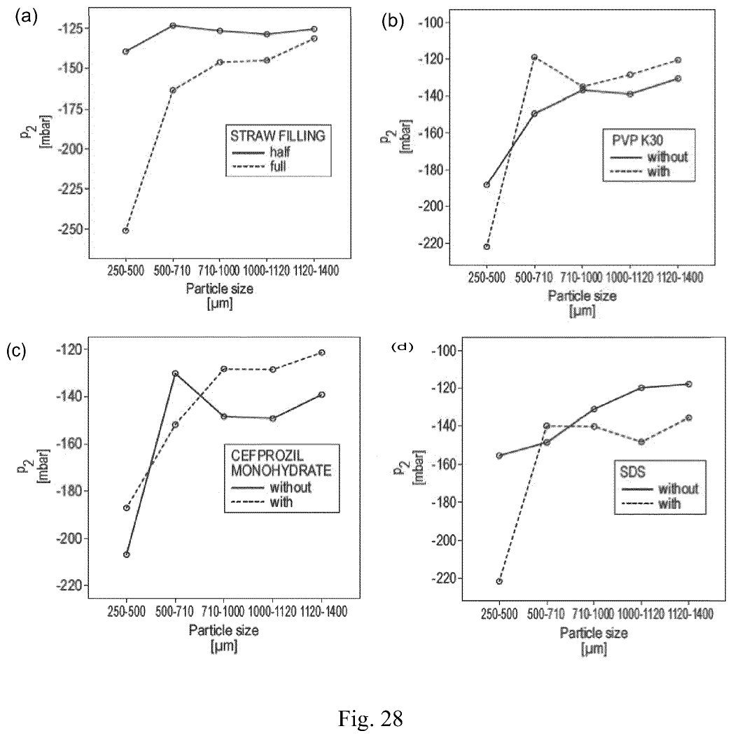

The effect of (a) the amount of placebo granulates filled in the straw (full or half), (b) the addition of PVP K30 (formulation (4) vs (5)), (c) the addition of cefprozil monohydrate (formulation (1) vs (3)), and (d) the addition of SDS (formulation (1) vs (2)) on the sipping pressure p₂ and the flush volume V is shown in FIGS. 28(a)-(d) and 29(a)-(d) respectively.

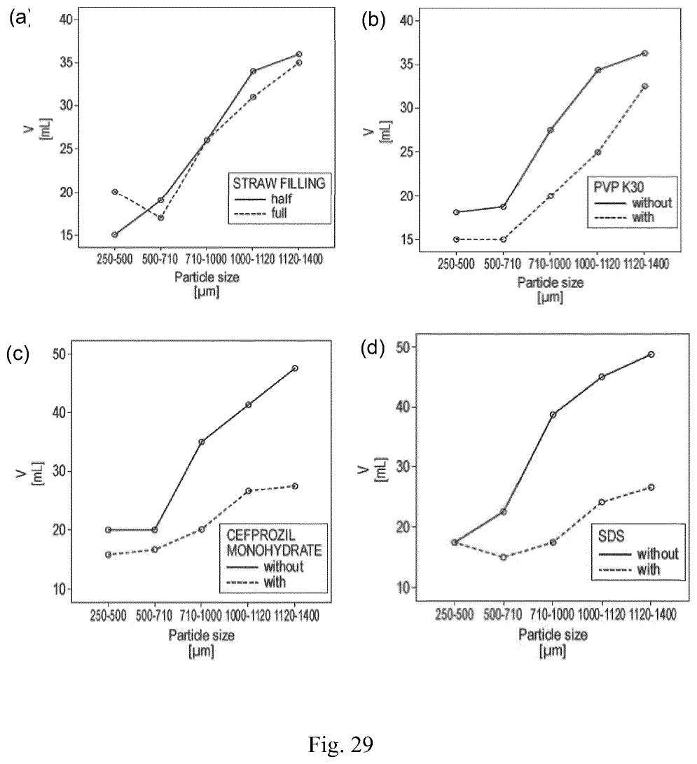

As can be seen from FIG. 28(a), the maximum sipping pressure correlates with the straw filling (i.e. the proportion of the straw that is filled with the particle formulation). It is observed that p₂ is higher when a straw is half-full compared to completely full. Sipping pressures for half straw filling almost do not differ between different particle size distributions. Smaller particle sizes in a full filled straw, on the other hand, increase the sipping pleasure greatly. For flush volume values, there were no significant differences found due to straw filling. Smaller particles may be uptaken orally using a smaller volume of fluid than the larger particle distributions. In line with the above results, it was also observed that addition of SDS as surfactant greatly reduces flush volume values (see FIG. 29(d)) but slightly increases the sipping pressure. Without wishing to be bound by any particular theory, it is thought that this is due to the creation of less dense and more porous granules in the presence of SDS.

It was also found that cefprozil (as an example of a water-soluble API) significantly reduces flush volumes compare to placebo granulates. In the case of cefprozil addition, a lower sipping pressure was also obtained for granule particle sizes greater than 710 μm.

1. A pharmaceutical formulation suitable for use in a straw suitable for oral administration of said pharmaceutical formulation, wherein the pharmaceutical formulation is solid and comprises granules, and the granules comprise:
(a) a core comprising an active pharmaceutical ingredient (API), food supplement or vitamin;
(b) optionally, a first coating layer surrounding the core; and
(c) a second coating layer surrounding the first coating layer and/or the core, the second coating layer comprising particles of a sugar, a sugar alcohol, or any mixture thereof.

2. The pharmaceutical formulation according to claim 1, wherein the second coating layer is obtainable using a wet coating method in a high shear mixer or a liquid-assisted coating method in a high-shear mixer, preferably wherein the second coating layer is obtainable using a wet coating method in a high shear mixer, preferably wherein the wet coating method comprises spraying or dropping of a liquid binding solution onto a core optionally coated with a first coating layer.

3. (canceled)

4. The pharmaceutical formulation according to claim 1, wherein a first coating layer is present and the first coating layer comprises a polymer, optionally wherein the polymer is selected from gelatins, ovalbumin, soybean proteins, gum arabic, non-sucrose fatty acid esters, starches, modified starches, cellulose, methylcellulose (MC), ethylcellulose (EC), hydroxyethylcellulose (HEC), hydroxypropylcellulose (HPC), hydroxypropylmethylcellulose (HPMC), polyvinyl alcohol, polycarbophil, polyethylene glycol (PEG), polyethylene oxides, polyoxyalkylene derivatives, polymethacrylates, poly(vinyl pyrrolidone) (PVP), polyvinyl alcohol (PVA), polyvinyl acetate (PVAc), PVP-vinylacetate-copolymer (PVP-VA), a vinylpyrrolidone-vinyl acetate copolymer (such as Kollidon® VA 64), sodium carboxymethyl cellulose, sodium alginate, xantham gum, locust bean gum, chitosan, cross-linked high amylase starch, cross-linked polyacrylic acid (carbopol), a polyvinylalcohol-polyethyleneglycol-copolymer and mixtures thereof.

5. The pharmaceutical formulation according to claim 1, wherein a further coating layer is present between the first coating layer and the second coating layer, optionally wherein the further coating layer comprises a hydrophilic polymer that is partially soluble in water, preferably wherein the further coating layer comprises poly(vinyl alcohol), ammonio methacrylate Type A, ammonio methacrylate Type B, or a mixture thereof.

6. (canceled)

7. The pharmaceutical formulation according to claim 1, wherein a first coating layer is present and the first coating layer is obtainable by fluid bed coating or high shear melt coating.

8. The pharmaceutical formulation according to claim 1, wherein the core is substantially spherical.

9. The pharmaceutical formulation according to claim 1, wherein the first coating layer is an enteric coating, a physical barrier coating and/or a taste masking agent.

10. The pharmaceutical formulation according to claim 1, wherein the first coating layer comprises two or more discrete sublayers.

11. The pharmaceutical formulation according to claim 1, wherein at least 90% of the granules by number have a diameter of greater than 200 μm and in which at least 90% of the granules by number have a diameter of less than 1400 μm.

12. The pharmaceutical formulation according to claim 1, wherein the particles of a sugar, a sugar alcohol, or any mixture thereof are from 5 to 1000 times smaller in diameter than the core, and/or wherein the diameter of the granule is up to five times greater than the diameter of the core.

13. (canceled)

14. The pharmaceutical formulation according to claim 1, wherein the sugar, sugar alcohol or any mixture thereof is selected from monosaccharides, disaccharides, polysaccharides, glucose, arabinose, lactose, dextrose, sucrose, fructose, maltose, trehalose, dextrins, galactose, mannitol, erythritol, maltitol, isomaltitol, sorbitol, xylitol, lactitol, and mixtures thereof, and preferably wherein the second coating layer comprises maltitol, mannitol, trehalose, or a mixture thereof, preferably wherein the second coating layer comprises (i) maltitol, trehalose, or a mixture thereof and (ii) erythritol.

15. (canceled)

16. The pharmaceutical formulation according to claim 1, wherein the first coating layer is a smooth layer when observed using optical or electron microscopy, and/or wherein the second coating layer is a rough layer when observed using optical or electron microscopy.

17. (canceled)

18. A device suitable for oral administration of a pharmaceutical formulation, wherein the device contains the pharmaceutical formulation of claim 1.

19. A device according to claim 18, wherein the device is a straw optionally wherein the straw comprises:
(a) a first straw segment, which contains the pharmaceutical formulation and has an integrally formed cross-slit valve at one end; and
(b) a second straw segment, which has an integrally formed cross-slit valve at one end;
wherein the ends of the first and second straw segments that do not have integrally formed cross-slit valves are coupled to one another.

20. (canceled)

21. A straw according to claim 19, wherein:
(i) at least one of the straw segments is tapered such that the cross-sectional area of the opening within the straw is smaller at the end having the cross-slit valve than at the end that is coupled to the other straw segment, optionally wherein at least one of the straw segments has a frusto-conical shape; and/or
(ii) the first straw segment is directly coupled to the second straw segment; and/or
(iii) the first straw segment is coupled to the second straw segment by at least one of a snap-fit connection, a press-fit connection, a friction fit connection, a weld and an adhesive; and/or
(iv) the first and second straw segments each have an integrally formed element at the ends that are coupled to one another, the elements configured to enable the first and second straw segments to be coupled to one another; and/or
(v) the cross-slit valves are co-moulded to the ends of the first and second straw segments; and/or
(vi) the cross-slit valves are formed in a membrane formed from a thermoplastic elastomer material, optionally wherein the membrane of the cross-slit valve formed on one of the first and second straw segments has a convex shape, further optionally wherein the membrane of the cross-slit valve formed on the other of the first and second straw segments has a concave shape; and/or
(vii) at least one of the first and second straw segments comprises a straw body to which the cross-slit valve is attached by molecular adhesion, optionally wherein the straw body is formed from a thermoplastic material and the cross-slit valve is formed from a different material, further optionally wherein the surface of the end of the straw body to which the cross-slit valve is attached has at least one recess configured to increase the area of contact between the straw body and the cross-slit valve.

22. A straw according to claim 19, wherein the pharmaceutical formulation comprises from 0.001% to 90% by weight and preferably from 5% to 60% by weight of the API, food supplement or vitamin.

23. A straw according to claim 19, wherein the straw is configured such that oral administration of at least 90% of the formulation is achieved during use when a volume of 50 mL or less of aqueous solvent, preferably 30 mL or less, and most preferably 20 mL or less, is passed through the straw.

24. A straw according to claim 19, wherein the straw is configured such that when aqueous solvent is passed through the straw for 60 seconds, the d₅₀ value of the resulting formulation is 100 μm or more, and preferably from 200 μm to 300 μm.

25. (canceled)

26. A method of treating a condition in a subject in need thereof, said method comprising oral administration of a pharmaceutical formulation using a straw as defined in claim 19, wherein the pharmaceutical formulation comprises an API, and the straw contains the pharmaceutical formulation.

27. (canceled)

28. A method of preparing a pharmaceutical formulation according to claim 1, said method comprising:
(a) providing granules, crystals or pellets of an active pharmaceutical ingredient (API), food supplement or vitamin;
(b) optionally, applying a first coating layer to the said granules, crystals or pellets, optionally using fluid bed coating or high shear melt coating; and
(c) applying a second coating layer using a wet coating method in a high shear mixer, wherein the second coating layer comprises particles of a sugar, sugar alcohol, or any mixture thereof.
